(19) United States
(12) Patent Application Publication
(10) Pub. No.: US 2025/0325696 A1
(43) Pub. Date: Oct. 23, 2025

(54) MUTANT OF ADENO-ASSOCIATED VIRUS AND USE THEREOF

(71) Applicant: PACKGENE BIOTECH CO., LTD., Guangzhou, Guangdong (CN)

(72) Inventors: Huapeng Li, Guangzhou, Guangdong (CN); Ye Bu, Guangzhou, Guangdong (CN); Yujian Zhong, Guangzhou, Guangdong (CN); Zhiyong Dai, Guangzhou, Guangdong (CN); Youwei Zhang, Guangzhou, Guangdong (CN); Junlin Chen, Guangzhou, Guangdong (CN); Yue Pan, Guangzhou, Guangdong (CN); Huan Chen, Guangzhou, Guangdong (CN)

(21) Appl. No.: 19/254,091

(22) Filed: Jun. 30, 2025

Related U.S. Application Data
(63) Continuation of application No. PCT/CN2023/072583, filed on Jan. 17, 2023.

(30) Foreign Application Priority Data
Dec. 30, 2022 (CN) 202211731180.1

(51) Int. Cl. A61K 48/00; C07K 14/005; C12N 15/86

(52) U.S. Cl. CPC A61K 48/0041; C07K 14/005; C12N 15/86; C12N 2750/14122; C12N 2750/14143; C12N 2750/14145; C12N 2750/14151

(57) ABSTRACT

The present application provides virions and uses thereof in the biological field. The present application specifically provides multiple mutants of an adeno-associated virus with high muscle targeting, quite low liver tropism, and better specificity, and applications thereof. Recombinant adeno-associated viral vectors (rAAV) are constructed using the mutants of the AAV capsid protein provided in this application. The rAAV is not only effective in mouse muscles, but also exhibits better muscle targeting and low liver toxicity in non-human primates (NHP), providing better safety and a wide range of applications.

### CROSS-REFERENCE TO RELATED APPLICATIONS

The present application is a Continuation Application of PCT application No. PCT/CN2023/072583 filed on Jan. 17, 2023, which claims the benefit of Chinese Patent Application No. 202211731180.1 filed on Dec. 30, 2022. The contents of the above-identified applications are hereby incorporated by reference.

### REFERENCE TO SEQUENCE LISTING

This application includes a Sequence Listing filed electronically as an XML file named “U.S. Pat. No. 2,401,292H-PCT_SL.xml”, created on May 22, 2025, with a size of 67,250 bytes. The Sequence Listing is incorporated herein by reference.

### TECHNICAL FIELD

The present application relates to virions and applications thereof in the field of biology, and in particular to a mutant of an adeno-associated virus (AAV) having low liver tropism and high targeting to muscle. The mutant has great application prospects in mice, non-human primates (NHPs), and even in human clinical practice.

### BACKGROUND

Adeno-associated virus (AAV) is a small non-enveloped virus encapsulating a linear single-stranded DNA genome. It belongs to the genus Dependovirus of the family Parvoviridae and requires a helper virus (usually an adenovirus) to participate in replication. The AAV genome is a single-stranded DNA fragment contained in a capsid of a non-enveloped virus and can be divided into three functional regions: two open reading frames (Rep gene, Cap gene) and the inverted terminal repeat (ITR) sequence. Recombinant adeno-associated viral (rAAV) vectors are derived from a wild-type adeno-associated virus which is non-pathogenic. Because rAAV has the advantages of a wide host range, non-pathogenicity, low immunogenicity, long-term stable expression of exogenous genes, good diffusion performance, and stable physical properties, it is widely used as a gene transfer vector in gene therapy and vaccine research. In medical research, rAAV is used in gene therapy research for a variety of diseases (including in vivo and in vitro experiments), such as gene function research, disease model construction, and preparation of gene knockout mice.

One of the severe problems faced by systemic delivery of high-dose drugs is drug toxicity, particularly the problem of hepatotoxicity (liver toxicity), which is especially significant. For example, Astellas' gene therapy drug AT132 was used to treat X-linked myotubular myopathy. The drug employed an AAV8 vector to deliver the myotubularin gene to the skeletal muscle, thereby increasing the expression of myotubularin in the tissue. The trial was suspended several times, one of the reasons being severe side effects such as liver toxicity that could not be resolved. In all three of the deaths from the drug, the patients developed liver disease. In terms of drug dosage, the high-dose group reached a dose of 3E14 vg/kg, which corresponded to the injection of 300 trillion genomic fragments delivered by AAV vectors per kilogram of body weight. As a result, the AT132 faces enormous challenges, whether in terms of production difficulty, cost and expense, or potential safety.

Therefore, developing drugs with higher targeting and thus lower drug dosages, and developing drugs with better specificity and thus avoidance of adverse effects, are two main directions in which modification of AAV serotypes is being carried out.

### SUMMARY

In one aspect, the present application provides a mutant of an adeno-associated virus (AAV) capsid protein, compared with a VP1 capsid protein of wild-type AAV2, including any one or any combination of the following mutations:
(1) in variable region IV of the VP1 capsid protein, including an amino acid sequence KTINGSGQNQQTLK (SEQ ID NO: 2) or an amino acid sequence having 1, 2, 3, or 4 amino acid changes when compared to SEQ ID NO: 2;
(2) in variable region V of the VP1 capsid protein, including an amino acid sequence TTVTQ (SEQ ID NO: 3) or an amino acid sequence having 1 or 2 amino acid changes when compared to SEQ ID NO: 3; and
(3) replacement of amino acids at positions 585-587 with a short peptide comprising an RGD sequence,
where an amino acid sequence of the VP1 capsid protein of the wild-type AAV2 is set forth in SEQ ID NO: 1, and the positions of the amino acids correspond to positions in the amino acid sequence of the VP1 capsid protein of the wild-type AAV2.

In some embodiments, a sequence of the short peptide is SNSRGDYNSL (SEQ ID NO: 37), GPGRGDQTTL (SEQ ID NO: 38), or ENRRGDFNNT (SEQ ID NO: 39).

In some embodiments of the mutant of the AAV capsid protein, amino acids at positions 447-461 are replaced with the amino acid sequence KTINGSGQNQQTLK (SEQ ID NO: 2) or the amino acid sequence having 1, 2, 3, or 4 amino acid changes when compared to SEQ ID NO: 2, and the positions of the amino acids correspond to positions in the amino acid sequence of the VP1 capsid protein of the wild-type AAV2.

In some embodiments of the mutant of the AAV capsid protein, amino acids at positions 490-494 are replaced with the amino acid sequence TTVTQ (SEQ ID NO: 3) or the amino acid sequence having 1 or 2 amino acid changes when compared to SEQ ID NO: 3, and the positions of the amino acids correspond to positions in the amino acid sequence of the VP1 capsid protein of the wild-type AAV2.

In some embodiments, the mutant of the AAV capsid protein includes an amino acid sequence set forth in any one of SEQ ID NOs: 27-31 and 6-8, or an amino acid sequence having at least 80%, at least 90%, at least 95%, or at least 98% sequence identity to the amino acid sequence set forth in any one of SEQ ID NOs: 27-31 and 6-8.

In some embodiments, the mutant of the AAV capsid protein is a mutant of VP1 and/or VP2 and/or VP3 capsid protein.

In another aspect, the present application provides an isolated nucleic acid molecule encoding the mutant of the AAV capsid protein above.

In some embodiments, the isolated nucleic acid molecule includes an nucleotide sequence set forth in any one of SEQ ID NOs: 32-36 and 9-12.

In another aspect, the present application provides an expression vector including the isolated nucleic acid molecule above.

In another aspect, the present application provides a host cell including the isolated nucleic acid molecule above or the expression vector including the isolated nucleic acid molecule above.

In another aspect, the present application provides a host cell expressing the mutant of the AAV capsid protein above.

In another aspect, the present application provides an adeno-associated virus (AAV) including the mutant of the AAV capsid protein above.

In another aspect, the present application provides a method for preparing a recombinant adeno-associated virus (rAAV), including: introducing at least the following components into a host cell: (1) the isolated nucleic acid molecule above or an expression vector including the isolated nucleic acid molecule above; and (2) a GOI (gene of interest) plasmid including a target gene.

In some embodiments, an expression product of the target gene is protein or RNA.

In another aspect, the present application provides a rAAV prepared by the method above.

In some embodiments, the rAAV has lower targeting to liver than wild-type AAV2 or wild-type AAV9.

In some embodiments, the rAAV has higher targeting to muscle or heart than the wild-type AAV2 or wild-type AAV9.

In some embodiments, the present application provides a pharmaceutical composition including the rAAV above and a pharmaceutically acceptable carrier.

In another aspect, the present application provides a method for treating a disease, including: administering an isolated nucleic acid molecule encoding the mutant of the AAV capsid protein above, an expression vector, or a rAAV to a patient in need,

where the expression vector includes the isolated nucleic acid molecule encoding the mutant of the AAV capsid protein; and

the rAAV includes the mutant of the AAV capsid protein above.

In some embodiments, the disease is a muscle-related disease or a heart-related disease.

In some embodiments, the muscle-related disease is selected from the group consisting of muscular dystrophy, myasthenia gravis, polymyositis, dermatomyositis, and rhabdomyolysis; and the heart-related disease is selected from the group consisting of myocardial infarction, myocardial ischemia injury, coronary heart disease, myocardial hypertrophy, and myocardial fibrosis.

The protein mutants provided in this application enhance muscle targeting, thereby reducing drug cost and dosage. At the same time, the protein mutants provided in this application can reduce hepatotoxicity (or liver toxicity), improve specificity, avoid potential clinical application risks, and provide better gene therapy tools for patients or researchers. The protein mutants have great social value and economic benefit.

### BRIEF DESCRIPTION OF THE DRAWINGS

FIGS. 1A and 1B show analysis results of the targeting of different serotypes to livers of C57 mice (4 weeks after virus injection), where FIG. 1A shows the relative expression levels of mRNA in the livers; and FIG. 1B shows the relative expression levels of proteins in the livers.

FIGS. 2A, 2B, and 2C show analysis results of the targeting of different serotypes to quadriceps of C57 mice (4 weeks after virus injection), where FIG. 2A shows the relative expression levels of mRNA in the quadriceps; FIG. 2B shows a ratio of the mRNA level in the quadriceps to the mRNA level in the liver (i.e., quadriceps/liver ratio (mRNA expression levels)); and FIG. 2C shows the relative expression levels of proteins in the quadriceps.

FIGS. 3A, 3B, and 3C show analysis results of the targeting of different serotypes to hearts of C57 mice (4 weeks after virus injection), where FIG. 3A shows the relative expression levels of mRNA in the hearts; FIG. 3B shows a ratio of the mRNA level in the heart to the mRNA level in the liver (i.e., heart/liver ratio (mRNA expression levels)); and FIG. 3C shows the relative expression levels of proteins in the hearts.

FIGS. 4A and 4B show analysis results of the targeting of different serotypes to abdominal muscles of C57 mice (4 weeks after virus injection), where FIG. 4A shows the relative expression levels of mRNA in the abdominal muscles; and FIG. 4B shows a ratio of the mRNA level in the abdominal muscle to the mRNA level in the liver (i.e., abdominal muscle/liver ratio (mRNA expression levels)).

FIGS. 5A and 5B show analysis results of the targeting of different serotypes to brains of C57 mice (4 weeks after virus injection), where FIG. 5A shows the relative expression levels of mRNA in the brains; and FIG. 5B shows a ratio of the mRNA level in the brain to the mRNA level in the liver (i.e., brain/liver ratio (mRNA expression levels)).

FIGS. 6A and 6B show analysis results of the targeting of different serotypes to spinal cords of C57 mice (4 weeks after virus injection), where FIG. 6A shows the relative expression levels of mRNA in the spinal cords; and FIG. 6B shows a ratio of the mRNA level in the spinal cord to the mRNA level in the liver (i.e., spinal cord/liver ratio (mRNA expression levels)).

FIGS. 7A, 7B, and 7C show analysis results of the targeting of different serotypes to lungs, kidneys, and eyes of C57 mice (4 weeks after virus injection), where FIG. 7A shows the relative expression levels of mRNA in the lungs; FIG. 7B shows the relative expression levels of mRNA in the kidneys; and FIG. 7C shows the relative expression levels of mRNA in the eyes.

FIGS. 8A and 8B show analysis results of the tropism of different serotypes to mouse livers (4 weeks after virus injection), where FIG. 8A shows the relative expression levels of mRNA in the livers; and FIG. 8B shows the protein expression levels in the livers.

FIGS. 9A, 9B, 9C, 9D, 9E, and 9F show analysis results of the targeting of different serotypes to mouse muscles (quadriceps, biceps) (4 weeks after virus injection), where FIG. 9A shows the relative expression levels of mRNA in the quadriceps (quadriceps femoris); FIG. 9B shows a ratio of the mRNA level in the quadriceps to the mRNA level in the liver (i.e., quadriceps/liver ratio (mRNA expression levels)); FIG. 9C shows the protein expression levels in the quadriceps; FIG. 9D shows the relative expression levels of mRNA in the biceps (biceps brachii); FIG. 9E shows a ratio of the mRNA level in the biceps to the mRNA level in the liver (i.e., biceps/liver ratio (mRNA expression levels)); and FIG. 9F shows the protein expression levels in the biceps.

FIGS. 10A, 10B, 10C, 10D, 10E, and 10F show analysis results of the targeting of different serotypes to mouse abdominal muscles and hearts (4 weeks after virus injection), where FIG. 10A shows the relative expression levels of mRNA in the abdominal muscles; FIG. 10B shows a ratio of the mRNA level in the abdominal muscle to the mRNA level in the liver (i.e., abdominal muscle/liver ratio (mRNA expression levels)); FIG. 10C shows the protein expression levels in the abdominal muscles; FIG. 10D shows the relative expression levels of mRNA in the hearts; FIG. 10E shows a ratio of the mRNA level in the heart to the mRNA level in the liver (i.e., heart/liver ratio (mRNA expression levels)); and FIG. 10F shows the protein expression levels in the hearts.

FIGS. 11A and 11B show analysis results of the targeting of different serotypes to mouse brains and lungs (4 weeks after virus injection), where FIG. 11A shows the relative expression levels of mRNA in the brains; and FIG. 11B shows the relative expression levels of mRNA in the lungs.

FIGS. 12A, 12B, 12C, and 12D show the results of various biochemical indicators in cynomolgus monkeys (Macaca fascicularis) after injection of different serotypes, where FIG. 12A shows the relative change of ALT values; FIG. 12B shows the relative change of AST values; FIG. 12C shows the relative change of LDH values; and FIG. 12D shows the relative change of CK values. ALT: alanine aminotransferase; AST: aspartate aminotransferase; LDH: lactate dehydrogenase; CK: creatine kinase.

FIGS. 13A and 13B show analysis results of the tropism of different serotypes to livers of cynomolgus monkeys (Macaca fascicularis), where FIG. 13A shows the relative expression levels of DNA and mRNA in the livers; and FIG. 13B shows the protein expression levels in the livers.

FIGS. 14A, 14B, 14C, 14D, 14E, and 14F show analysis results of the targeting of different serotypes to muscles (gastrocnemii, quadriceps) of cynomolgus monkeys (14 days and 28 days after virus injection), where FIG. 14A shows the relative expression levels of DNA in the gastrocnemii; FIG. 14B shows the relative expression levels of mRNA in the gastrocnemii; FIG. 14C shows the protein expression levels in the gastrocnemii; FIG. 14D shows the relative expression levels of DNA in the quadriceps; FIG. 14E shows the relative expression levels of mRNA in the quadriceps; and FIG. 14F shows the protein expression levels in the quadriceps.

FIGS. 15A, 15B, 15C, 15D, 15E, and 15F show analysis results of the targeting of different serotypes to muscles (biceps, triceps) of cynomolgus monkeys (14 days and 28 days after virus injection), where FIG. 15A shows the relative expression levels of DNA in the biceps; FIG. 15B shows the relative expression levels of mRNA in the biceps; FIG. 15C shows the protein expression levels in the biceps; FIG. 15D shows the relative expression levels of DNA in the triceps (triceps brachii); FIG. 15E shows the relative expression levels of mRNA in the triceps; and FIG. 15F shows the protein expression levels in the triceps.

FIGS. 16A and 16B show analysis results of the tropism of different serotypes to mouse livers (4 weeks after virus injection), where FIG. 16A shows the relative expression levels of mRNA in the livers; and FIG. 16B shows the protein expression levels in the livers.

FIGS. 17A, 17B, and 17C show analysis results of the targeting of different serotypes to mouse muscles (quadriceps, abdominal muscles) and hearts (4 weeks after virus injection), where FIG. 17A shows the protein expression levels in the quadriceps; FIG. 17B shows the protein expression levels in the abdominal muscles; and FIG. 17C shows the protein expression levels in the hearts.

### DETAILED DESCRIPTION

Unless otherwise indicated, all technical and scientific terms used herein have the meanings commonly understood by those of ordinary skill in the art.

The term “or” refers to a single element of the enumerated optional elements, unless the context clearly indicates otherwise. The term “and/or” refers to any one, any two, any three, any more or all of the optional elements listed.

The terms “comprise” or “include” mean the inclusion of said elements, integers, or steps, but not the exclusion of any other elements, integers, or steps. When the term “comprise” or “include” is used herein, unless otherwise specified, the term also encompasses cases consisting of the recited elements, integers, or steps. For example, reference to a polypeptide “comprising” a particular sequence is intended to encompass polypeptides consisting of that particular sequence.

“Adeno-associated virus (AAV)” is a non-enveloped icosahedral capsid virus of the Parvoviridae family, including a viral genome of single-stranded DNA. The Parvoviridae family includes the genus Dependovirus, which includes AAV, and is dependent on the presence of a helper virus, such as adenovirus, for its replication. Due to its relatively simple structure, ability to infect a variety of cells (including quiescent and dividing cells) without integrating into the host genome, and its relatively mild immunogenicity, AAV has been demonstrated to be useful as a biological tool and for expressing a target gene in vitro or in vivo. Also considered herein are AAV-based expression vectors, including recombinant AAV (rAAV) carrying the target gene and used for therapeutic purposes.

The wild-type AAV viral genome is a linear, single-stranded DNA (ssDNA) molecule that is approximately 5,000 nucleotides (nt) in length. The AAV viral genome usually includes two inverted terminal repeat (ITR) sequences, which cap the viral genome at the 5′ and 3′ ends, respectively, and provide a replication origin for the viral genome. These ITRs have a characteristic T-shaped hairpin structure and have multiple functions, including but not limited to serving as an origin of DNA replication by acting as a primer for the endogenous DNA polymerase complex of the host cell during viral replication.

The wild-type AAV genome also includes the Rep gene and the Cap gene, which encode four non-structural Rep proteins (Rep78, Rep68, Rep52, and Rep40) and three capsid proteins or structural proteins (VP1, VP2, and VP3), respectively. The Rep protein is involved in viral replication and packaging, while capsid proteins are assembled to form the AAV protein shell or AAV capsid. Alternative splicing and alternate start codons and promoters result in the production of four different Rep proteins from a single open reading frame in the Rep gene and three capsid proteins from a single open reading frame in the Cap gene.

In the context of AAV, the term “viral capsid protein” or “capsid protein” is used to refer to the protein of AAV that is capable of self-assembling to produce AAV particles, and is also known as the coat protein or VP protein. The VP protein includes three subunits, VP1, VP2, and VP3, and thus changes in the mutant of the VP protein relative to the wild-type VP protein can be reflected in amino acid sequence changes in the VP1, VP2, and VP3 subunits. Accordingly, as used herein, “mutant of the (AAV) capsid protein” includes a mutant of a VP protein, but also includes a mutant of VP1, VP2, and/or VP3 subunit. Due to the consistency of amino acid sequences between VP1, VP2, and VP3 subunits expressed from the same Cap gene, when changes are made to the coding sequence in the Cap gene, for example, to the coding sequence of the VP3 subunit, the amino acid sequences of the expressed VP1 and VP2 subunits at the same time are altered.

The term “serotype” in the context of AAV is used to refer to the serological distinction of the capsid protein of AAV from other AAV serotypes. Serological uniqueness is determined based on the reactivity of an antibody with one AAV and the lack of cross-reactivity with other or another AAV. Such cross-reactivity differences are usually due to differences in capsid protein sequences (or subunit sequences thereof)/antigenic determinants (e.g., due to differences in VP1, VP2, and/or VP3 sequences of serotype AAV2). A variety of AAV serotypes have been identified, including but not limited to AAV1, AAV2, AAV3, AAV4, AAV5, AAV6, AAV7, AAV8, AAV9, AAV10, AAV11, and AAV12, as well as their mutants.

When referring to the capsid protein or its subunits of AAV, the term “variable region” refers to the region where the amino acid sequence varies relatively significantly between different serotypes. Usually, relatively conserved regions are identified by aligning amino acid sequences of capsid proteins from multiple AAV serotypes, and the sequences between the relatively conserved regions are the variable region sequences. The variable region may be associated with the binding of AAV to cell surface receptors. For the AAV2 serotype, multiple variable regions may be included, such as variable region I to variable region IX (or also referred to as loop I to loop IX). In a preferred embodiment, the positions of variable region IV and variable region V in the AAV2 capsid protein are determined according to the region determination method established by Bennett et al. (Bennett A, Keravala A, Makal V, et al. Structure comparison of the chimeric AAV2.7m8 vector with parental AAV2. J Struct Biol. 2020;209(2): 107433).

“Recombinant AAV vector” means an AAV genome derived by removing a portion of wild-type genes (e.g., the Rep gene and Cap gene) from the AAV genome using molecular biology methods and replacing them with heterologous nucleic acid sequences (e.g., coding sequences of proteins or RNAs used for therapeutic purposes). Typically, for a recombinant AAV vector, one or two inverted terminal repeat (ITR) sequences of the AAV genome are retained therein. Most often, recombinant AAV vectors are replication-deficient, lacking sequences encoding functional Rep and Cap proteins in their viral genomes. These replication-deficient AAV particles may lack most of the parental coding sequences and carry essentially only one or two AAV ITR sequences and target nucleic acids for delivery to a cell, tissue, organ, or organism. An AAV including a recombinant AAV vector is referred to herein as a recombinant AAV (rAAV).

The term “GOI (gene of interest) plasmid” as used herein refers to a plasmid that is introduced into a host cell together with a helper plasmid and/or a helper virus when preparing a recombinant AAV, and the GOI plasmid carries a target gene and ITR sequences located on both sides of the target gene. In order to facilitate the expression of the prepared recombinant AAV particles in vivo or in vitro, the target gene (a coding sequence for a protein or RNA) is usually operably linked to a regulatory sequence related to expression, such as a promoter and a polyadenylation (polyA) tailing signal. The term “operably linked” refers to a linkage between polynucleotide elements that places them in a functional relationship. When a nucleic acid or polynucleotide sequence is placed into a functional relationship with another nucleic acid sequence, they are “operably linked”. For example, a transcriptional regulatory sequence such as a promoter, an enhancer, or other expression control elements known in the art is operably linked to a coding sequence if it affects the transcription of the coding sequence.

“Amino acid changes” herein include amino acid substitutions, deletions or insertions. The number of amino acid changes in the mutant sequence relative to the parent sequence can be calculated as the sum of the number of amino acid substitutions, the number of amino acid deletions, and the number of amino acid insertions.

“A short peptide comprising an RGD sequence” means, as used herein, a short peptide of 3-10 amino acids in length including sequential arginine, glycine, and aspartic acid.

As used herein, the terms “nucleic acid molecule”, “nucleic acid”, and “polynucleotide” are used interchangeably to refer to a polymer of nucleotides. Such nucleotide polymers may contain natural and/or non-natural nucleotides and include, but are not limited to, DNA, RNA, and peptide nucleic acids (PNA). The term “nucleic acid sequence” refers to a linear sequence of nucleotides contained in a nucleic acid molecule or polynucleotide. The term “isolated nucleic acid molecule” refers to a nucleic acid molecule that is separated from its natural environment (such as the intracellular environment) and is essentially free of one or more substances that are normally associated with it in nature, such as proteins, nucleic acids, lipids, carbohydrates, cell membranes, etc., or is an artificially prepared (such as artificially synthesized) nucleic acid molecule.

The term “expression vector” refers to a nucleic acid molecule including various expression elements for expressing a target protein or target RNA in a host cell. For expression vectors used to express target proteins in eukaryotic cells, these expression elements generally include promoters, enhancers, polyadenylation signal sequences, and the like. In order to facilitate amplification in E. coli, the expression vector usually also includes an E. coli replicon sequence. In addition, the expression vector may also include an antibiotic resistance gene or a selection marker gene for screening (e.g., ampicillin resistance gene (AmpR), thymidine kinase gene (TK), kanamycin resistance gene (KanR), neomycin resistance gene (NeoR), etc.) and a multiple cloning site (MCS) for inserting the target gene.

The term “host cell” refers to a cell in which an expression vector can be maintained and/or replicated, including a prokaryotic cell and a eukaryotic cell, such as a bacterium (such as E. coli), a fungus (yeast), an insect cell (such as SF9) and a mammalian cell (such as HEK-293T).

When referring to a pharmaceutical composition, the term “pharmaceutically acceptable carrier” refers to a solid or liquid diluent, filler, antioxidant, stabilizer, or other substances that can be safely administered. These substances are suitable for administration to humans and/or animals without undue adverse side effects while being suitable for maintaining the activity of the drug or active agent located therein. According to the route of administration, various different carriers well known in the art can be administered, including, but not limited to, sugars, starch, cellulose and its derivatives, maltose, gelatin, talc, calcium sulfate, vegetable oils, synthetic oils, polyols, alginic acid, phosphate buffer, emulsifiers, isotonic saline, and/or pyrogen-free water, etc.

The “targeting” or “tropism” of AAV or rAAV refers to the phenomenon that when AAV or rAAV is introduced into the body, it relatively accumulates in a specific tissue or organ. For example, targeting or tropism can be manifested as a higher concentration in tissue A than in tissue B. This targeting or tropism can be reflected by detecting the content or concentration of its genome in different tissues or organs.

When referring to amino acid or nucleotide sequences, the term “sequence identity” (also known as “sequence consistency”) refers to a measure of the degree of identity between two amino acid or nucleotide sequences (e.g., a query sequence and a reference sequence), generally expressed as a percentage. Typically, before calculating the percent identity between two amino acid or nucleotide sequences, the sequence alignment is first conducted and gaps (if any) are introduced. If the amino acid residues or bases in the two sequences are the same at a certain alignment position, the two sequences are considered to be consistent or matched at that position; if the amino acid residues or bases in the two sequences are different, they are considered to be inconsistent or mismatched at that position. In some algorithms, the number of matching positions is divided by the total number of positions in the alignment window to obtain sequence identity. In other algorithms, the number of gaps and/or the length of the gaps are also taken into account. Commonly used sequence alignment algorithms or software include DANMAN, CLUSTALW, MAFFT, BLAST, MUSCLE, etc. For the purpose of this application, the publicly available alignment software BLAST (available from https://www.ncbi.nlm.nih.gov/) can be used to obtain the best sequence alignment and calculate the sequence identity between two amino acid or nucleotide sequences using the default settings.

The present application is based, at least in part, on the finding that substitution of a partial sequence of the AAV2 capsid protein (hereinafter referred to as the “substituted sequence”) results in a mutant of the capsid protein with altered targeting to an organ, and in particular, reduced liver tropism. The sequence used for substitution (hereinafter referred to as the “substitution sequence”, i.e., a “substituted sequence” is replaced with a “substitution sequence”) may be derived from capsid proteins of other serotypes. In some embodiments, the substitution sequence used is from the AAV9 capsid protein. In some embodiments, the substitution sequence is KTINGSGQNQQTLK (SEQ ID NO: 2). In other embodiments, the substitution sequence is an amino acid sequence having 1, 2, 3, or 4 amino acid changes when compared to SEQ ID NO: 2. In some embodiments, the substitution sequence is TTVTQ (SEQ ID NO: 3). In other embodiments, the substitution sequence is an amino acid sequence having 1 or 2 amino acid changes when compared to SEQ ID NO: 3. In some embodiments, sequence substitutions are performed at two or more positions of the capsid protein of AAV2. In some embodiments, the substituted sequence is located in the variable region IV. In other embodiments, the substituted sequence is located in the variable region V. In some embodiments, the substituted sequence is located in the variable region IV and the variable region V. Preferably, the sequence SEQ ID NO: 4 of the capsid protein of wild-type AAV2 may be replaced with a substitution sequence SEQ ID NO: 2 or an amino acid sequence having 1, 2, 3, or 4 amino acid changes when compared to SEQ ID NO: 2. Preferably, the sequence SEQ ID NO: 5 of the capsid protein of wild-type AAV2 can be replaced with the substitution sequence SEQ ID NO: 3 or the amino acid sequence having 1 or 2 amino acid changes when compared to SEQ ID NO: 3. More preferably, the sequence SEQ ID NO: 4 of the capsid protein of wild-type AAV2 can be replaced with the substitution sequence SEQ ID NO: 2 or the amino acid sequence having 1, 2, 3, or 4 amino acid changes when compared to SEQ ID NO: 2, and the sequence SEQ ID NO: 5 of the capsid protein of wild-type AAV2 can be replaced with the substitution sequence SEQ ID NO: 3 or the amino acid sequence having 1 or 2 amino acid changes when compared to SEQ ID NO: 3. Most preferably, the sequence SEQ ID NO: 4 of the capsid protein of the wild-type AAV2 can be replaced with the substitution sequence SEQ ID NO: 2, and the sequence SEQ ID NO: 5 of the capsid protein of wild-type AAV2 can be replaced with the substitution sequence SEQ ID NO: 3.

The present application also finds that the introduction of a short peptide including an RGD sequence into the amino acid sequence of the AAV2 capsid protein improves its targeting to liver and/or increases its targeting to muscle or heart. In some embodiments, the short peptide including the RGD sequence is SNSRGDYNSL (SEQ ID NO: 37). In other embodiments, the short peptide including the RGD sequence is GPGRGDQTTL (SEQ ID NO: 38). In other embodiments, the short peptide including the RGD sequence is ENRRGDFNNT (SEQ ID NO: 39). Preferably, the 3 amino acids (e.g., RGN sequence) from position 585 of the AAV2 capsid protein (VP1, VP2 and/or VP3) are replaced with the short peptide including the RGD sequence above.

Simultaneous introduction of the substitution sequence and the short peptide including the RGD sequence above into the capsid protein of AAV2 results in excellent targeting to muscle or heart as well as reduced targeting to liver.

When referring to specific amino acid positions herein, they are all positions corresponding to positions in the amino acid sequence of the wild-type VP1 protein set forth in SEQ ID NO: 1. The corresponding positions can be determined by simple alignment of amino acid sequences.

Accordingly, the present application provides the following embodiments.

A mutant of an AAV capsid protein having muscle targeting and low hepatotoxicity, wherein:
(a) An amino acid sequence of the mutant is a sequence set forth in any one of SEQ ID NOs: 27-31;
(b) the mutant is a protein derived from the amino acid sequence in (a) by substitution, deletion, or addition of one or more amino acids, while possessing the activity of the amino acid sequence in (a).

A recombinant adeno-associated virus virion, including:
(a) the mutant of the AAV capsid protein above; and
(b) a heterologous polynucleotide encoding a heterologous gene product.

In some embodiments, the heterologous gene product is a polypeptide.

In other embodiments, the heterologous gene product is selected from the group consisting of an interfering RNA, an aptamer, an endonuclease, and a guide RNA.

Also provided herein is the use of the above-mentioned recombinant adeno-associated virus virion in the preparation of a medicament for delivering a gene product to a cell or a tissue of a subject.

In some embodiments, the tissue is selected from a muscle or heart tissue.

The present application also provides the use of the recombinant adeno-associated virus virion in the preparation of a medicament for preventing or treating a muscle-related disease or a heart-related disease.

In some embodiments, the muscle-related disease is selected from the group consisting of muscular dystrophy, myasthenia gravis, polymyositis, dermatomyositis, and rhabdomyolysis; and the heart-related disease is selected from the group consisting of myocardial infarction, myocardial ischemia injury, coronary heart disease, myocardial hypertrophy, and myocardial fibrosis

The present application will be further described below in conjunction with specific embodiments, and the advantages and features of the present application will become clearer as the description proceeds. These embodiments are merely exemplary and do not limit the scope of the present application in any way. Those skilled in the art should understand that the details and forms of the technical solutions of the present application may be modified or replaced without departing from the spirit and scope of the present application, but these modifications and replacements are within the scope of the present application.

### EXAMPLE 1: DESIGN, CONSTRUCTION, AND VIRUS PRODUCTION OF AAV MUTANTS (OR AAV VARIANTS)

### (1) Design of AAV Mutants (Or AAV Variants):

The inventors analyzed the three-dimensional structures of AAV2 (PDB: 6IH9) and AAV9 (PDB: 3UX1) and literature data, and replaced the key sites related to targeting binding with an AAV2 receptor, such as IV loop (AAV2: R447-Q461 replaced by AAV9: K449-K462) and V loop (AAV2: K490-D494 replaced by AAV9: T491-Q495), to obtain mutant 1′ of the target serotype (SEQ ID NO: 6). Since R585 of AAV2 is the key amino acid site for binding to heparan sulfate proteoglycan (HSPG), and HSPG is one of the main receptors mediating the liver tropism of AAV2, the inventors further mutated this to form mutant 2′ (AAV2: R585 mutated to A585) (SEQ ID NO: 7) and mutant 3′ (AAV2: deletion mutation of R585GN) (SEQ ID NO: 8), and further conducted relevant in vivo activity tests in animals.

### (2) Construction of Mutant Serotype Vector and Plasmid Extraction:

The Rep-CAP plasmid was double-digested with SmiI and BshTI, subjected to gel electrophoresis, and a fragment band of about 5000 bp was cut out for gel recovery to obtain a digested backbone fragment.

According to the Cap sequence of mutant 1′, the following primers were designed, and the specific steps included: The Rep-CAP plasmid of AAV2 was used as a template and the primers Cap-f+YJ69-R were used for amplification; then the amplified product was recovered by gel extraction to obtain the target product 246-1. The Rep-CAP plasmid of AAV2 was used as a template and primers YJ69-F+YJ72-R were used for amplification; then the amplified product was recovered by gel extraction to obtain the target product 246-2. The Rep-CAP plasmid of AAV2 was used as a template and YJ72-F+cap-r primers were used for amplification; then the amplified product was recovered by gel extraction to obtain the target product 246-3. There are homology arm sequences between the backbone fragment and the fragment and between the fragments, and multiple fragments can be assembled into a complete vector through Gisbon. The Rep-CAP plasmid of mutant 1′ can be recombined and constructed by mixing the backbone fragment, 246-1, 246-2, and 246-3 in the following steps and proportions.

According to the Cap sequence of mutant 2′, the following primers were designed, and the specific steps included: The Rep-CAP plasmid of AAV2 was used as a template and the primers Cap-f+YJ69-R were used for amplification; then the amplified product was recovered by gel extraction to obtain the target product 246-1. The Rep-CAP plasmid of AAV2 was used as a template and primers YJ69-F+YJ72-R were used for amplification; then the amplified product was recovered by gel extraction to obtain the target product 246-2. The Rep-CAP plasmid of AAV2 was used as a template and primers YJ72-F+247-R were used for amplification; then the amplified product was recovered by gel extraction to obtain the target product 247-3. The Rep-CAP plasmid of AAV2 was used as a template and primers 247-F+cap-r were used for amplification; then the amplified product was recovered by gel extraction to obtain the target product 247-4. There are homology arm sequences between the backbone fragment and the fragment and between the fragments, and multiple fragments can be assembled into a complete vector through Gisbon. The Rep-CAP plasmid of mutant 2′ can be recombined and constructed by mixing the backbone fragment, 246-1, 246-2, 247-3, and 247-4 in the following steps and proportions.

According to the Cap sequence of mutant 3′, the following primers were designed, and the specific steps included: The Rep-CAP plasmid of AAV2 was used as a template and the primers Cap-f+YJ69-R were used for amplification; then the amplified product was recovered by gel extraction to obtain the target product 246-1. The Rep-CAP plasmid of AAV2 was used as a template and primers YJ69-F+YJ72-R were used for amplification; then the amplified product was recovered by gel extraction to obtain the target product 246-2. The Rep-CAP plasmid of AAV2 was used as a template and primers YJ72-F+248-R were used for amplification; then the amplified product was recovered by gel extraction to obtain the target product 248-3. The Rep-CAP plasmid of AAV2 was used as a template and primers 248-F+cap-r were used for amplification; then the amplified product was recovered by gel extraction to obtain the target product 248-4. There are homology arm sequences between the backbone fragment and the fragment and between the fragments, and multiple fragments can be assembled into a complete vector through Gisbon. The Rep-CAP plasmid of mutant 3′ can be recombined and constructed by mixing the backbone fragment, 246-1, 246-2, 248-3, and 248-4 in the following steps and proportions.

The primers involved in the construction of the Rep-CAP vector of mutants 1′-3′ of the AAV capsid protein in this application are:

| Primer name | Primer sequence (5′ → 3′) |
|---|---|
| Cap-f | CATCTTTGAACAATAAATGATTTAAATCAGGTATG (SEQ ID NO: 13) |
| cap-r | TCAACTGAAACGAATCAACCGGTTT (SEQ ID NO: 14) |
| YJ69-R | TGTTGATTCTGTCCAGAACCGTTAATAGTCTTGCTCAAGTAATACAGGTACTGGTC |
| | GAT (SEQ ID NO: 15) |
| YJ69-F | ACGGTTCTGGACAGAATCAACAAACGCTAAAATTTTCTCAGGCCGGAGCGAGT |
| | (SEQ ID NO: 16) |
| YJ72-R | GTTGTTTTGAGTCACAGTGGTTGATACTCGCTGCTGGCGGTAACAG (SEQ ID NO: |
| | 17) |
| YJ72-F | ATCAACCACTGTGACTCAAAACAACAACAGTGAATACTCGTGGACTGGAG (SEQ |
| | ID NO: 18) |
| 247-R | TTGTCTGTTGCCAGCCTGGAGGTTGGTAGATACAGAACCATACT (SEQ ID NO: 19) |
| 247-F | CCTCCAGGCTGGCAACAGACAAGCAGCTACCGCAGAT (SEQ ID NO: 20) |
| 248-R | CTGCTTGTCTCTGGAGGTTGGTAGATACAGAACCATACT (SEQ ID NO: 21) |
| 248-F | CAACCTCCAGAGACAAGCAGCTACCGCAGAT (SEQ ID NO: 22) |

One clean PCR tube with a volume of 200 μL was taken, labeled, and placed on an ice box. The reaction solution was prepared with the digested backbone fragment above and each target fragment (i.e., the target product above). In the reaction solution, the molar ratio of the digested backbone fragment to each target fragment was 1:3. The reaction solution was reacted in a PCR instrument at 50° C. for 30 min to perform recombination ligation. 50 μL of competent cells were thawed on ice, and 10 μL of ligation product was mixed with DH5α competent cells. The resulting mixture was placed on ice for 20-30 min; heat-shocked at 42° C. for 45 seconds; quickly placed in an ice bath on ice for 2 min, and then added to 400 μL of SOC medium (without antibiotics) for recovery and incubated at 37° C., 200 rpm for 1 h. The incubated product was evenly spread on an Amp-resistant plate (50 μg/ml) and cultured at 37° C. for 14 hours. The monoclonal bacteria were selected and expanded in 4 mL of liquid LB medium (Amp+ resistance) for 14 hours at 37° C.

The bacterial solution was centrifuged at 12,000 rpm for 1 min and the supernatant medium was poured off to obtain an EP tube containing the bacterial precipitate. 250 μL of a mixture of buffer P1/RNaseA was added to the EP tube and the bacteria were resuspended through vortex at a high speed. Subsequently, 250 μL of buffer P2 was added to the EP tube and then the EP tube was turned up and down 8-10 times. Then, 350 μL of buffer P3 was added to the EP tube, and the solution was immediately inverted and mixed 8-10 times to allow for thorough neutralization. The EP tube was centrifuged at 13,000 rpm for 10 minutes, and the resulting supernatant was passed through the column. The centrifuge tube containing the resulting column was centrifuged at 12,000 rpm for 1 min and the waste liquid was discarded. 500 μL of PW1 was added to the centrifuge tube, the resulting centrifuge tube was centrifuged at 12,000 rpm for 1 min, and the waste liquid was discarded. 600 μL of PW2 was added to the centrifuge tube, the obtained centrifuge tube was centrifuged at 12,000 rpm for 1 min, and the supernatant was poured off. 600 μL of PW2 was added to the centrifuge tube, the resulting centrifuge tube was centrifuged at 12,000 rpm for 1 min, and the supernatant was poured off. After the centrifuge tube was centrifuged without load at 12,000 rpm for 2 min, 30-50 μL of eluent pre-heated at 55° C. was added, the centrifuge tube was allowed to stand for 2 min, and centrifuged at 12,000 rpm for 1 min. Concentration detection was performed using a micro nucleic acid quantifier.

The obtained plasmid was tested for concentration, and 10 μL of the positive plasmid identified by enzyme digestion was taken for sequencing, and the positive plasmid was stored at −20° C. The sequencing results show that the obtained plasmid could encode a mutant capsid protein VP1. Finally, according to the amount of virus required for later testing, the relevant helper plasmids, each group of Rep-Cap plasmids (AAV2, AAV9, and mutants in this application) and GOI plasmids (ssAAV.CAG.Fluc-2a-eGFP.WPRE.SV40pA) were extracted.

### (3) Packaging and Purification of Mutants of Virus Serotypes

The Rep-Cap plasmids of each group (AAV2, AAV9 and the mutants of AAV of the present application), a plasmid expressing firefly luciferase (Fluc) and green fluorescent protein (EGFP), and a pHelper plasmid were co-transfected into an HEK-293T cell in an appropriate amount, and then the AAV viruses were purified by iodixanol gradient ultracentrifugation. The measured virus titer was 1E+12GC/mL-1E+13GC/mL, which was a suitable titer. The obtained AAV viruses were stored at −80° C. for future use.

### EXAMPLE 2: COMPARATIVE TEST OF VARIOUS INDICATORS OF MUTANT SEROTYPES

### (1) Animal Injection and Dissection

Animal experiments were performed using 6-8 week old C57 male mice. The relevant viruses were formulated according to the designed experimental and control groups. Each group was injected with 1E12GC viruses per mouse. Animal dissection and sampling of various organs were performed 4 weeks after injection. Samples were snap frozen in liquid nitrogen immediately after sampling and used for subsequent experiments such as RNA extraction and western blot (WB) detection, respectively.

(2) Detection of mRNA Expression Levels of Target Genes

### (2.1) Total RNA Extraction and Reverse Transcription:

Sample grinding: The grinder was pre-cooled 10 min in advance and the grinding parameters were set. An animal tissue sample stored in a −80° C. refrigerator was taken out. About 50-100 mg of the tissue was taken from the sample, clipped to the size of soybean grains in a sterile petri dish, then transferred to a 1.5 mL RNase-free EP tube after clipping the tissue to the size of soybean grains in a sterile petri dish. At the ratio of 1 mL of TransZol Up per 50-100 mg of the tissue, an appropriate amount of TransZol Up was added to the EP tube, two clean and sterile steel grinding balls with a diameter of 3 mm were then added, and the tube was sealed with parafilm. The sample was placed and leveled in a 24-well grinding adapter, the screw was tightened, and the cap-off button was pressed. The grinding program was initiated, after the program was finished, the sample was taken out and the particle size of the sample after grinding was observed; if no large piece of the tissue remained, the subsequent extraction operation could be carried out. After grinding, the sample was centrifuged at 12,000×g and 4° C. for 2 min, and the supernatant was transferred to a new RNase-free 1.5-mL eppendorf (EP) tube labeled accordingly.

Extraction of total RNA from a sample: For details, refer to the instruction manual of TransZol Up Plus RNA Kit (Beijing TransGen Biotech Co., Ltd., Item No.: ER501). Whenever 1 ml of TranZol Up was used, 0.2 mL of the RNA extraction agent was added to the sample and the resulting mixture was shaken vigorously for 5 min; centrifugation was performed at 12,000×g and 4° C. for 10 min. At this point, the sample was divided into three layers, and the colorless aqueous phase was transferred to a new RNase-free 1.5-mL EP tube, to which an equal volume of anhydrous ethanol was added (precipitation may occur at this point), and the EP tube was gently inverted to mix evenly. The obtained solution together with the precipitate was added to the spin column, and the spin column was centrifuged at 12,000×g for 30 seconds at room temperature, and the filtrate was discarded. 500 μL of CB9 was added to the spin column, which was then centrifuged at 12,000×g for 30 seconds at room temperature, and the filtrate was discarded; the previous step was repeated once. 500 μL of WB9 was added to the spin column, which was then centrifuged at 12,000×g for 30 seconds at room temperature, and the filtrate was discarded; the previous step was repeated once. The spin column was centrifuged at 12,000×g for 2 min at room temperature to completely remove residual ethanol. The spin column was placed into a RNase-free 1.5-mL EP tube and 30-50 μL (depending on tissue size) of RNase-free water was added to the center of the spin column and allowed to stand for 1 min at room temperature. The EP tube containing the spin column was centrifuged at 12,000×g for 1 min at room temperature to elute RNA.

Determination of nucleic acid concentration in the sample: The RNA concentration was detected using the micro nucleic acid quantifier, the concentration, OD260/280, and OD260/230 were recorded, and the RNA was stored at −80° C.

Reverse transcription: EasyScript® All-in-One First-Strand cDNA Synthesis SuperMix for qPCR (One-Step gDNA Removal) (Beijing TransGen Biotech Co., Ltd., Item No.: AE341-03) was used for each group of RNA samples, and the specific steps were described in the instruction manual.

(2.2) Quantitative PCR (qPCR) Experiments:

Each group of cDNA was used as a template and the qPCR system was prepared according to the instructions of 2× SYBR Green qPCR Master Mix (BIMAKE, Item No.: B21203):

### qPCR System

| Reagent | Usage | |
|---|---|---|
| 2× SYBR Green qPCR Master Mix | 10 | μL |
| cDNA template | 1.5 | μL |
| Upstream primer (10 μM) | 1 | μL |
| Downstream primer (10 μM) | 1 | μL |
| ROX Reference Dye | 0.4 | μL |

| | |
|---|---|
| Deionized water | Up to 20 μL |

| Primer name | Primer sequence (5′ → 3′) |
|---|---|
| Fluc2-qPCR-F1 | AACCAGCGCCATTCTGATCA (SEQ ID NO: 23) |
| Fluc2-qPCR-R1 | TCGGGGTTGTTAACGTAGCC (SEQ ID NO: 24) |
| GAPDH-F2 | CAGGAGAGTGTTTCCTCGTCC (SEQ ID NO: 25) |
| GAPDH-R2 | TTCCCATTCTCGGCCTTGAC (SEQ ID NO: 26) |

### Settings of qPCR Procedure

| | 1 | | | | | | | | | | | |
|---|---|---|---|---|---|---|---|---|---|---|---|---|
| | Hot-Start | | | | | | | | | | | |
| | DNA | | 2 | | | | | | | | | |
| | Polymerase | | PCR | | | | 3 | | | | | |
| | Activation | | Cycle (40 cycles) | | | | Melt Curve | | | | | |
| Step | Hold | | Denature | | Anneal/Extend | | Cycle (1 cycle) | | | | | |
| Temp | 95.0° | C. | 95.0° | C. | 60.0° | C. | 95.0° | C. | 60.0° | C. | 95° | C. |
| Time | 10 | min | 15 | sec | 30 | sec | 15 | sec | 60 | sec | 15 | sec |

### (2.3) Data Analysis

Relative expression was calculated from the Ct values of each group according to the formula 2\hat{ }-AAct.

### (3) WB Detection of Expression Levels of Target Proteins

Sample pre-treatment: The tissue was cut into tiny pieces and weighed. The weight of the tissue was recorded. The tiny pieces were placed in a 1.5 mL or 2 mL centrifuge tube; and the centrifuge tube was labeled and frozen at −80° C. for use. The cryo-grinder was pre-chilled. RIPA (Shanghai Beyotime Biotechnology Co., Ltd., P0013B) lysate was solubilized (Phenylmethylsulfonyl fluoride (PMSF) was added within a few minutes prior to use to obtain a final PMSF concentration of 1 mM). The above complete lysate was added to the centrifuge tube at a ratio of 150-250 μL of lysate per 20 mg of the tissue. Then two sterilized zirconium oxide grinding balls were added to the centrifuge tube and the sample was ground directly in the lysis buffer (the tissue sample of the brain or spinal cord: The temperature was −20° C., and the frequency was 70 Hz; the oscillation time was 50 seconds followed by a pause of 10 seconds, and this cycle was repeated 3-4 times; and the sample of the muscle or liver: The temperature was −20° C., and the frequency was 70 Hz; the oscillation time was 50 seconds followed by a pause of 10 seconds, and this cycle was repeated 5-7 times). After grinding, the sample was centrifuged in a freezing centrifuge at 4° C., 12,000×g for 5-10 min, and then the supernatant was transferred to a new sterilized EP tube and stored at −20° C. or −80° C.

Determination of protein concentration: Protein concentration was determined according to the method in the protein concentration assay kit based on the modified BCA method (Sangon Biotech (Shanghai) Co., Ltd., Item No.: C503051). Then, an appropriate amount of a sample of homogenate protein was taken according to the required amount, mixed with the corresponding amount of 5× SDS-PAGE protein loading buffer, boiled in a water bath for 10 min, cooled and centrifuged at a low speed for a while, and waited for sample loading.

Western blot (WB) detection:

SDS-PAGE electrophoresis: The appropriate loading amount was determined by the protein concentration and expression level, and was less than 20 μL/well. The loading amount of the tissue homogenate protein was about 20-50 μg. The specific operation process of electrophoresis was as follows: The comb on the precast gel was pulled out and the gel was mounted in the electrophoresis chamber. The electrophoresis buffer was added to the inner and outer chambers, where the inner chamber was filled with a freshly prepared buffer and checked for leaks. If there was no leakage in the inner chamber, then electrophoresis buffer could be added to the outer chamber. An appropriate amount of a treated protein sample was taken for loading, the pre-stained standard protein was used as a reference, and electrophoresis was carried out on a Tanon (Shanghai tanon life science Co., Ltd.) electrophoresis device at a constant voltage of 100 V for 100 min until the bromophenol blue reached the bottom of the gel. The power was turned off, the precast gel plate was carefully unloaded, and the gel was removed and placed in the transfer buffer to await subsequent manipulation.

Transfer: Six sheets of filter paper and one sheet of PVDF membrane were cut according to the gel area. The PVDF membrane was soaked in methanol for 5-10 seconds and then transferred to and soaked in the transfer buffer for 5 min, and the filter paper was also pre-wetted in the transfer buffer. Installation of the transfer device: Negative electrode (black plate)—sponge—3 layers of moistened filter paper—gel—PVDF membrane—3 layers of moistened filter paper—sponge—positive electrode (transparent plate). Each layer of air bubbles in the transfer device was driven away to avoid affecting the transfer effect, and after clamping the bracket, the transfer device was placed into the electrotransfer tank. The membrane was transferred in an ice bath at a constant voltage of 100 V for 100 min. The success of membrane transfer was determined by whether protein bands of the pre-stained molecular weight marker were completely transferred to the PVDF membrane. The transferred PVDF membrane was soaked and washed in PBST solution at room temperature for 5 min, and the PVDF membrane was cut according to the requirements. Be careful not to let the PVDF membrane dry out during the cutting process.

Blocking and antibody incubation: The PVDF membrane was incubated with a blocking solution (5% skimmed milk powder) for 2 hours at room temperature or overnight at 4° C. The blocked PVDF membrane was transferred to a primary antibody hybridization solution and incubated for 1 h at room temperature or overnight at 4° C. Luciferase Rabbit Polyclonal antibody (Proteintech Group, Inc., 27986-1-AP) was added to 4 mL of QuickBlock™ Western primary antibody dilution (Shanghai beyotime biotechnology Co., Ltd., P0256) in a ratio of 1:2000 to prepare the primary antibody hybridization; GADPH Rabbit Polyclonal antibody (Proteintech Group, Inc., 10494-1-AP) was added to 4 mL of QuickBlock™ Western primary antibody dilution (Shanghai beyotime biotechnology Co., Ltd., P0256) in a ratio of 1:2000 to prepare the primary antibody hybridization; and Rabbit GFP tag Polyclonal antibody (Proteintech Group, Inc., 50430-2-AP) was added to 4 mL of QuickBlock™ Western primary antibody dilution (Shanghai beyotime biotechnology Co., Ltd., P0256) in a ratio of 1:2000 to prepare the primary antibody hybridization. Then the membrane was washed three times with PBST for 5 min each time. The washed PVDF membrane was transferred into the secondary antibody hybridization solution and incubated at room temperature for 1 h. HRP-conjugated Affinipure Goat Anti-Rabbit IgG (H+L) (Proteintech Group, Inc., SA00001-2) was added to 4 mL of QuickBlock™ Western Secondary Antibody Diluent (Shanghai beyotime biotechnology Co., Ltd., P0258) at a ratio of 1:5000 to obtain the secondary antibody hybridization solution. The membrane was washed three times by PBST for 5 min each time.

Color development: ECL chemiluminescence kit's liquid A and liquid B were mixed in an equal volume. After shaking and mixing, the luminescent liquid was dropped on the PVDF membrane such that all the PVDF membrane was covered with the luminescent liquid, and the exposure time was adjusted such that the protein bands were clear, and then photographed by the instrument.

### (4) Results:

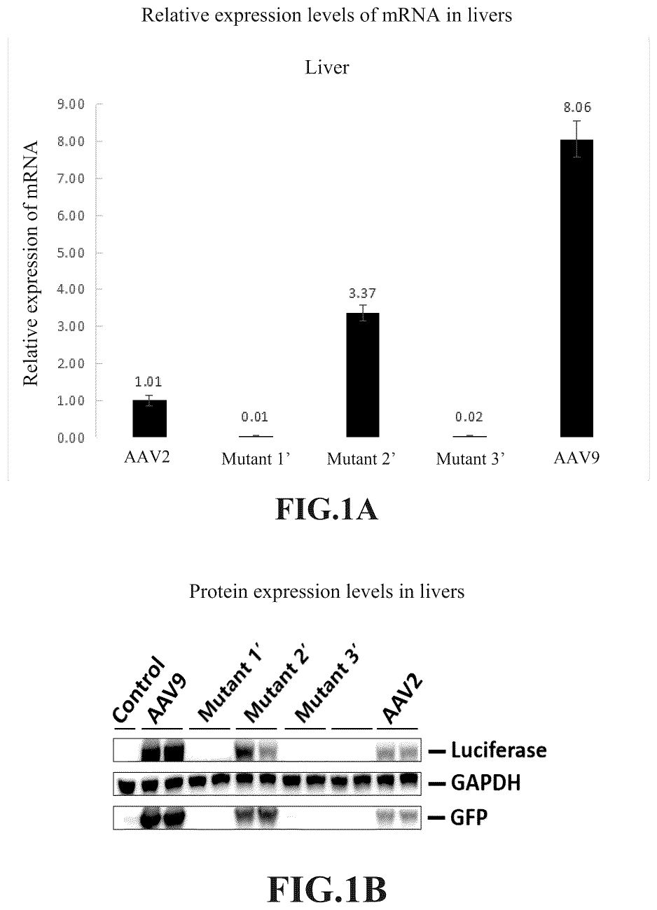

Comparing the above different modified mutants with AAV2 and AAV9 controls, it can be found that the three modified mutants have the characteristics of low liver tropism. The liver mRNA levels of mutant 1′, mutant 2′, and mutant 3′ (FIG. 1A) are 806-fold, 2.39-fold, and 403-fold lower than that of AAV9, respectively. The liver mRNA levels of mutants 1′ and 3′ are 101-fold and 50.5-fold lower than that of AAV2. Detection of target protein levels (firefly luciferase and eGFP) (FIG. 1B) also confirms the characteristic of low liver tropism. In addition, we have also detected and analyzed the targeting to different tissues and organs.

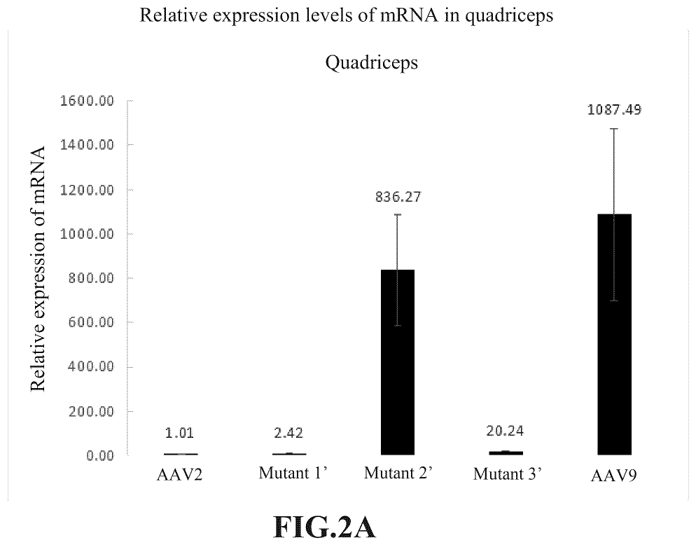

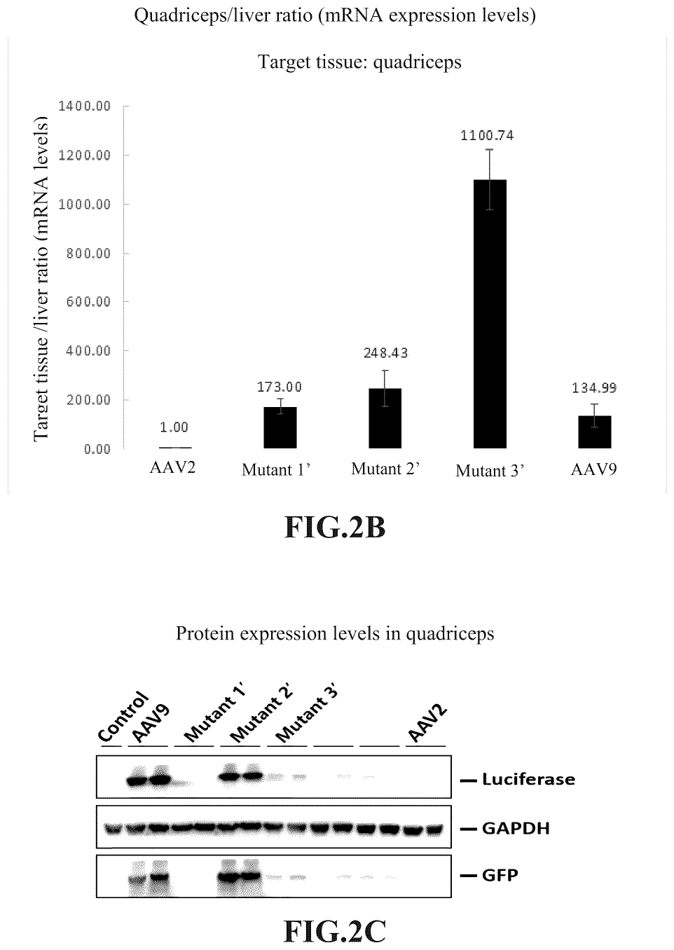

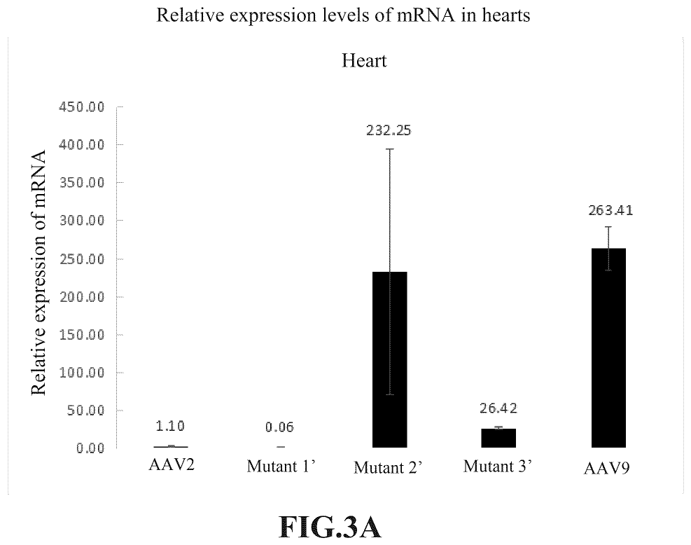

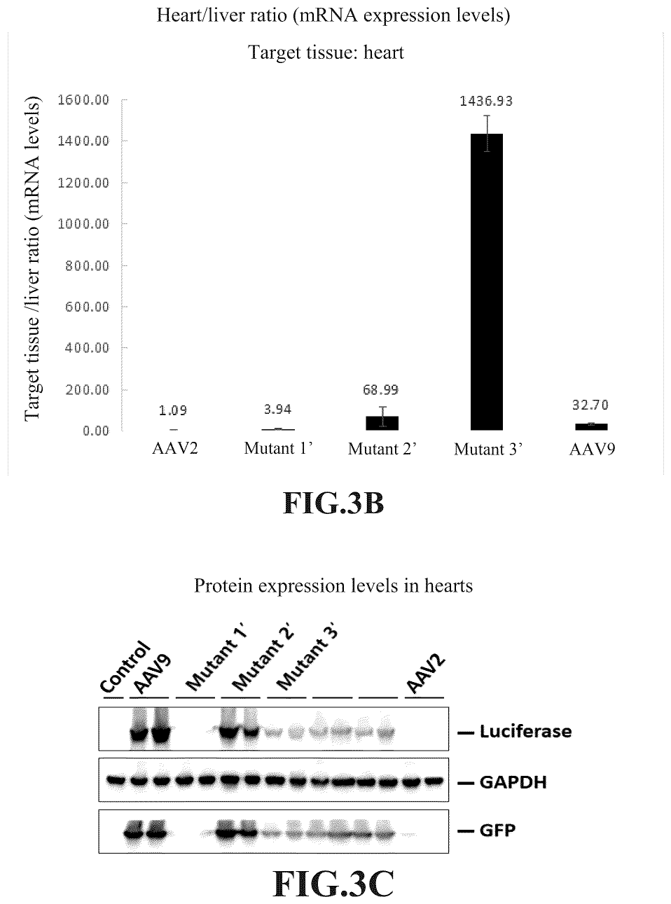

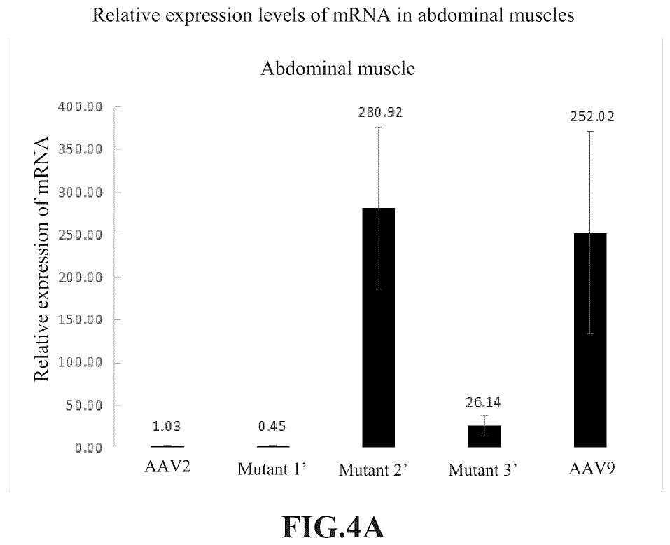

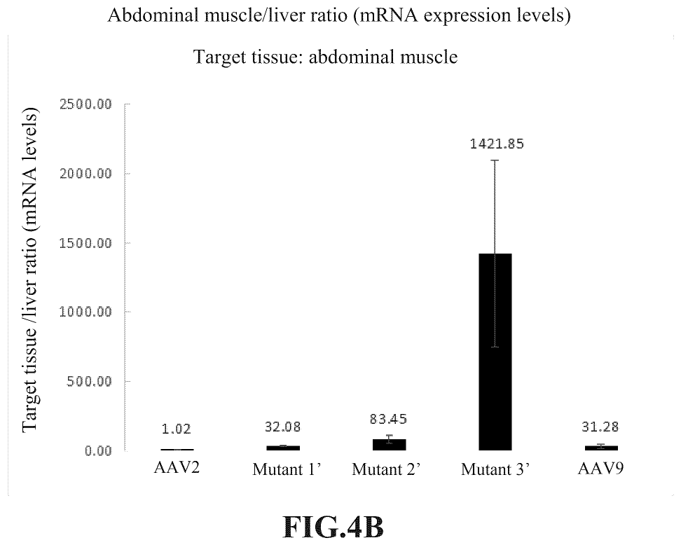

For the targeting to muscle and heart, mutant 2′ and mutant 3′ have higher infectious ability to quadriceps, abdominal muscles, and hearts than AAV2 (regarding the comparison of mRNA levels, 836-fold and 20-fold for quadriceps (FIG. 2A), 280-fold and 26-fold for abdominal muscles (FIG. 4A), and 232-fold and 26-fold for hearts (FIG. 3A)). Moreover, mutant 2′ is even closer to AAV9 in terms of mRNA and protein levels, and the ratio of mRNA levels in the target tissues to those in the liver of mutant 2′ is even higher than that of AAV9 (1.8-fold for quadriceps (FIG. 2B), 2.6-fold for abdominal muscles (FIG. 4B), and 2.1-fold for hearts (FIG. 3B)).

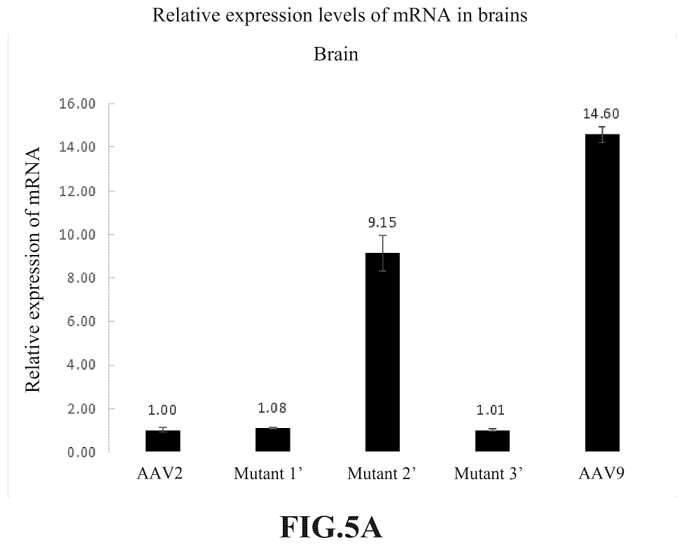

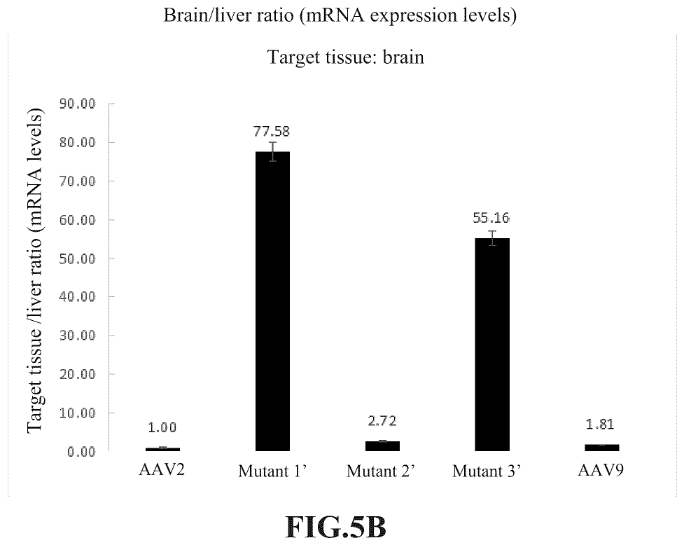

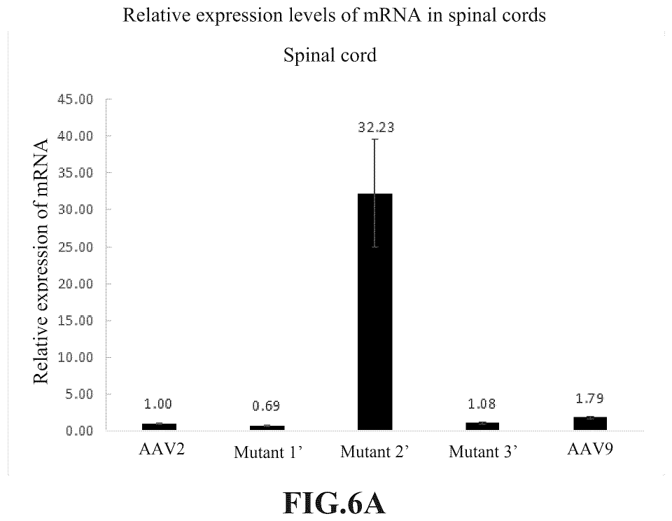

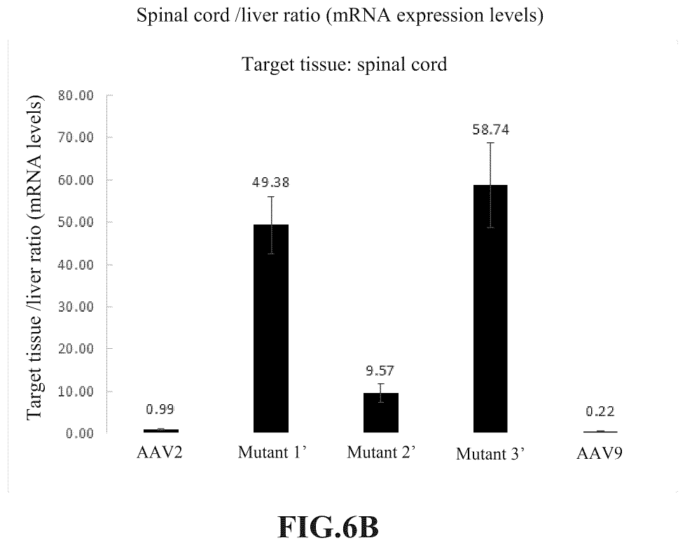

For the targeting to brain and spinal cord, mutant 2′ shows a better targeting advantage over mutants 1′ and 3′. Compared to AAV2, mutant 2′ has a 9-fold and 32-fold higher ability to infect the brain (FIG. 5A) and spinal cord (FIG. 6A), respectively. Mutant 2′ has better infection ability in the spinal cord than AAV9.

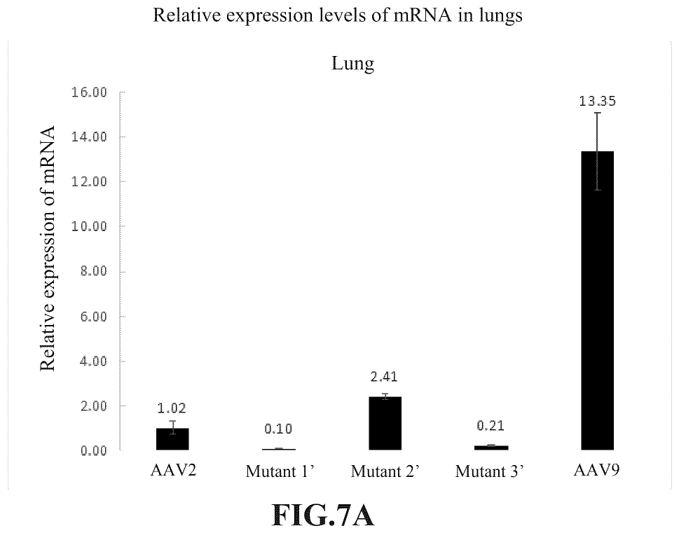

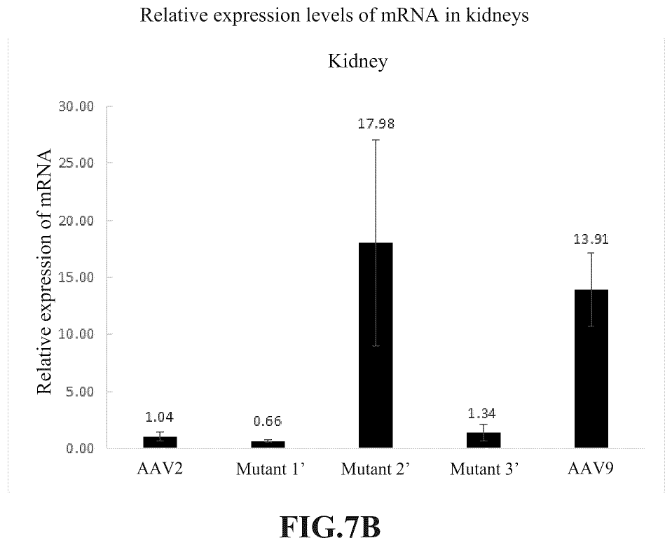

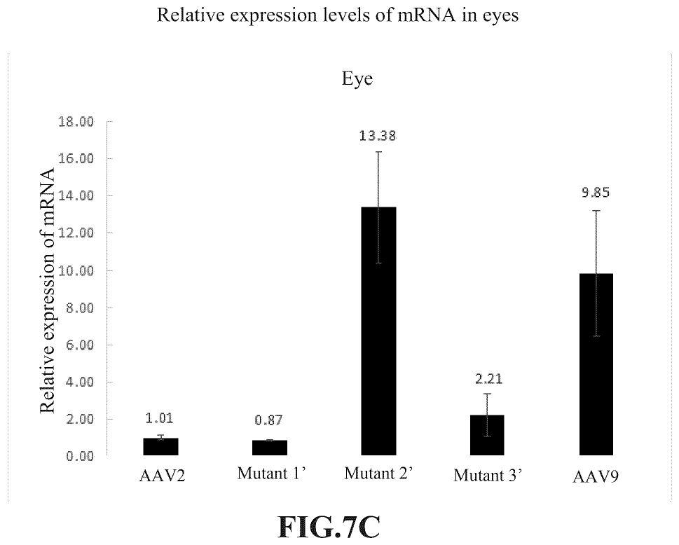

For the targeting to lung, kidney, and eye, mutant 2′ has slightly higher infectivity to the kidney and eye than AAV9, except that the ability to infect the lung is about 5.5 times lower than that of AAV9. Moreover, compared to that of AAV2, infectivity of mutant 2′ was approximately 2.4-fold (FIG. 7A, lung), 18-fold (FIG. 7B, kidney), and 13-fold (FIG. 7C, eye), respectively.

Based on the above results, it is proved that the three mutants with the same backbone sequence have the advantage of low targeting to liver, but shows different characteristics due to the difference of a few amino acids in the ring VIII of CAP protein. For example, the liver tropism of mutant 1′ is 101 times lower than that of AAV2, but mutant 1′ also has low targeting to various organs. Since mutant 1′ is consistent with AAV2 except for the AAV9 sequence replacement at a specific site, it is fully confirmed that the substituted sequence (i.e., the backbone sequence) can greatly reduce the effect of liver tropism, which is also a new discovery of this application. Mutant 2′ includes a further mutation on the base of mutant 1′ (AAV2: R585A), resulting in targeting to muscle and heart comparable to AAV9 and better targeting to spinal cord. In addition to its very low liver tropism, mutant 3′ has a lower ability than AAV9 and a stronger ability than AAV2 in terms of the targeting to muscle and heart, and has low targeting to other organs. Mutant 3′ has a high specific targeting to muscle and heart.

Therefore, in this application, three “chimeras” of AAV serotypes with low liver tropism were constructed through analytical design and “loop swapping” technology, which possesses some advantages of both AAV2 and AAV9. In addition to the characteristics of targeting different organs displayed by the three mutants themselves, based on their excellent target organ/liver ratios (i.e., the ratio of mRNA and/or protein levels in the target organs to those in the liver), they can still be used as a “universal” modified backbone to insert specific sequences such as targeted peptides and antibodies at specific sites to enhance their characteristics of targeting specific tissues but retaining the lower liver tropism, so as to achieve better specificity, avoid the problem of hepatotoxicity in current clinical trials, and provide safer and more reliable gene therapy products for the majority of patients. In this application, the “loop swapping” technique and the rational design analysis method based on AAV structure were used to make use of the complementary advantages of AAV2 and AAV9, with the aim of reconstructing an AAV serotype having low liver tropism and good targeting to specific tissues. In addition, substitution of a sequence segment on the surface of the cap structure (e.g., a protruding ring on the surface) is also a simple and effective way to avoid/resist the influence of a pre-stored neutralizing antibody on the AAV parents (i.e., AAV2 and AAV9) in vivo, and we will further explore it in the future.

### EXAMPLE 3: DESIGN, CONSTRUCTION, AND VIRUS PRODUCTION OF AAV MUTANTS (OR AAV VARIANTS)

### (1) Design of AAV Mutants (Or AAV Variants):

Utilizing the AAV having low liver tropism developed in Examples 1 and 2, we inserted a muscle-targeting RGD peptide at specific sites to form mutant 1 (SEQ ID NO: 27). Additionally, as a relevant control, we also inserted this RGD peptide into the AAV2 backbone variant to form mutant 2 (SEQ ID NO: 28) and mutant 3 (SEQ ID NO: 29), and further performed relevant in vivo activity tests in animals. Subsequently, to extend the tests, we also constructed two other mutants 4 and 5 based on mutant 1 with different RGD peptides inserted (SEQ ID NO: 30, SEQ ID NO: 31).

### (2) Construction of Mutant Serotype Vector and Plasmid Extraction:

The Rep-CAP plasmid was double-digested with SmiI and BshTI, subjected to gel electrophoresis, and a fragment band of about 5000 bp was cut out for gel recovery to obtain a digested backbone fragment.

According to the Cap sequence of mutant 1, the following primers were designed, and the specific steps included: The constructed Rep-CAP plasmid (YJ72) was used as a template and the primers Cap-f+YJ72-R were used for amplification; then the amplified product was recovered by gel extraction to obtain the target product: mutant 1 product-1. The Rep-CAP plasmid of AAV2 was used as a template and primers YJ72-F+YJ107-R were used for amplification; then the amplified product was recovered by gel extraction to obtain the target product: mutant 1 product-2. The Rep-CAP plasmid of AAV2 was used as a template and YJ107-F+cap-r primers were used for amplification; then the amplified product was recovered by gel extraction to obtain the target product: mutant 1 product-3. The Rep-CAP plasmid of mutant 1 can be recombined and constructed by mixing the backbone fragment, mutant 1 product-1,-2, and -3 in the following steps and proportions.

According to the Cap sequence of mutant 2, the following primers were designed, and the specific steps included: The Rep-CAP plasmid of AAV2 was used as a template and primers Cap-f+YJ107-R were used for amplification; then the amplified product was recovered by gel extraction to obtain the target product: mutant 2 product-1. The Rep-CAP plasmid of AAV2 was used as a template and primers YJ107-F+cap-r were used for amplification; then the amplified product was recovered by gel extraction to obtain the target product: mutant 2 product-2. The Rep-CAP plasmid of mutant 2 can be recombined and constructed by mixing the backbone fragment, mutant 2 product-1, and -2 in the following steps and proportions.

According to the Cap sequence of mutant 3, the following primers were designed, and the specific steps included: The Rep-CAP plasmid (YJ69) was used as a template and the primers Cap-f+YJ107-R were used for amplification; then the amplified product was recovered by gel extraction to obtain the target product: mutant 3 product-1. The Rep-CAP plasmid of AAV2 was used as a template and primers YJ107-F+cap-r were used for amplification; then the amplified product was recovered by gel extraction to obtain the target product: mutant 3 product-2. The Rep-CAP plasmid of mutant 3 can be recombined and constructed by mixing the backbone fragment, mutant 3 product-1, and -2 in the following steps and proportions.

According to the Cap sequence of mutant 4, the following primers were designed, and the specific steps included: The mutant 1 plasmid was used as a template and primers Cap-f+249-R were used for amplification; then the amplified product was recovered by gel extraction to obtain the target product: mutant 4 product-1. The mutant 1 plasmid was used as a template and primers 249-F+cap-r were used for amplification; then the amplified product was recovered by gel extraction to obtain the target product: mutant 4 product-2. The Rep-CAP plasmid of mutant 4 can be recombined and constructed by mixing the backbone fragment, mutant 4 product-1, and −2 in the following steps and proportions.

According to the Cap sequence of mutant 5, the following primers were designed, and the specific steps included: The mutant 1 plasmid was used as a template and primers Cap-f+250-R were used for amplification; then the amplified product was recovered by gel extraction to obtain the target product: mutant 5 product-1. The mutant 1 plasmid was used as a template and primers 250-F+cap-r were used for amplification; then the amplified product was recovered by gel extraction to obtain the target product: mutant 5 product-2. The Rep-CAP plasmid of mutant 5 can be recombined and constructed by mixing the backbone fragment, mutant 5 product-1, and -2 in the following steps and proportions.

The primers involved in the construction of the Rep-CAP vector of mutants 1-5 of the AAV capsid protein in this application are:

| Primer name | Primer sequence (5′ → 3′) |
|---|---|
| Cap-f | CATCTTTGAACAATAAATGATTTAAATCAGGTATG (SEQ ID NO: 13) |
| cap-r | TCAACTGAAACGAATCAACCGGTTT (SEQ ID NO: 14) |
| YJ107-R | GAGTTGTAGTCTCCTCTGCTGTTGCTCTGGAGGTTGGTAGATACAGAACCATACT |
| | (SEQ ID NO: 40) |
| YJ107-F | GCAACAGCAGAGGAGACTACAACTCCCTGAGACAAGCAGCTACCGCAGAT (SEQ ID |
| | NO: 41) |
| YJ72-R | GTTGTTTTGAGTCACAGTGGTTGATACTCGCTGCTGGCGGTAACAG (SEQ ID NO: 17) |
| YJ72-F | ATCAACCACTGTGACTCAAAACAACAACAGTGAATACTCGTGGACTGGAG (SEQ ID |
| | NO: 18) |
| 249-R | TGTTGTCTGGTCTCCTCTTCCAGGTCCCTGGAGGTTGGTAGATACAGAACCATACT |
| | (SEQ ID NO: 42) |
| 249-F | CCTGGAAGAGGAGACCAGACAACACTGAGACAAGCAGCTACCGCAGAT (SEQ ID |
| | NO: 43) |
| 250-R | ATTGTTGAAATCGCCTCGTCTATTCTCCTGGAGGTTGGTAGATACAGAACCATACT |
| | (SEQ ID NO: 44) |
| 250-F | AATAGACGAGGCGATTTCAACAATACCAGACAAGCAGCTACCGCAGAT (SEQ ID NO: |
| | 45) |

There are homology arm sequences between the backbone fragment and the fragment and between the fragments, and multiple fragments can be assembled into a complete vector through Gisbon. One clean PCR tube with a volume of 200 μL was taken, labeled, and placed on an ice box. The reaction solution was prepared with the digested backbone fragment above and each target fragment (i.e., the target product above). In the reaction solution, the molar ratio of the digested backbone fragment to each target fragment was 1:3. The reaction solution was reacted in a PCR instrument at 50° C. for 30 min to perform recombination ligation. 50 μL of competent cells were thawed on ice, and 10 μL of ligation product was mixed with DH5a competent cells. The resulting mixture was placed on ice for 20-30 min; heat-shocked at 42° C. for 45 seconds; quickly placed in an ice bath on ice for 2 min, and then added to 400 μL of SOC medium (without antibiotics) for recovery and incubated at 37° C., 200 rpm for 1 h. The incubated product was evenly spread on an Amp-resistant plate (50 μg/ml) and cultured at 37° C. for 14 hours. The monoclonal bacteria were selected and expanded in 4 mL of liquid LB medium (Amp+ resistance) for 14 hours at 37° C.

The bacterial solution was centrifuged at 12,000 rpm for 1 min and the supernatant medium was poured off to obtain an EP tube containing the bacterial precipitate. 250 μL of a mixture of buffer P1/RNaseA was added to the EP tube and the bacteria were resuspended through vortex at a high speed. Subsequently, 250 μL of buffer P2 was added to the EP tube and then the EP tube was turned up and down 8-10 times. Then, 350 μL of buffer P3 was added to the EP tube, and the solution was immediately inverted and mixed 8-10 times to allow for thorough neutralization. The EP tube was centrifuged at 13,000 rpm for 10 minutes, and the resulting supernatant was passed through the column. The centrifuge tube containing the resulting column was centrifuged at 12,000 rpm for 1 min and the waste liquid was discarded. 500 μL of PW1 was added to the centrifuge tube, the resulting centrifuge tube was centrifuged at 12,000 rpm for 1 min, and the waste liquid was discarded. 600 μL of PW2 was added to the centrifuge tube, the obtained centrifuge tube was centrifuged at 12,000 rpm for 1 min, and the supernatant was poured off. 600 μL of PW2 was added to the centrifuge tube, the resulting centrifuge tube was centrifuged at 12,000 rpm for 1 min, and the supernatant was poured off. After the centrifuge tube was centrifuged without load at 12,000 rpm for 2 min, 30-50 μL of eluent pre-heated at 55° C. was added, the centrifuge tube was allowed to stand for 2 min, and centrifuged at 12,000 rpm for 1 min. Concentration detection was performed using a micro nucleic acid quantifier.

The obtained plasmid was tested for concentration, and 10 μL of the positive plasmid identified by enzyme digestion was taken for sequencing, and the positive plasmid was stored at −20° C. The sequencing results show that the obtained plasmid could encode a mutant capsid protein VP1. Finally, according to the amount of virus required for later testing, the relevant helper plasmids, each group of Rep-Cap plasmids (AAV2, AAV9, and mutants in this application) and GOI plasmids (ssAAV.CAG.Fluc-2a-eGFP.WPRE.SV40pA) were extracted.

### (3) Packaging and Purification of Mutants of Virus Serotypes

The Rep-Cap plasmids of each group (AAV2, AAV9, MyoAAV 4A, MyoAAV 2A, MyoAAV 4E, and the mutants of AAV of the present application), a plasmid expressing firefly luciferase (Fluc) and green fluorescent protein (EGFP), and a pHelper plasmid were co-transfected into HEK-293T cells in an appropriate amount, and then the AAV viruses were purified by iodixanol gradient ultracentrifugation. The measured virus titer was 1E+12GC/mL-1E+13GC/mL, which was a suitable titer. The obtained AAV viruses were stored at −80° C. for future use. For MyoAAV 4A, MyoAAV 2A, and MyoAAV 4E, see descriptions in Tabebordbar M et al., Directed evolution of a family of AAV capsid variants enabling potent muscle-directed gene delivery across species. Cell. 2021 Sep. 16; 184 (19): 4919-4938.

### EXAMPLE 4: COMPARATIVE TEST OF VARIOUS INDICAORS OF MUTANT SEROTYPES

### (1) Animal Injection and Dissection

Mouse experiments: Experiments were performed using 6-8 week old C57 male mice. The relevant viruses were formulated according to the designed experimental and control groups. Each group was injected with 2E11GC viruses per mouse. Animal dissection and sampling of various organs were performed 4 weeks after injection. Samples were immediately frozen in liquid nitrogen after sampling and used for subsequent experiments such as RNA extraction and western blot (WB) detection, respectively.

Cynomolgus monkey (Macaca fascicularis) experiments: Experiments were conducted using 4-5 year old male cynomolgus monkeys. Serum was taken before injection for a serotype-specific neutralizing antibody, after meeting the standard, the relevant viruses were prepared according to the designed experimental group and control group. Each monkey was injected with virus at 3E13 GC/Kg. Animals were observed daily to record animal status and blood was collected at different time points for relevant biochemical tests (fully automatic biochemistry analyzer of Roche Cobas C311). Samples were collected by puncture 14 and 28 days after injection (5 points in total were taken from each monkey: liver (only 14 days after injection), gastrocnemius, quadriceps, biceps, and triceps).

### (2) Detection of DNA/mRNA Expression Levels of Target Genes

### (2.1) Extraction of Tissue DNA

Sample grinding: The grinder was pre-cooled 10 min in advance and the grinding parameters were set. An animal tissue sample stored in a −80° C. refrigerator was taken out. About 1-10 mg of the tissue was taken from the sample, clipped to the size of soybean grains in a sterile petri dish, then transferred to a 1.5 mL RNase-free EP tube. Then, two clean and sterile steel grinding balls with a diameter of 3 mm were added into the EP tube and the EP tube was wrapped with a sealing film. The sample was placed and leveled in a 24-well grinding adapter, the screw was tightened, and the cap-off button was pressed. The grinding program was initiated, after the program was finished, the sample was taken out and the particle size of the sample after grinding was observed; if no large piece of the tissue remained, the subsequent extraction operation could be carried out. After grinding, the sample was centrifuged at 12,000×g and 4° C. for 2 min, and the supernatant was transferred to a new RNase-free 1.5-mL eppendorf (EP) tube labeled accordingly.

Extraction of total DNA from a sample: The specific operation was carried out according to the instruction manual of HiPure Universal DNA Kit (Guangzhou Magen Biotechnology Co., Ltd., Item No.: D3018-03). 200 μl of ATL and 20 μl of proteinase K were added to each EP tube containing a tissue solution obtained by grinding, which was then bathed at 55° C. for 30-60 min. 10 μl of RNase A was added to the EP tube, which was then allowed to stand at room temperature for 10 min. 200 μl of AL was added to the resulting EP tube, which was then vortexed at high speed for 10 s, followed by a warm bath at 70° C. for 10 min. 200 μl of anhydrous ethanol was added to the obtained EP tube, which was then vortexed at high speed for 10 s. The HiPure DNA Mini Column I was loaded into a 2 ml collection tube and the resulting mixture (including precipitate) was transferred to the column, which was then centrifuged at 10,000×g for 1 min at room temperature. The resulting column was loaded into a new collection tube and 500 μl of GW 1 (diluted with ethanol) was added to the column, which was then centrifuged at 10,000×g for 1 min at room temperature. A first waste solution from centrifugation was discarded, the column was loaded back into the collection tube, and 650 μl of GW2 (diluted with ethanol) was added to the column, which was then centrifuged at 10,000×g for 1 min at room temperature. A second waste liquid from centrifugation was discarded and the resulting column was loaded back into the collection tube, which was then centrifuged at 10,000×g for 3 min at room temperature. The obtained spin column was placed into a new 1.5 ml EP tube and 20-200 μl of sterile water preheated to 70° C. was added to the membrane center of the spin column. The resulting spin column was allowed to stand at room temperature for 3 min, and then centrifuged at 10,000×g for 1 min at room temperature. The DNA binding column was discarded and the resulting DNA was stored temporarily at 2-8° C.

Determination of nucleic acid concentration in the sample: The DNA concentration was detected using a micro nucleic acid quantifier, the concentration, OD260/280, and OD260/230 were recorded, and the DNA was stored at −80° C.

### (2.2) Total RNA Extraction From a Tissue and Reverse Transcription:

Sample grinding: The grinder was pre-cooled 10 min in advance and the grinding parameters were set. An animal tissue sample stored in a −80° C. refrigerator was taken out. About 50-100 mg of the tissue was taken from the sample, clipped to the size of soybean grains in a sterile petri dish, then transferred to a 1.5 mL RNase-free EP tube. At the ratio of 1 mL of TransZol Up per 50-100 mg of the tissue, an appropriate amount of TransZol Up was added to the EP tube, two clean and sterile steel grinding balls with a diameter of 3 mm were then added, and the tube was sealed with parafilm. The sample was placed and leveled in a 24-well grinding adapter, the screw was tightened, and the cap-off button was pressed. The grinding program was initiated, after the program was finished, the sample was taken out and the particle size of the sample after grinding was observed; if no large piece of the tissue remained, the subsequent extraction operation could be carried out. After grinding, the sample was centrifuged at 12,000×g and 4° C. for 2 min, and the supernatant was transferred to a new RNase-free 1.5-mL eppendorf (EP) tube labeled accordingly.

Extraction of total RNA from a sample: For details, refer to the instruction manual of TransZol Up Plus RNA Kit (Beijing TransGen Biotech Co., Ltd., Item No.: ER501). Whenever 1 ml of TranZol Up was used, 0.2 mL of the RNA extraction agent was added to the sample and the resulting mixture was shaken vigorously for 5 min; centrifugation was performed at 12,000×g and 4° C. for 10 min. At this point, the sample was divided into three layers, and the colorless aqueous phase was transferred to a new RNase-free 1.5-mL EP tube, to which an equal volume of anhydrous ethanol was added (precipitation may occur at this point), and the EP tube was gently inverted to mix evenly. The obtained solution together with the precipitate was added to the spin column, and the spin column was centrifuged at 12,000×g for 30 seconds at room temperature, and the filtrate was discarded. 500 μL of CB9 was added to the spin column, which was then centrifuged at 12,000×g for 30 seconds at room temperature, and the filtrate was discarded; the previous step was repeated once. 500 μL of WB9 was added to the spin column, which was then centrifuged at 12,000×g for 30 seconds at room temperature, and the filtrate was discarded; the previous step was repeated once. The spin column was centrifuged at 12,000×g for 2 min at room temperature to completely remove residual ethanol. The spin column was placed into a RNase-free 1.5-mL EP tube and 30-50 μL (depending on tissue size) of RNase-free water was added to the center of the spin column and allowed to stand for 1 min at room temperature. The EP tube containing the spin column was centrifuged at 12,000×g for 1 min at room temperature to elute RNA.

Determination of nucleic acid concentration in the sample: The RNA concentration was detected using the micro nucleic acid quantifier, the concentration, OD260/280, and OD260/230 were recorded, and the RNA was stored at −80° C.

Reverse transcription: EasyScript® All-in-One First-Strand cDNA Synthesis SuperMix for qPCR (One-Step gDNA Removal) (Beijing TransGen Biotech Co., Ltd., Item No.: AE341-03) was used for each group of RNA samples, and the specific steps were described in the instruction manual.

(2.3) Quantitative PCR (qPCR) Experiments:

Each group of DNA or cDNA was used as a template and the qPCR system was prepared according to the instructions of 2× SYBR Green qPCR Master Mix (BIMAKE, Item No.: B21203):

### qPCR System

| Reagent | Usage | |
|---|---|---|
| 2× SYBR Green qPCR Master Mix | 10 | μL |
| Template (DNA or cDNA) | 1.5 | μL |
| Upstream primer (10 μM) | 1 | μL |
| Downstream primer (10 μM) | 1 | μL |
| ROX Reference Dye | 0.4 | μL |

| | |
|---|---|
| Deionized water | Up to 20 μL |

| Primer name | Primer sequence (5′ → 3′) |
|---|---|
| Fluc2-qPCR-F1 | AACCAGCGCCATTCTGATCA (SEQ ID NO: 23) |
| Fluc2-qPCR-R1 | TCGGGGTTGTTAACGTAGCC (SEQ ID NO: 24) |
| GAPDH-qPCR-F | GTCTCCTCTGACTTCAACAGCG (SEQ ID |
| | NO: 46) |
| GAPDH-qPCR-R | ACCACCCTGTTGCTGTAGCCAA (SEQ ID |
| | NO: 47) |

### Settings of qPCR Procedure

| | 1 | | | | | | | | | | | |
|---|---|---|---|---|---|---|---|---|---|---|---|---|
| | Hot-Start | | | | | | | | | | | |
| | DNA | | 2 | | | | | | | | | |
| | Polymerase | | PCR | | | | 3 | | | | | |
| | Activation | | Cycle (40 cycles) | | | | Melt Curve | | | | | |
| Step | Hold | | Denature | | Anneal/Extend | | Cycle (1 cycle) | | | | | |
| Temp | 95° | C. | 95.0° | C. | 60.0° | C. | 95.0° | C. | 60.0° | C. | 95° | C. |
| Time | 10 | min | 15 | sec | 30 | sec | 15 | sec | 60 | sec | 15 | sec |

### (2.4) Data Analysis

Relative expression was calculated from the Ct values of each group according to the formula 2\hat{ }-ΔΔct.

### (3) WB Detection for Expression Levels of Target Proteins

Sample pre-treatment: The tissue was cut into tiny pieces and weighed. The weight of the tissue was recorded. The tiny pieces were placed in a 1.5 mL or 2 mL centrifuge tube; and the centrifuge tube was labeled and frozen at −80° C. for use. The cryo-grinder was pre-chilled. RIPA (Shanghai Beyotime Biotechnology Co., Ltd., P0013B) lysate was solubilized (Phenylmethylsulfonyl fluoride (PMSF) was added within a few minutes prior to use to obtain a final PMSF concentration of 1 mM).

The above complete lysate was added to the centrifuge tube at a ratio of 150-250 μL of lysate per 20 mg of the tissue. Then two sterilized zirconium oxide grinding balls were added to the centrifuge tube and the sample was ground directly in the lysis buffer (the tissue sample of the brain or spinal cord: The temperature was −20° C., and the frequency was 70 Hz; the oscillation time was 50 seconds followed by a pause of 10 seconds, and this cycle was repeated 3-4 times; and the sample of the muscle or liver: The temperature was −20° C., and the frequency was 70 Hz; the oscillation time was 50 seconds followed by a pause of 10 seconds, and this cycle was repeated 5-7 times). After grinding, the sample was centrifuged in a freezing centrifuge at 4° C., 12,000×g for 5-10 min, and then the supernatant was transferred to a new sterilized EP tube and stored at −20° C. or −80° C.

Determination of protein concentration: Protein concentration was determined according to the method in the protein concentration assay kit based on the modified BCA method (Sangon Biotech (Shanghai) Co., Ltd., Item No.: C503051). Then, an appropriate amount of a sample of homogenate protein was taken according to the required amount, mixed with the corresponding amount of 5× SDS-PAGE protein loading buffer, boiled in a water bath for 10 min, cooled and centrifuged at a low speed for a while, and waited for sample loading.

Western blot (WB) detection:

A. SDS-PAGE electrophoresis: The appropriate loading amount was determined by the protein concentration and expression level, and was less than 20 μL/well. The loading amount of the tissue homogenate protein was about 20-50 μg. The specific operation process of electrophoresis was as follows: The comb on the precast gel was pulled out and the gel was mounted in the electrophoresis chamber. The electrophoresis buffer was added to the inner and outer chambers, where the inner chamber was filled with a freshly prepared buffer and checked for leaks. If there was no leakage in the inner chamber, then electrophoresis buffer could be added to the outer chamber. An appropriate amount of a treated protein sample was taken for loading, the pre-stained standard protein was used as a reference, and electrophoresis was carried out on a Tanon (Shanghai tanon life science Co., Ltd.) electrophoresis device at a constant voltage of 100 V for 100 min until the bromophenol blue reached the bottom of the gel. The power was turned off, the precast gel plate was carefully unloaded, and the gel was removed and placed in the transfer buffer to await subsequent manipulation.
B. Transfer: Six sheets of filter paper and one sheet of PVDF membrane were cut according to the gel area. The PVDF membrane was soaked in methanol for 5-10 seconds and then transferred to and soaked in the transfer buffer for 5 min, and the filter paper was also pre-wetted in the transfer buffer. Installation of the transfer device: Negative electrode (black plate)—sponge—3 layers of moistened filter paper—gel—PVDF membrane—3 layers of moistened filter paper—sponge—positive electrode (transparent plate). Each layer of air bubbles in the transfer device was driven away to avoid affecting the transfer effect, and after clamping the bracket, the transfer device was placed into the electrotransfer tank. The membrane was transferred in an ice bath at a constant voltage of 100 V for 100 min. The success of membrane transfer was determined by whether protein bands of the pre-stained molecular weight marker were completely transferred to the PVDF membrane. The transferred PVDF membrane was soaked and washed in PBST solution at room temperature for 5 min, and the PVDF membrane was cut according to the requirements. Be careful not to let the PVDF membrane dry out during the cutting process.
C. Blocking and antibody incubation: The PVDF membrane was incubated with a blocking solution (5% skimmed milk powder) for 2 hours at room temperature or overnight at 4° C. The blocked PVDF membrane was transferred to a primary antibody hybridization solution and incubated for 1 h at room temperature or overnight at 4° C. Luciferase Rabbit Polyclonal antibody (Proteintech Group, Inc., 27986-1-AP) was added to 4 mL of QuickBlock™ Western primary antibody dilution (Shanghai beyotime biotechnology Co., Ltd., P0256) in a ratio of 1:2000 to prepare the primary antibody hybridization; GADPH Rabbit Polyclonal antibody (Proteintech Group, Inc., 10494-1-AP) was added to 4 mL of QuickBlock™ Western primary antibody dilution (Shanghai beyotime biotechnology Co., Ltd., P0256) in a ratio of 1:2000 to prepare the primary antibody hybridization; and Rabbit GFP tag Polyclonal antibody (Proteintech Group, Inc., 50430-2-AP) was added to 4 mL of QuickBlock™ Western primary antibody dilution (Shanghai beyotime biotechnology Co., Ltd., P0256) in a ratio of 1:2000 to prepare the primary antibody hybridization. Then the membrane was washed three times with PBST for 5 min each time. The washed PVDF membrane was transferred into the secondary antibody hybridization solution and incubated at room temperature for 1 h. HRP-conjugated Affinipure Goat Anti-Rabbit IgG (H+L) (Proteintech Group, Inc., SA00001-2) was added to 4 mL of QuickBlock™ Western Secondary Antibody Diluent (Shanghai beyotime biotechnology Co., Ltd., P0258) at a ratio of 1:5000 to obtain the secondary antibody hybridization solution. The membrane was washed three times with PBST for 3×5 min each time.
D. Color development: ECL chemiluminescence kit's liquid A and liquid B were mixed in an equal volume. After shaking and mixing, the luminescent liquid was dropped on the PVDF membrane such that all the PVDF membrane was covered with the luminescent liquid, and the exposure time was adjusted such that the protein bands were clear, and then photographed by the instrument.

A previously developed novel AAV vector with low liver tropism was utilized and envisioned to have better specificity and retain low hepatotoxicity by inserting different targeting peptides. By inserting different RGD peptides with muscle targeting into the backbone, and designing different wild-type control groups (AAV2, AAV9) and corresponding groups including a polypeptide insertion, the present application validated the AAV mutants (or AAV variants) by in vivo analyses in mice and cynomolgus monkeys.

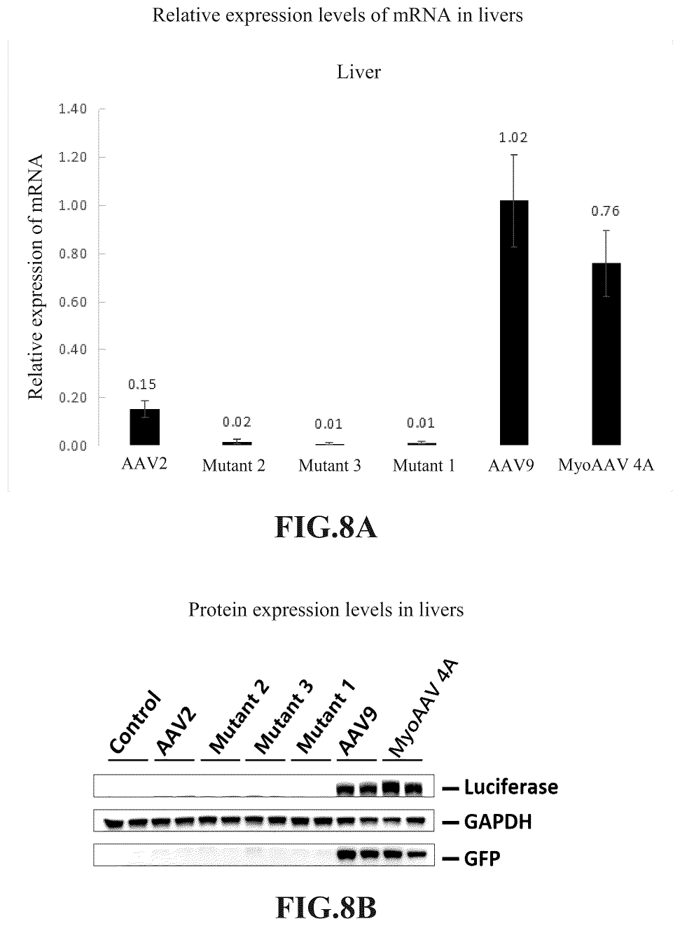

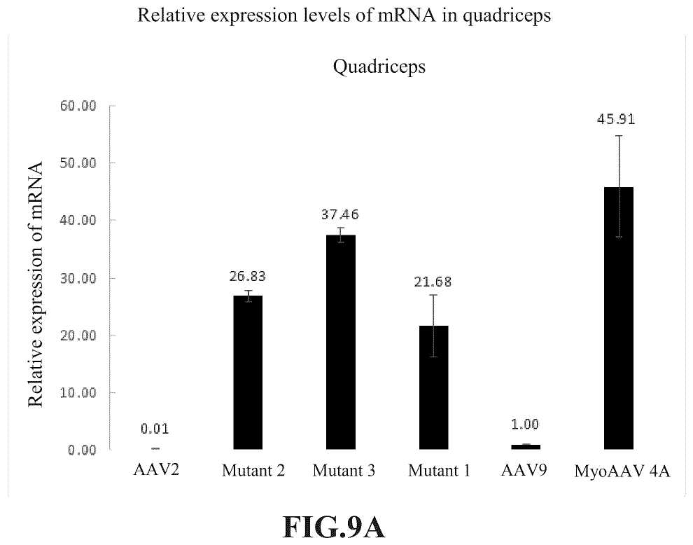

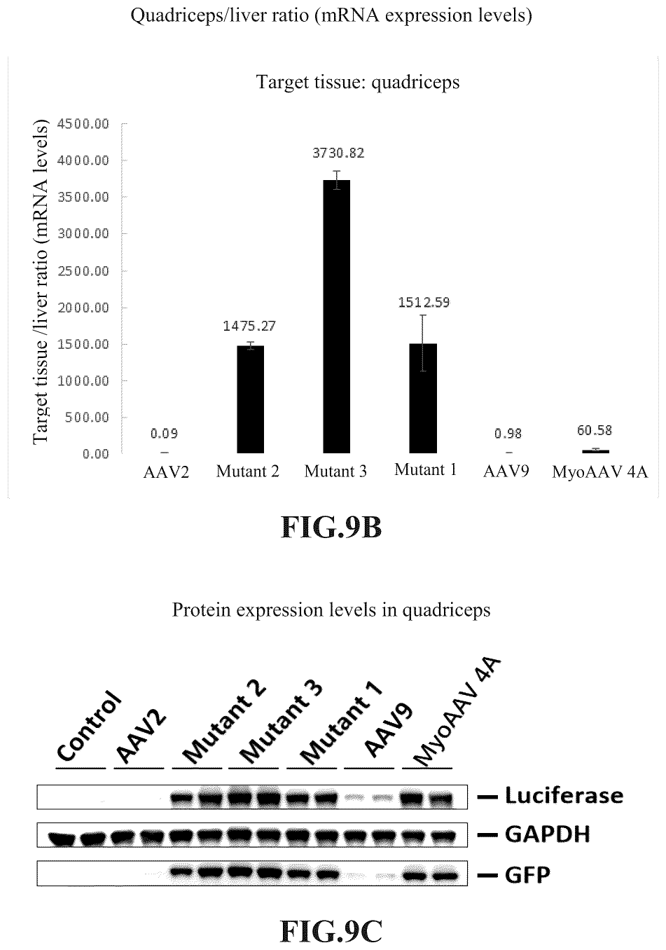

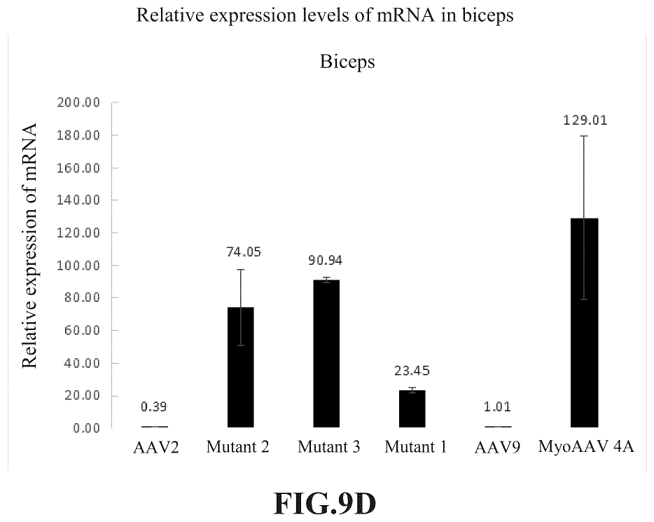

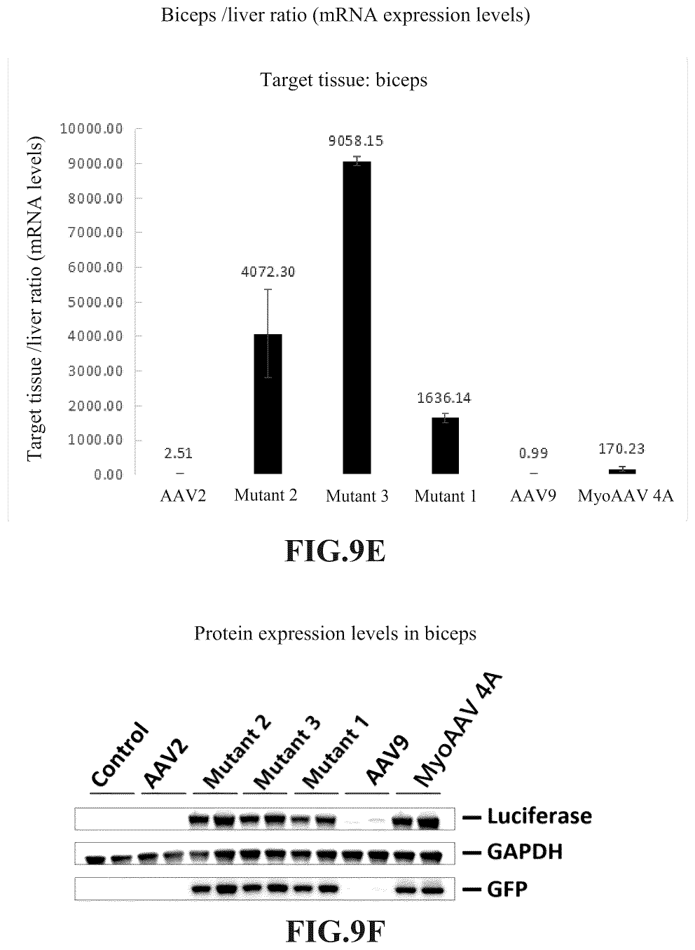

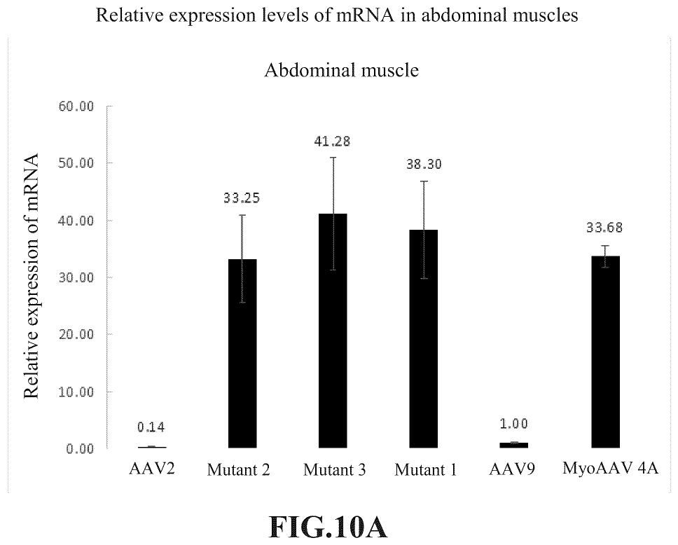

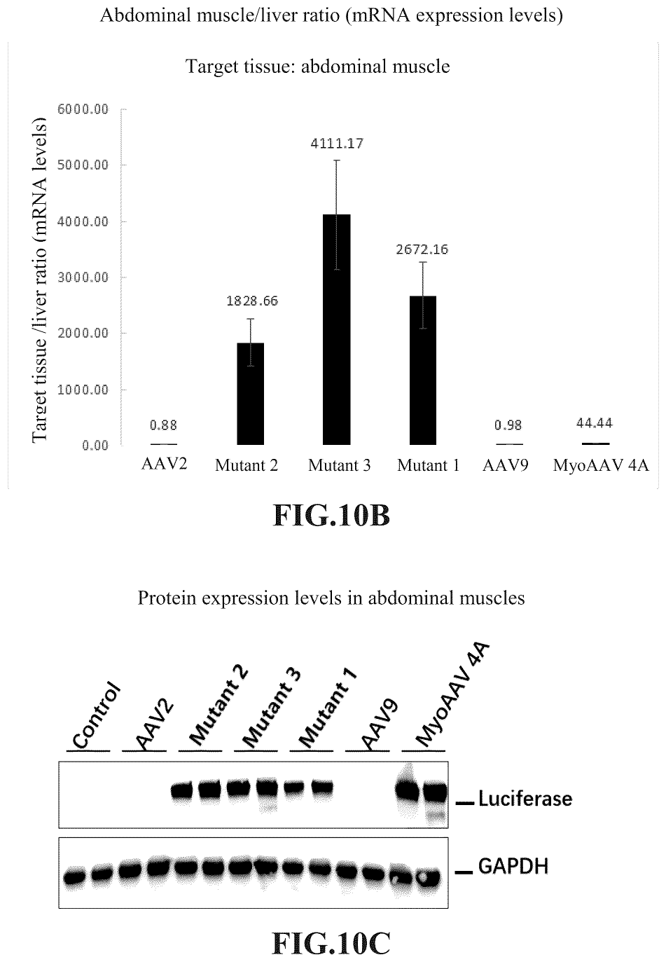

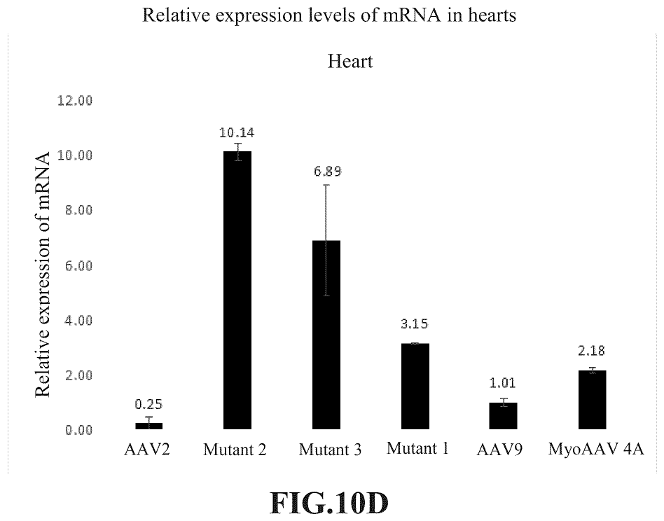

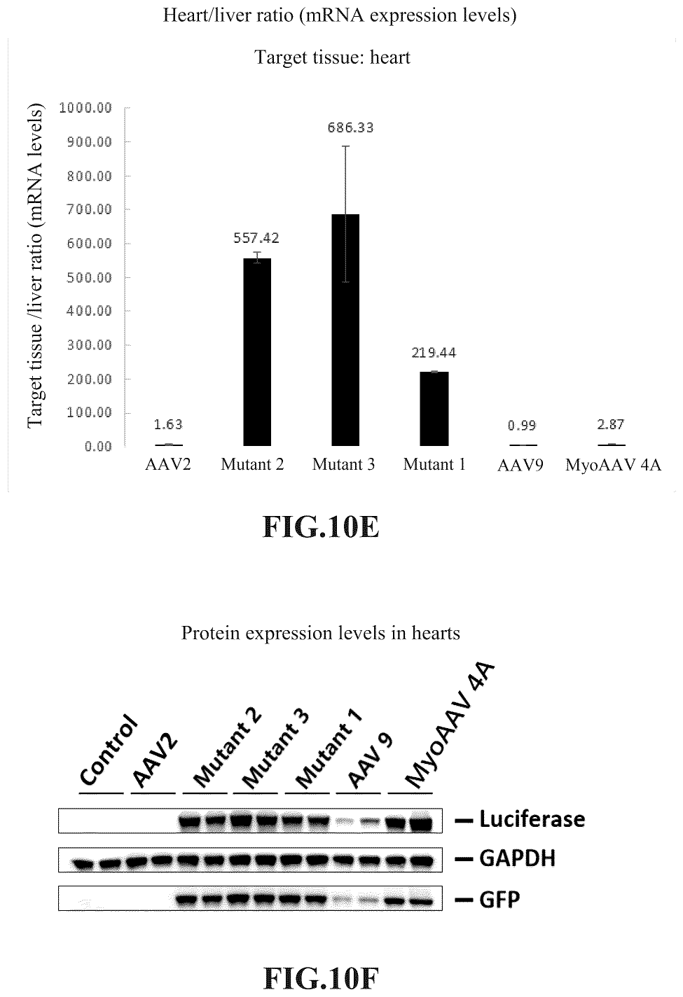

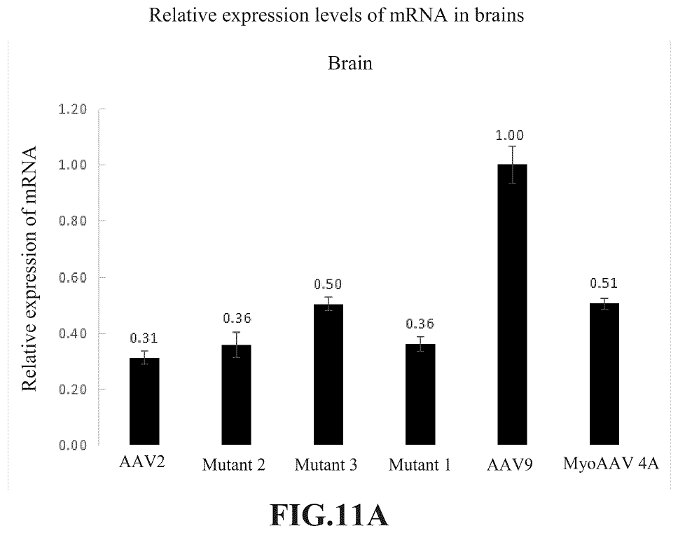

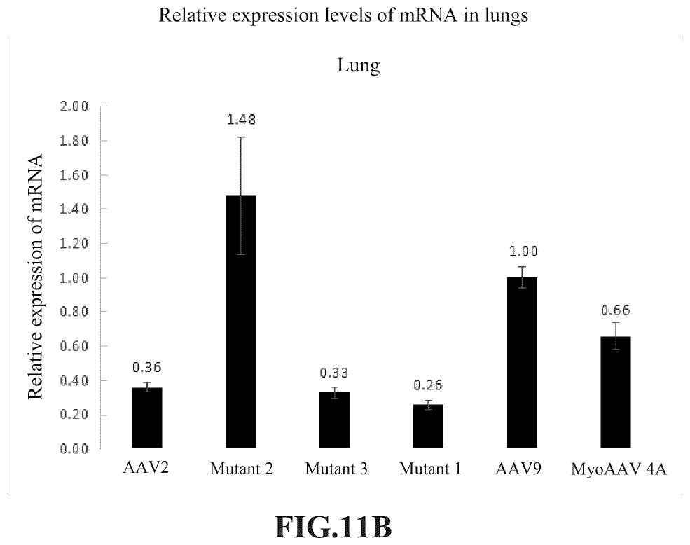

In mouse experiments, mutants 1, 2, and 3 all have the characteristic of low liver tropism. Both mutants 1 and 3 have the same low liver tropism, which is 102-fold lower than that of AAV9, 76-fold lower than that of MyoAAV 4A, and 15-fold lower than that of AAV2 (FIG. 8A), which is consistent with the results at the protein levels (FIG. 8B). Regarding muscle targeting, mutants 1, 2, and 3 into which the RGD peptide is inserted all retain the muscle targeting to a certain extent. For example, the mRNA expression level of mutant 3 in quadriceps is 37-fold (90-fold for biceps, and 38-fold for abdominal muscles) that of AAV9 (FIGS. 9A and 9D; FIG. 10A), and the mRNA expression level of mutant 3 in hearts is 6-fold that of AAV9 (FIG. 10D). Compared with the MyoAAV 4A serotype with strong muscle targeting, the mRNA expression levels of mutant 3 in quadriceps and biceps are slightly lower (0.82-fold, 0.7-fold), and slightly higher in abdominal muscles and hearts (1.2-fold, 3.2-fold). Among them, the mRNA levels in the quadriceps and biceps of mutant 1 are significantly different from those of MyoAAV 4A (0.47-fold and 0.18-fold), but the mRNA levels in the abdominal muscles and hearts of mutant 1 are slightly higher. The protein expression levels of mutants are basically consistent with the mRNA expression levels, but the difference in protein expression is not large (Regarding the quadriceps in FIG. 9C, the biceps in FIG. 9F, the abdominal muscles in FIG. 10C, and the hearts in FIG. 10F, the protein expression levels of mutants 1, 2, and 3 in the above tissues are much higher than those of AAV9, and even reach levels similar to those of MyoAAV 4A). At the same time, we analyzed the targeting of mutants to other organs, such as the brain. The infection ability of mutants 1, 2, 3 and MyoAAV 4A to the brain each was weaker than that of AAV9 (FIG. 11A). Mutant 2 has a slightly higher infection ability to the lung than AAV9 (FIG. 11B). It can be seen that RGD peptide insertion has successfully improved muscle targeting of mutants 1, 2, and 3 (but our previous studies have found that not every serotype can obtain this improved property by direct insertion, such as AAV6), and mutants 1, 2, and 3 can show muscle targeting comparable to MyoAAV 4A. However, due to sequence differences, the degree of improvement in this function varies among different mutants. The advantages of mutants 1, 2 and 3 of the present application are that they exhibit greatly reduced liver tropism while retaining their ability of muscle targeting. For example, mutant 3 performs best in mouse muscles, with muscle/liver ratios of 61.6, 53.2, and 92.5 times those of MyoAAV 4A (quadriceps, biceps, and abdominal muscles, corresponding to FIGS. 9B and 9E; FIG. 10B), and a heart/liver ratio of 238 times that of MyoAAV 4A (FIG. 10E).

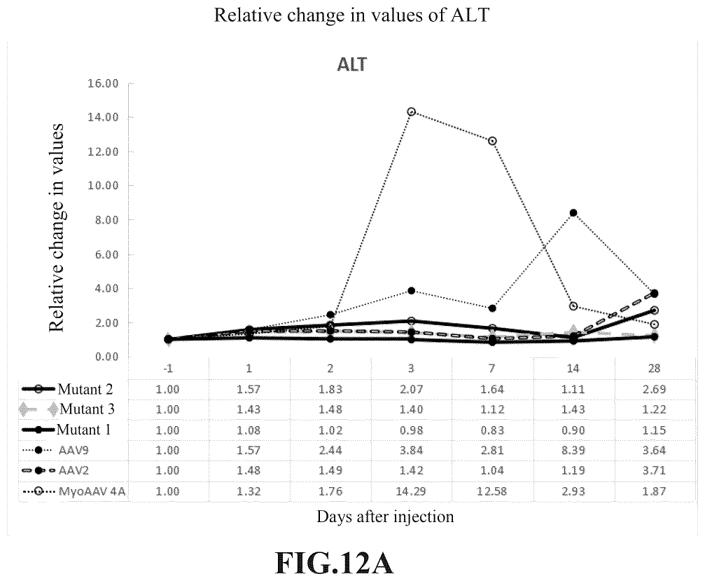

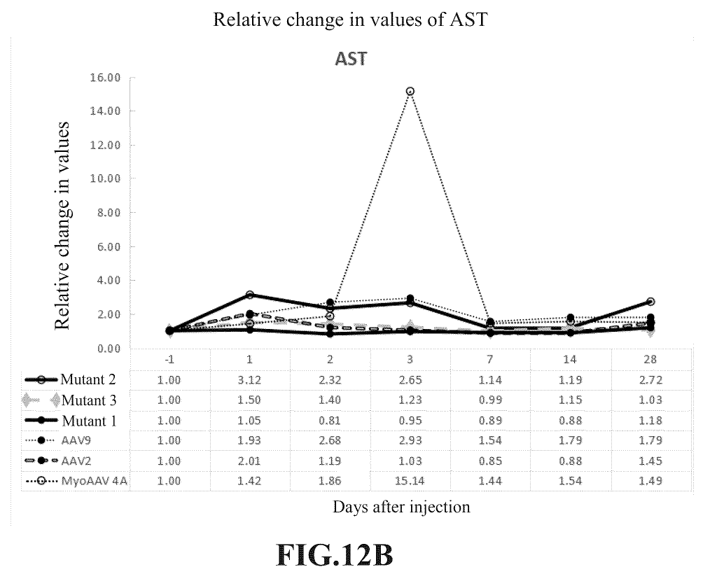

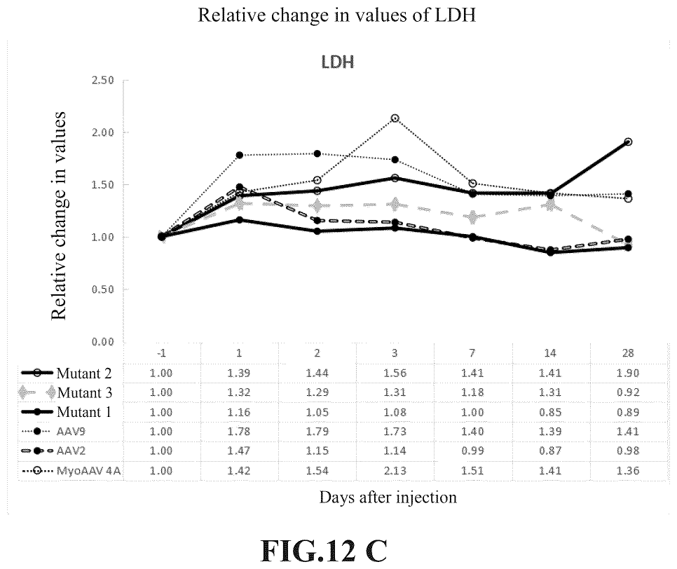

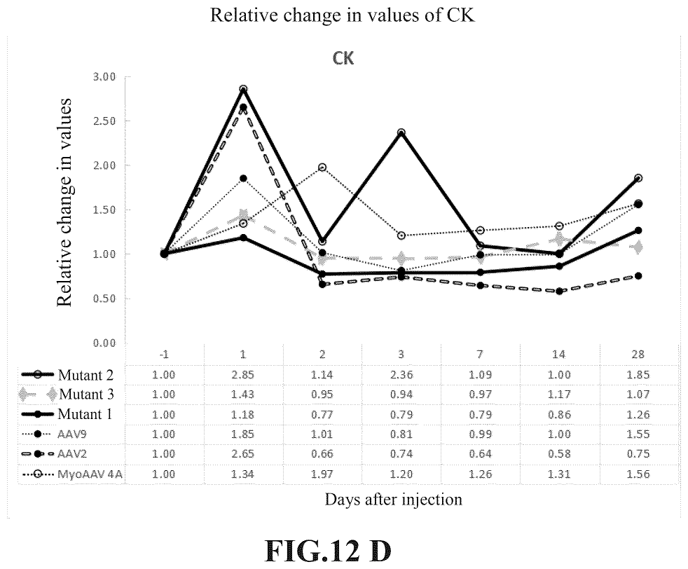

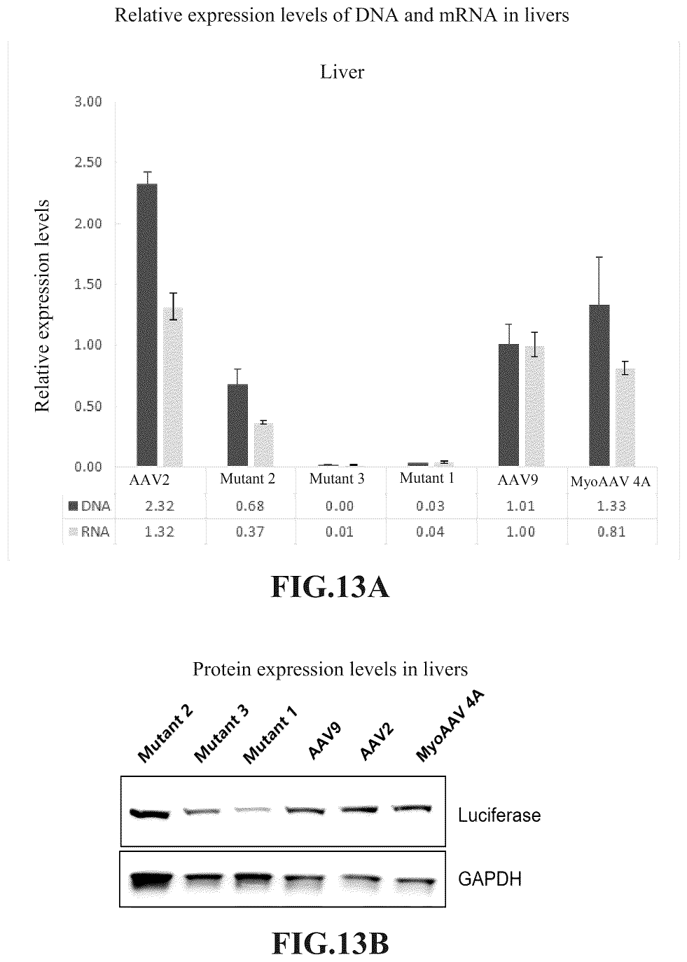

In order to better apply the serotypes to clinical research, we have further evaluated the effects of the mutants of the present application in cynomolgus monkeys (i.e.,Macaca fascicularis). Similarly, we have investigated the liver tropism and toxicity of AAV serotypes, as well as their effects in muscles of different locations. Through the analysis of biochemical indicators at different time points (FIGS. 12A-12D), we find that the ALT and AST levels of MyoAAV 4A (two main indicators used clinically to evaluate liver injury) soars to the observed peak on the third day after injection, reaching 14.29 times and 15.14 times the levels before injection, respectively. The second largest changes of the ALT and AST levels are observed in cynomolgus monkeys injected with AAV9, with the observed peak reaching 8.39 times the levels before injection on the 14th day. Minimal fluctuations in ALT and AST levels are observed in the cynomolgus monkeys injected with mutants 1, 2, and 3, with mutant 1 being optimal in particular, showing very stable performance in both of the two indicators. Mutant 1 and mutant 3 exhibit the lowest levels of hepatic DNA, mRNA, and protein expression (FIGS. 13A and 13B) (for example, the mRNA levels of mutant 1 and mutant 3 are 20.25-fold and 81-fold lower than that of MyoAAV 4A, respectively), as well as minimal changes in ALT and AST. The increase levels of LDH (lactate dehydrogenase) of both AAV9 and MyoAAV 4A (a mutant obtained by inserting a RGD peptide into AAV9) are also greater than those of other serotypes, while the changes in CK (creatine kinase) levels of both AAV2 2and mutant 2 (a mutant obtained by inserting a RGD peptide into AAV2) are greater, which may be related to the characteristics of the serotypes.

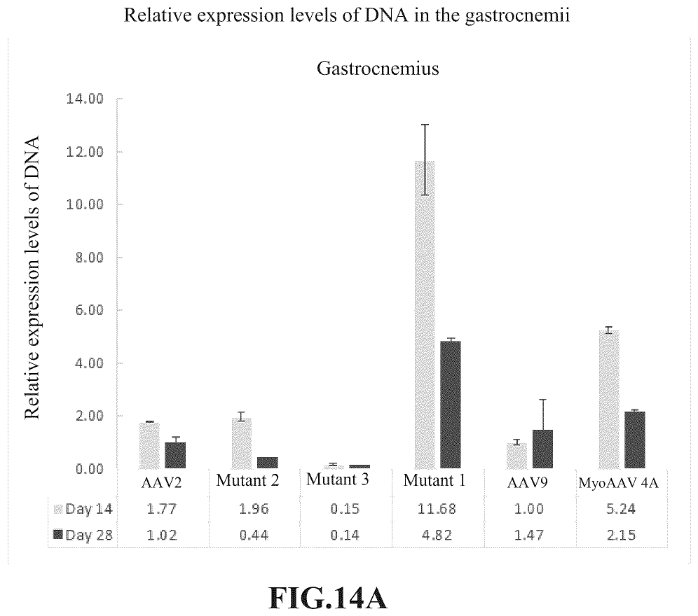

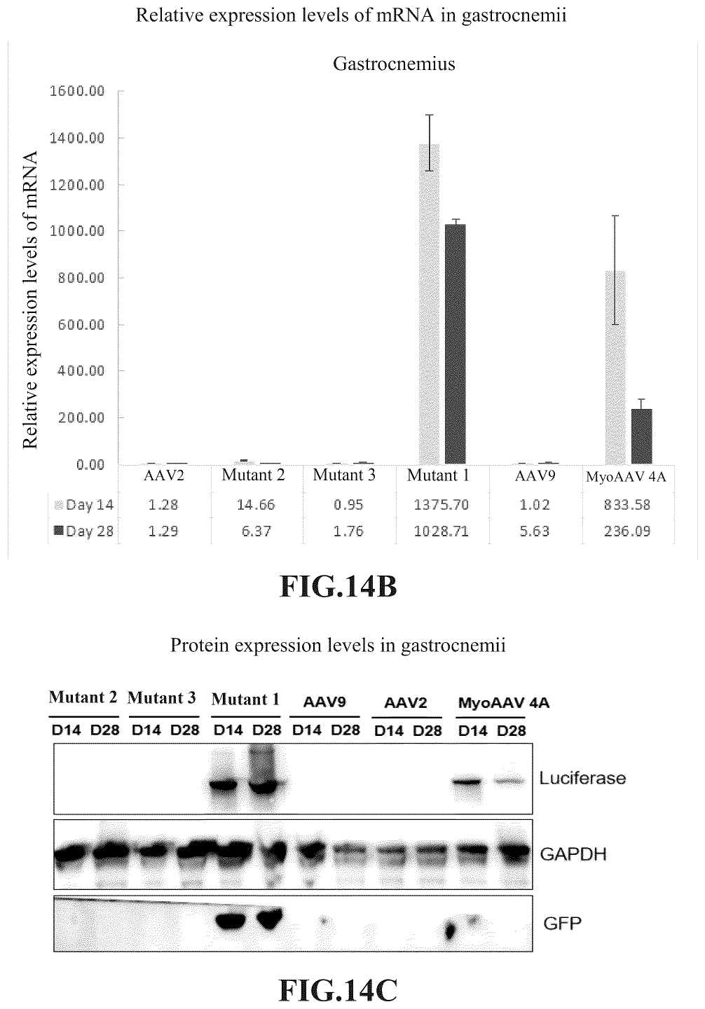

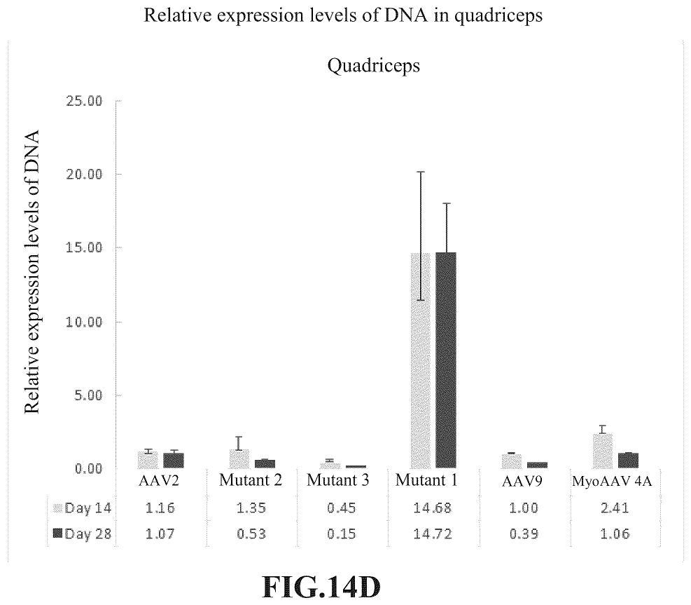

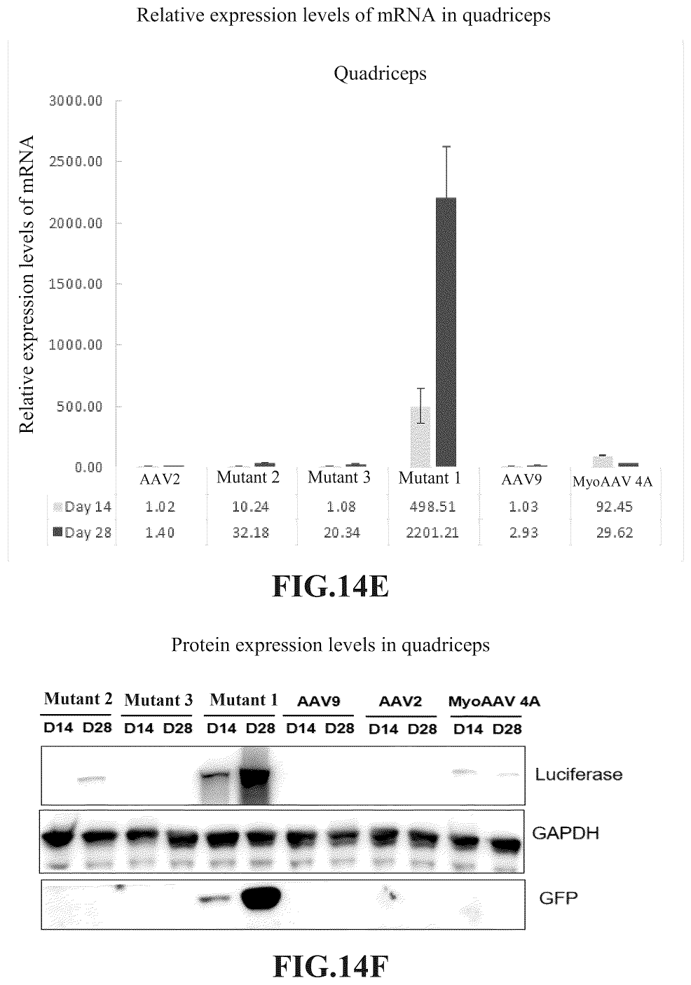

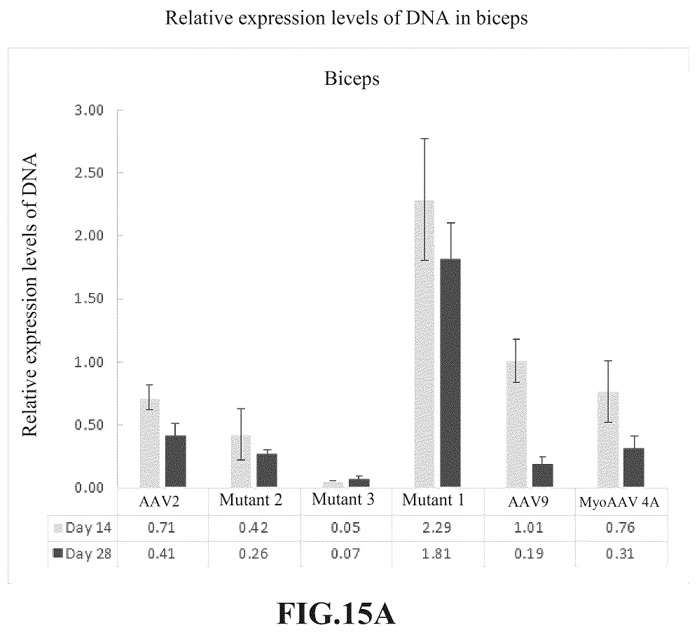

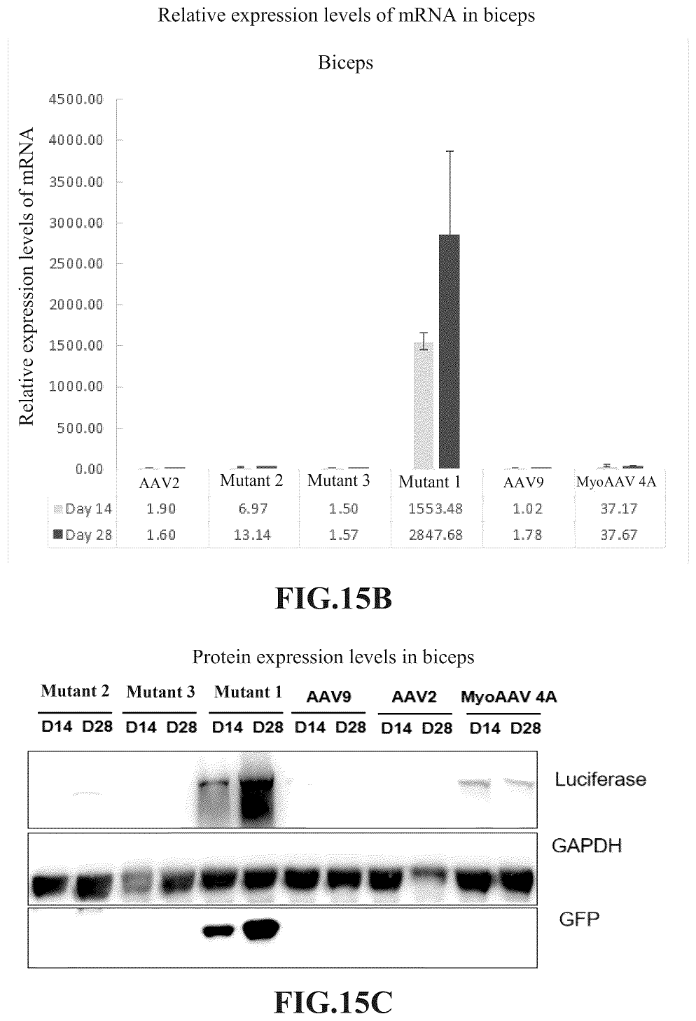

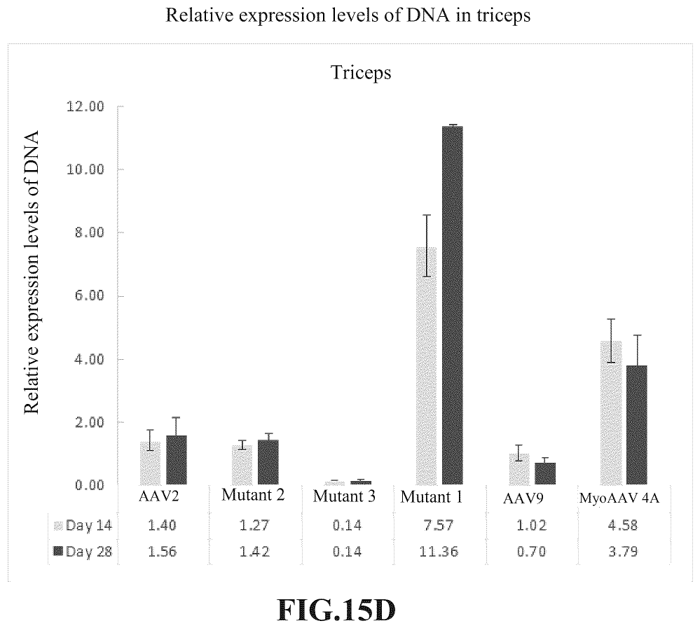

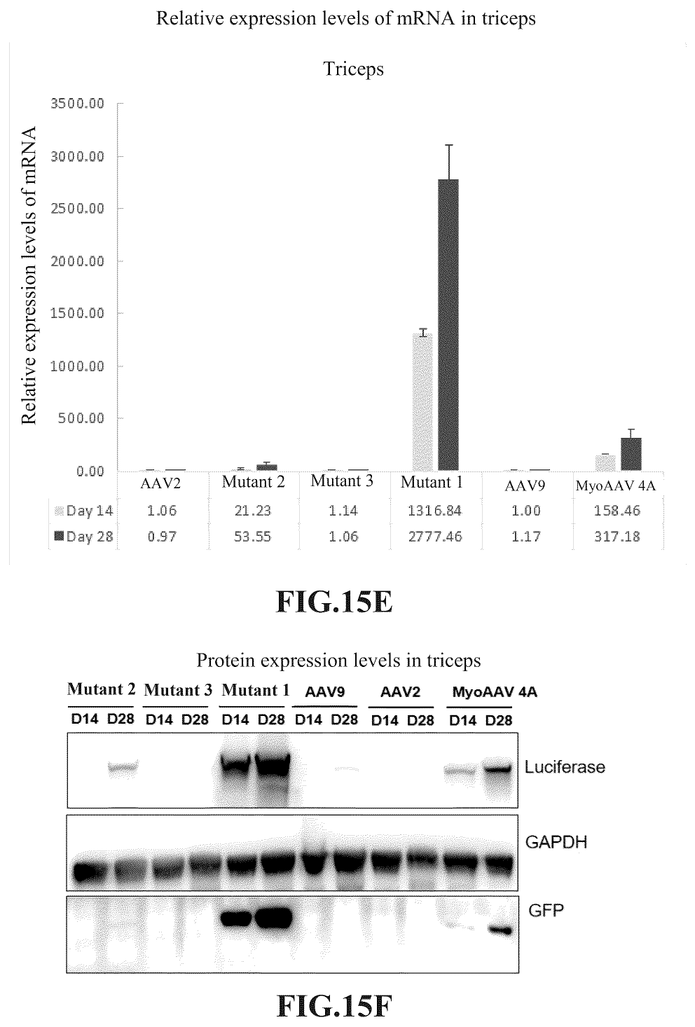

By analyzing muscle targeting of serotypes (FIGS. 14A-14F and FIGS. 15A-15F), we found completely different effects from effects in mice. In cynomolgus monkeys, mutant 1 demonstrates the best muscle targeting, while mutant 3, which shows better muscle targeting in mice, performs far inferior to mutant 1. 14 days after virus injection, the mRNA levels of mutant 1 in gastrocnemii, quadriceps, biceps, and triceps are 1375.7 times, 498.51 times, 1553.48 times, and 1316.84 times those of AAV9, respectively, and 1.65 times, 5.39 times, 41.79 times, and 8.31 times those of MyoAAV 4A. The mRNA levels of mutant 1 in gastrocnemii, quadriceps, biceps, and triceps (28 days after virus injection) are 4.36 times, 74.31 times, 75.6 times and 8.76 times those of MyoAAV 4A, respectively. The trends of DNA levels and protein expression of the mutants in cynomolgus monkeys are basically consistent with those of mRNA levels. Although mutant 2 retains muscle targeting, it has fallen far behind MyoAAV 4A, and mutant 3 has even lost its targeting function at multiple muscle tissue sites. While the underlying mechanism remains unclear, this partly illustrates the significant cross-species differences in serotypes (a phenomenon not uncommon in AAV serotype development). The final clinical application of AAV serotypes still requires evaluation in an animal model closely resembling humans. Therefore, in the experiment on cynomolgus monkeys, mutant 1 obtained by combining the RGD peptide with the novel backbone with low liver tropism we developed is optimal. Mutant 1 not only has stronger muscle targeting, but also shows greatly reduced liver toxicity, which has better specificity, thereby exhibiting greater clinical development potential and application prospects.

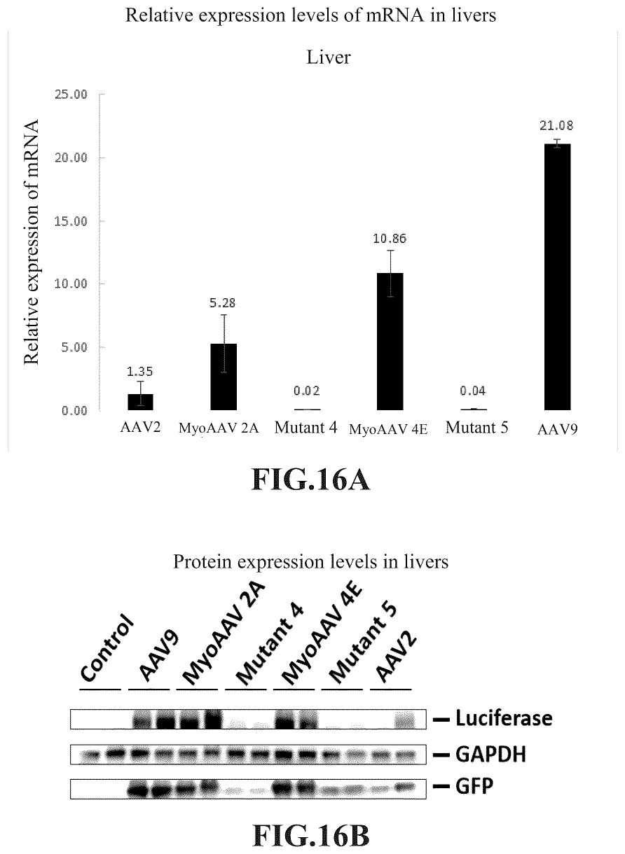

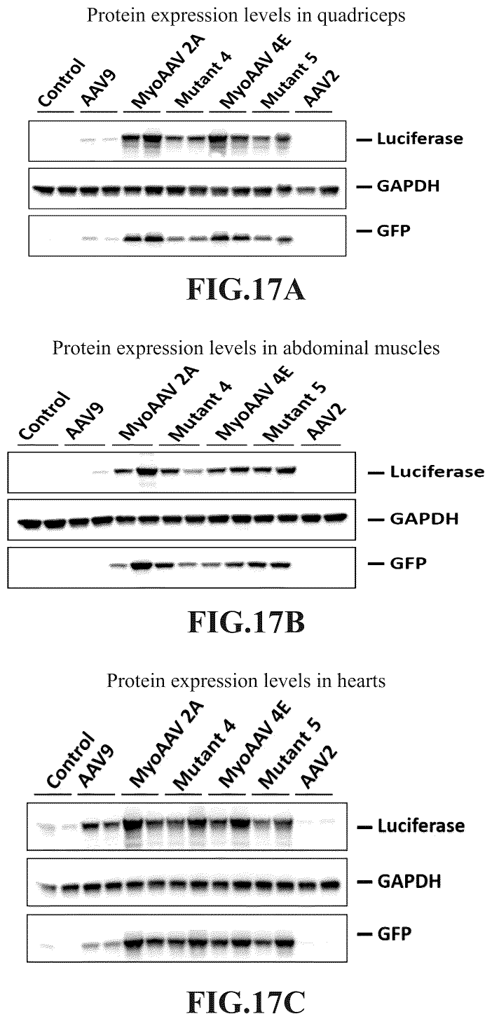

In view of the successful application of mutant 1, we expect that a simple and feasible approach will allow us to utilize existing targeting peptides and rapidly develop a variety of specific serotypes to meet the needs. Therefore, we designed mutants 4 and 5 and performed preliminary analysis of their functions. In mouse experiments, mutants 4 and 5 still show very low liver tropism (FIGS. 16A and 16B), which is much lower than that of AAV9. The liver tropism of MyoAAV 2A is about 264 times higher than that of mutant 4 (MyoAAV 2A and mutant 4 have the same RGD peptide), and the liver tropism of MyoAAV 4E is about 271.5 times higher than that of mutant 5 (MyoAAV 4E and mutant 5 have the same RGD peptide). By analyzing the protein expression levels in muscles (quadriceps, abdominal muscles) and hearts, we observed that although mutant 4 and mutant 5 exhibit superior muscle targeting compared to AAV9, their performance remains slightly lower than that of the controls MyoAAV 2A and MyoAAV 4E (FIGS. 17A-17C). The above results are similar to the effects of mutant 1 in mice, indicating the effectiveness of the vector backbones with low liver tropism. In short, in terms of the muscle/liver ratio, mutant 4 and mutant 5 have far surpassed MyoAAV 2A and MyoAAV 4E, and still have great advantages in practical application.

In summary, using the developed AAV serotypes with low liver tropism as the backbones, we have successfully developed AAV serotypes with low liver tropism and muscle targeting by constructing mutants of AAV capsid proteins via inserting different RGD peptides and performing in vivo analysis in mice and cynomolgus monkeys. Among them, mutant 1 is preferred as an AAV serotype having muscle targeting for clinical applications in non-human primates (NHPs) and humans. The innovation of this application is not only to provide various AAV serotypes with low liver tropism and muscle targeting through technological modifications, but also to discover the performance differences of different mutants of AAV capsid proteins across species through experiments on cynomolgus monkeys, and to find new AAV serotypes that are more suitable for clinical applications. This application circumvents the liver toxicity issues and high-dose issues required for muscle targeting that arise in current clinical trials, providing a safer, more reliable and affordable gene therapy product for the majority of patients.

In this application, we have developed AAVs with low hepatotoxicity and have explored their feasibility as a “backbone” platform. We introduced a class of muscle-targeting peptides containing the RGD motif into the AAV backbone having low toxicity, and relevant experimental studies from mice to non-human primates (NHPs) were conducted. Thus, AAVs with low liver tropism and better muscle specificity were developed. The AAVs provide better tool or carriers for drug delivery with clinical application value for a wide range of patients.

This application provides a variety of recombinant adeno-associated virus (rAAV) virions including mutant capsid proteins, and demonstrates the organ-targeting characteristics of these virions in mice and cynomolgus monkeys. The mutants provided in the present application exhibited the characteristic of low liver tropism in both species. Among them, mutant 1 has the best effects of low liver tropism and muscle targeting in cynomolgus monkeys, followed by mutant 2. Mutant 3 is more suitable for mouse application; in addition, mutant 4 and mutant 5 also show superior muscle/liver ratios in mice.

Finally, it should be noted that the above embodiments are only used to illustrate the technical solution of the present application and not to limit the scope of protection of the present application. Although the present application is described in detail with reference to the preferred embodiments, it should be understood by those of ordinary skill in the art that modifications or equivalent substitutions may be made to the technical solutions of the present application without departing from the substance and scope of the technical solutions of the present application.

Amino acid and nucleotide sequences referred to herein include the following.

SEQ ID NO: 1 (VP1 amino acid sequence of wild-type AAV2)
MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQAKKRVL
EPLGLVEEPVKTAPGKKRPVEHSPVEPDSSSGTGKAGQQPARKRLNFGQTGDADSVPDPQPLGQPPA
APSGLGTNTMATGSGAPMADNNEGADGVGNSSGNWHCDSTWMGDRVITTSTRTWALPTYNNHLY
KQISSQSGASNDNHYFGYSTPWGYFDFNRFHCHFSPRDWQRLINNNWGFRPKRLNFKLFNIQVKEVT
QNDGTTTIANNLTSTVQVFTDSEYQLPYVLGSAHQGCLPPFPADVFMVPQYGYLTLNNGSQAVGRSS
FYCLEYFPSQMLRTGNNFTFSYTFEDVPFHSSYAHSQSLDRLMNPLIDQYLYYLSRTNTPSGTTTQSRL
QFSQAGASDIRDQSRNWLPGPCYRQQRVSKTSADNNNSEYSWTGATKYHLNGRDSLVNPGPAMAS
HKDDEEKFFPQSGVLIFGKQGSEKTNVDIEKVMITDEEEIRTTNPVATEQYGSVSTNLQRGNRQAATA
DVNTQGVLPGMVWQDRDVYLQGPIWAKIPHTDGHFHPSPLMGGFGLKHPPPQILIKNTPVPANPSTT
FSAAKFASFITQYSTGQVSVEIEWELQKENSKRWNPEIQYTSNYNKSVNVDFTVDTNGVYSEPRPIGT
RYLTRNL*
SEQ ID NO: 2 (substitution sequence)
KTINGSGQNQQTLK
SEQ ID NO: 3 (substitution sequence)
TTVTQ
SEQ ID NO: 4 (substituted sequence)
RTNTPSGTTTQSRLQ
SEQ ID NO: 5 (substituted sequence)
KTSAD
SEQ ID NO: 6 (VP1 amino acid sequence of mutant 1′)
MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQAKKRVL
EPLGLVEEPVKTAPGKKRPVEHSPVEPDSSSGTGKAGQQPARKRLNFGQTGDADSVPDPQPLGQPPA
APSGLGTNTMATGSGAPMADNNEGADGVGNSSGNWHCDSTWMGDRVITTSTRTWALPTYNNHLY
KQISSQSGASNDNHYFGYSTPWGYFDFNRFHCHFSPRDWQRLINNNWGFRPKRLNFKLFNIQVKEVT
QNDGTTTIANNLTSTVQVFTDSEYQLPYVLGSAHQGCLPPFPADVFMVPQYGYLTLNNGSQAVGRSS
FYCLEYFPSQMLRTGNNFTFSYTFEDVPFHSSYAHSQSLDRLMNPLIDQYLYYLSKTINGSGQNQQTL
KFSQAGASDIRDQSRNWLPGPCYRQQRVSTTVTQNNNSEYSWTGATKYHLNGRDSLVNPGPAMAS
HKDDEEKFFPQSGVLIFGKQGSEKTNVDIEKVMITDEEEIRTTNPVATEQYGSVSTNLQRGNRQAATA
DVNTQGVLPGMVWQDRDVYLQGPIWAKIPHTDGHFHPSPLMGGFGLKHPPPQILIKNTPVPANPSTT
FSAAKFASFITQYSTGQVSVEIEWELQKENSKRWNPEIQYTSNYNKSVNVDFTVDINGVYSEPRPIGT
RYLTRNL*
SEQ ID NO: 7 (VP1 amino acid sequence of mutant 2′)
MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQAKKRVL
EPLGLVEEPVKTAPGKKRPVEHSPVEPDSSSGTGKAGQQPARKRLNFGQTGDADSVPDPQPLGQPPA
APSGLGTNTMATGSGAPMADNNEGADGVGNSSGNWHCDSTWMGDRVITTSTRTWALPTYNNHLY
KQISSQSGASNDNHYFGYSTPWGYFDFNRFHCHFSPRDWQRLINNNWGFRPKRLNFKLFNIQVKEVT
QNDGTTTIANNLTSTVQVFTDSEYQLPYVLGSAHQGCLPPFPADVFMVPQYGYLTLNNGSQAVGRSS
FYCLEYFPSQMLRTGNNFTFSYTFEDVPFHSSYAHSQSLDRLMNPLIDQYLYYLSKTINGSGQNQQTL
KFSQAGASDIRDQSRNWLPGPCYRQQRVSTTVTQNNNSEYSWTGATKYHLNGRDSLVNPGPAMAS
HKDDEEKFFPQSGVLIFGKQGSEKTNVDIEKVMITDEEEIRTTNPVATEQYGSVSTNLQAGNRQAATA
DVNTQGVLPGMVWQDRDVYLQGPIWAKIPHTDGHFHPSPLMGGFGLKHPPPQILIKNTPVPANPSTT
FSAAKFASFITQYSTGQVSVEIEWELQKENSKRWNPEIQYTSNYNKSVNVDFTVDTNGVYSEPRPIGT
RYLTRNL*
SEQ ID NO: 8 (VP1 amino acid sequence of mutant 3′)
MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQAKKRVL
EPLGLVEEPVKTAPGKKRPVEHSPVEPDSSSGTGKAGQQPARKRLNFGQTGDADSVPDPQPLGQPPA
APSGLGTNTMATGSGAPMADNNEGADGVGNSSGNWHCDSTWMGDRVITTSTRTWALPTYNNHLY
KQISSQSGASNDNHYFGYSTPWGYFDFNRFHCHFSPRDWQRLINNNWGFRPKRLNFKLFNIQVKEVT
QNDGTTTIANNLTSTVQVFTDSEYQLPYVLGSAHQGCLPPFPADVFMVPQYGYLTLNNGSQAVGRSS
FYCLEYFPSQMLRTGNNFTFSYTFEDVPFHSSYAHSQSLDRLMNPLIDQYLYYLSKTINGSGQNQQTL
KFSQAGASDIRDQSRNWLPGPCYRQQRVSTTVTQNNNSEYSWTGATKYHLNGRDSLVNPGPAMAS
HKDDEEKFFPQSGVLIFGKQGSEKTNVDIEKVMITDEEEIRTTNPVATEQYGSVSTNLQRQAATADVN
TQGVLPGMVWQDRDVYLQGPIWAKIPHTDGHFHPSPLMGGFGLKHPPPQILIKNTPVPANPSTTFSAA
KFASFITQYSTGQVSVEIEWELQKENSKRWNPEIQYTSNYNKSVNVDFTVDTNGVYSEPRPIGTRYLT
RNL*
SEQ ID NO: 9 (VP1 nucleic acid sequence of AAV2 (5′ → 3′))
ATGGCTGCCGATGGTTATCTTCCAGATTGGCTCGAGGACACTCTCTCTGAAGGAATAAGA
CAGTGGTGGAAGCTCAAACCTGGCCCACCACCACCAAAGCCCGCAGAGCGGCATAAGGACGAC
AGCAGGGGTCTTGTGCTTCCTGGGTACAAGTACCTCGGACCCTTCAACGGACTCGACAAGGGAG
AGCCGGTCAACGAGGCAGACGCCGCGGCCCTCGAGCACGACAAAGCCTACGACCGGCAGCTCG
ACAGCGGAGACAACCCGTACCTCAAGTACAACCACGCCGACGCGGAGTTTCAGGAGCGCCTTAA
AGAAGATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTAGAGC
ACTCTCCTGTGGAGCCAGACTCCTCCTCGGGAACCGGAAAGGCGGGCCAGCAGCCTGCAAGAAA
AAGATTGAATTTTGGTCAGACTGGAGACGCAGACTCAGTACCTGACCCCCAGCCTCTCGGACAG
CCACCAGCAGCCCCCTCTGGTCTGGGAACTAATACGATGGCTACAGGCAGTGGCGCACCAATGG
CAGACAATAACGAGGGCGCCGACGGAGTGGGTAATTCCTCGGGAAATTGGCATTGCGATTCCAC
ATGGATGGGCGACAGAGTCATCACCACCAGCACCCGAACCTGGGCCCTGCCCACCTACAACAAC
CACCTCTACAAACAAATTTCCAGCCAATCAGGAGCCTCGAACGACAATCACTACTTTGGCTACAG
CACCCCTTGGGGGTATTTTGACTTCAACAGATTCCACTGCCACTTTTCACCACGTGACTGGCAAA
GACTCATCAACAACAACTGGGGATTCCGACCCAAGAGACTCAACTTCAAGCTCTTTAACATTCAA
GTCAAAGAGGTCACGCAGAATGACGGTACGACGACGATTGCCAATAACCTTACCAGCACGGTTC
AGGTGTTTACTGACTCGGAGTACCAGCTCCCGTACGTCCTCGGCTCGGCGCATCAAGGATGCCTC
CCGCCGTTCCCAGCAGACGTCTTCATGGTGCCACAGTATGGATACCTCACCCTGAACAACGGGA
GTCAGGCAGTAGGACGCTCTTCATTTTACTGCCTGGAGTACTTTCCTTCTCAGATGCTGCGTACCG
GAAACAACTTTACCTTCAGCTACACTTTTGAGGACGTTCCTTTCCACAGCAGCTACGCTCACAGC
CAGAGTCTGGACCGTCTCATGAATCCTCTCATCGACCAGTACCTGTATTACTTGAGCAGAACAAA
CACTCCAAGTGGAACCACCACGCAGTCAAGGCTTCAGTTTTCTCAGGCCGGAGCGAGTGACATT
CGGGACCAGTCTAGGAACTGGCTTCCTGGACCCTGTTACCGCCAGCAGCGAGTATCAAAGACAT
CTGCGGATAACAACAACAGTGAATACTCGTGGACTGGAGCTACCAAGTACCACCTCAATGGCAG
AGACTCTCTGGTGAATCCGGGCCCGGCCATGGCAAGCCACAAGGACGATGAAGAAAAGTTTTTT
CCTCAGAGCGGGGTTCTCATCTTTGGGAAGCAAGGCTCAGAGAAAACAAATGTGGACATTGAAA
AGGTCATGATTACAGACGAAGAGGAAATCAGGACAACCAATCCCGTGGCTACGGAGCAGTATG
GTTCTGTATCTACCAACCTCCAGAGAGGCAACAGACAAGCAGCTACCGCAGATGTCAACACACA
AGGCGTTCTTCCAGGCATGGTCTGGCAGGACAGAGATGTGTACCTTCAGGGGCCCATCTGGGCA
AAGATTCCACACACGGACGGACATTTTCACCCCTCTCCCCTCATGGGTGGATTCGGACTTAAACA
CCCTCCTCCACAGATTCTCATCAAGAACACCCCGGTACCTGCGAATCCTTCGACCACCTTCAGTG
CGGCAAAGTTTGCTTCCTTCATCACACAGTACTCCACGGGACAGGTCAGCGTGGAGATCGAGTG
GGAGCTGCAGAAGGAAAACAGCAAACGCTGGAATCCCGAAATTCAGTACACTTCCAACTACAAC
AAGTCTGTTAATGTGGACTTTACTGTGGACACTAATGGCGTGTATTCAGAGCCTCGCCCCATTGG
CACCAGATACCTGACTCGTAATCTGTAA
SEQ ID NO: 10 (VP1 nucleic acid sequence of mutant 1′(5′ → 3′))
ATGGCTGCCGATGGTTATCTTCCAGATTGGCTCGAGGACACTCTCTCTGAAGGAATAAGA
CAGTGGTGGAAGCTCAAACCTGGCCCACCACCACCAAAGCCCGCAGAGCGGCATAAGGACGAC
AGCAGGGGTCTTGTGCTTCCTGGGTACAAGTACCTCGGACCCTTCAACGGACTCGACAAGGGAG
AGCCGGTCAACGAGGCAGACGCCGCGGCCCTCGAGCACGACAAAGCCTACGACCGGCAGCTCG
ACAGCGGAGACAACCCGTACCTCAAGTACAACCACGCCGACGCGGAGTTTCAGGAGCGCCTTAA
AGAAGATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTAGAGC
ACTCTCCTGTGGAGCCAGACTCCTCCTCGGGAACCGGAAAGGCGGGCCAGCAGCCTGCAAGAAA
AAGATTGAATTTTGGTCAGACTGGAGACGCAGACTCAGTACCTGACCCCCAGCCTCTCGGACAG
CCACCAGCAGCCCCCTCTGGTCTGGGAACTAATACGATGGCTACAGGCAGTGGCGCACCAATGG
CAGACAATAACGAGGGCGCCGACGGAGTGGGTAATTCCTCGGGAAATTGGCATTGCGATTCCAC
ATGGATGGGCGACAGAGTCATCACCACCAGCACCCGAACCTGGGCCCTGCCCACCTACAACAAC
CACCTCTACAAACAAATTTCCAGCCAATCAGGAGCCTCGAACGACAATCACTACTTTGGCTACAG
CACCCCTTGGGGGTATTTTGACTTCAACAGATTCCACTGCCACTTTTCACCACGTGACTGGCAAA
GACTCATCAACAACAACTGGGGATTCCGACCCAAGAGACTCAACTTCAAGCTCTTTAACATTCAA
GTCAAAGAGGTCACGCAGAATGACGGTACGACGACGATTGCCAATAACCTTACCAGCACGGTTC
AGGTGTTTACTGACTCGGAGTACCAGCTCCCGTACGTCCTCGGCTCGGCGCATCAAGGATGCCTC
CCGCCGTTCCCAGCAGACGTCTTCATGGTGCCACAGTATGGATACCTCACCCTGAACAACGGGA
GTCAGGCAGTAGGACGCTCTTCATTTTACTGCCTGGAGTACTTTCCTTCTCAGATGCTGCGTACCG
GAAACAACTTTACCTTCAGCTACACTTTTGAGGACGTTCCTTTCCACAGCAGCTACGCTCACAGC
CAGAGTCTGGACCGTCTCATGAATCCTCTCATCGACCAGTACCTGTATTACTTGAGCAAGACTAT
TAACGGTTCTGGACAGAATCAACAAACGCTAAAATTTTCTCAGGCCGGAGCGAGTGACATTCGG
GACCAGTCTAGGAACTGGCTTCCTGGACCCTGTTACCGCCAGCAGCGAGTATCAACCACTGTGAC
TCAAAACAACAACAGTGAATACTCGTGGACTGGAGCTACCAAGTACCACCTCAATGGCAGAGAC
TCTCTGGTGAATCCGGGCCCGGCCATGGCAAGCCACAAGGACGATGAAGAAAAGTTTTTTCCTC
AGAGCGGGGTTCTCATCTTTGGGAAGCAAGGCTCAGAGAAAACAAATGTGGACATTGAAAAGGT
CATGATTACAGACGAAGAGGAAATCAGGACAACCAATCCCGTGGCTACGGAGCAGTATGGTTCT
GTATCTACCAACCTCCAGAGAGGCAACAGACAAGCAGCTACCGCAGATGTCAACACACAAGGCG
TTCTTCCAGGCATGGTCTGGCAGGACAGAGATGTGTACCTTCAGGGGCCCATCTGGGCAAAGATT
CCACACACGGACGGACATTTTCACCCCTCTCCCCTCATGGGTGGATTCGGACTTAAACACCCTCC
TCCACAGATTCTCATCAAGAACACCCCGGTACCTGCGAATCCTTCGACCACCTTCAGTGCGGCAA
AGTTTGCTTCCTTCATCACACAGTACTCCACGGGACAGGTCAGCGTGGAGATCGAGTGGGAGCT
GCAGAAGGAAAACAGCAAACGCTGGAATCCCGAAATTCAGTACACTTCCAACTACAACAAGTCT
GTTAATGTGGACTTTACTGTGGACACTAATGGCGTGTATTCAGAGCCTCGCCCCATTGGCACCAG
ATACCTGACTCGTAATCTGTAA
SEQ ID NO: 11 (VP1 nucleic acid sequence of mutant 2′(5′ → 3′))
ATGGCTGCCGATGGTTATCTTCCAGATTGGCTCGAGGACACTCTCTCTGAAGGAATAAGA
CAGTGGTGGAAGCTCAAACCTGGCCCACCACCACCAAAGCCCGCAGAGCGGCATAAGGACGAC
AGCAGGGGTCTTGTGCTTCCTGGGTACAAGTACCTCGGACCCTTCAACGGACTCGACAAGGGAG
AGCCGGTCAACGAGGCAGACGCCGCGGCCCTCGAGCACGACAAAGCCTACGACCGGCAGCTCG
ACAGCGGAGACAACCCGTACCTCAAGTACAACCACGCCGACGCGGAGTTTCAGGAGCGCCTTAA
AGAAGATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTAGAGC
ACTCTCCTGTGGAGCCAGACTCCTCCTCGGGAACCGGAAAGGCGGGCCAGCAGCCTGCAAGAAA
AAGATTGAATTTTGGTCAGACTGGAGACGCAGACTCAGTACCTGACCCCCAGCCTCTCGGACAG
CCACCAGCAGCCCCCTCTGGTCTGGGAACTAATACGATGGCTACAGGCAGTGGCGCACCAATGG
CAGACAATAACGAGGGCGCCGACGGAGTGGGTAATTCCTCGGGAAATTGGCATTGCGATTCCAC
ATGGATGGGCGACAGAGTCATCACCACCAGCACCCGAACCTGGGCCCTGCCCACCTACAACAAC
CACCTCTACAAACAAATTTCCAGCCAATCAGGAGCCTCGAACGACAATCACTACTTTGGCTACAG
CACCCCTTGGGGGTATTTTGACTTCAACAGATTCCACTGCCACTTTTCACCACGTGACTGGCAAA
GACTCATCAACAACAACTGGGGATTCCGACCCAAGAGACTCAACTTCAAGCTCTTTAACATTCAA
GTCAAAGAGGTCACGCAGAATGACGGTACGACGACGATTGCCAATAACCTTACCAGCACGGTTC
AGGTGTTTACTGACTCGGAGTACCAGCTCCCGTACGTCCTCGGCTCGGCGCATCAAGGATGCCTC
CCGCCGTTCCCAGCAGACGTCTTCATGGTGCCACAGTATGGATACCTCACCCTGAACAACGGGA
GTCAGGCAGTAGGACGCTCTTCATTTTACTGCCTGGAGTACTTTCCTTCTCAGATGCTGCGTACCG
GAAACAACTTTACCTTCAGCTACACTTTTGAGGACGTTCCTTTCCACAGCAGCTACGCTCACAGC
CAGAGTCTGGACCGTCTCATGAATCCTCTCATCGACCAGTACCTGTATTACTTGAGCAAGACTAT
TAACGGTTCTGGACAGAATCAACAAACGCTAAAATTTTCTCAGGCCGGAGCGAGTGACATTCGG
GACCAGTCTAGGAACTGGCTTCCTGGACCCTGTTACCGCCAGCAGCGAGTATCAACCACTGTGAC
TCAAAACAACAACAGTGAATACTCGTGGACTGGAGCTACCAAGTACCACCTCAATGGCAGAGAC
TCTCTGGTGAATCCGGGCCCGGCCATGGCAAGCCACAAGGACGATGAAGAAAAGTTTTTTCCTC
AGAGCGGGGTTCTCATCTTTGGGAAGCAAGGCTCAGAGAAAACAAATGTGGACATTGAAAAGGT
CATGATTACAGACGAAGAGGAAATCAGGACAACCAATCCCGTGGCTACGGAGCAGTATGGTTCT
GTATCTACCAACCTCCAGGCTGGCAACAGACAAGCAGCTACCGCAGATGTCAACACACAAGGCG
TTCTTCCAGGCATGGTCTGGCAGGACAGAGATGTGTACCTTCAGGGGCCCATCTGGGCAAAGATT
CCACACACGGACGGACATTTTCACCCCTCTCCCCTCATGGGTGGATTCGGACTTAAACACCCTCC
TCCACAGATTCTCATCAAGAACACCCCGGTACCTGCGAATCCTTCGACCACCTTCAGTGCGGCAA
AGTTTGCTTCCTTCATCACACAGTACTCCACGGGACAGGTCAGCGTGGAGATCGAGTGGGAGCT
GCAGAAGGAAAACAGCAAACGCTGGAATCCCGAAATTCAGTACACTTCCAACTACAACAAGTCT
GTTAATGTGGACTTTACTGTGGACACTAATGGCGTGTATTCAGAGCCTCGCCCCATTGGCACCAG
ATACCTGACTCGTAATCTGTAA
SEQ ID NO: 12 (VP1 nucleic acid sequence of mutant 3′(5′ → 3′))
ATGGCTGCCGATGGTTATCTTCCAGATTGGCTCGAGGACACTCTCTCTGAAGGAATAAGACAGTG
GTGGAAGCTCAAACCTGGCCCACCACCACCAAAGCCCGCAGAGCGGCATAAGGACGACAGCAG
GGGTCTTGTGCTTCCTGGGTACAAGTACCTCGGACCCTTCAACGGACTCGACAAGGGAGAGCCG
GTCAACGAGGCAGACGCCGCGGCCCTCGAGCACGACAAAGCCTACGACCGGCAGCTCGACAGC
GGAGACAACCCGTACCTCAAGTACAACCACGCCGACGCGGAGTTTCAGGAGCGCCTTAAAGAAG
ATACGTCTTTTGGGGGCAACCTCGGACGAGCAGTCTTCCAGGCGAAAAAGAGGGTTCTTGAACC
TCTGGGCCTGGTTGAGGAACCTGTTAAGACGGCTCCGGGAAAAAAGAGGCCGGTAGAGCACTCT
CCTGTGGAGCCAGACTCCTCCTCGGGAACCGGAAAGGCGGGCCAGCAGCCTGCAAGAAAAAGA
TTGAATTTTGGTCAGACTGGAGACGCAGACTCAGTACCTGACCCCCAGCCTCTCGGACAGCCACC
AGCAGCCCCCTCTGGTCTGGGAACTAATACGATGGCTACAGGCAGTGGCGCACCAATGGCAGAC
AATAACGAGGGCGCCGACGGAGTGGGTAATTCCTCGGGAAATTGGCATTGCGATTCCACATGGA
TGGGCGACAGAGTCATCACCACCAGCACCCGAACCTGGGCCCTGCCCACCTACAACAACCACCT
CTACAAACAAATTTCCAGCCAATCAGGAGCCTCGAACGACAATCACTACTTTGGCTACAGCACC
CCTTGGGGGTATTTTGACTTCAACAGATTCCACTGCCACTTTTCACCACGTGACTGGCAAAGACT
CATCAACAACAACTGGGGATTCCGACCCAAGAGACTCAACTTCAAGCTCTTTAACATTCAAGTCA
AAGAGGTCACGCAGAATGACGGTACGACGACGATTGCCAATAACCTTACCAGCACGGTTCAGGT
GTTTACTGACTCGGAGTACCAGCTCCCGTACGTCCTCGGCTCGGCGCATCAAGGATGCCTCCCGC
CGTTCCCAGCAGACGTCTTCATGGTGCCACAGTATGGATACCTCACCCTGAACAACGGGAGTCA
GGCAGTAGGACGCTCTTCATTTTACTGCCTGGAGTACTTTCCTTCTCAGATGCTGCGTACCGGAA
ACAACTTTACCTTCAGCTACACTTTTGAGGACGTTCCTTTCCACAGCAGCTACGCTCACAGCCAG
AGTCTGGACCGTCTCATGAATCCTCTCATCGACCAGTACCTGTATTACTTGAGCAAGACTATTAA
CGGTTCTGGACAGAATCAACAAACGCTAAAATTTTCTCAGGCCGGAGCGAGTGACATTCGGGAC
CAGTCTAGGAACTGGCTTCCTGGACCCTGTTACCGCCAGCAGCGAGTATCAACCACTGTGACTCA
AAACAACAACAGTGAATACTCGTGGACTGGAGCTACCAAGTACCACCTCAATGGCAGAGACTCT
CTGGTGAATCCGGGCCCGGCCATGGCAAGCCACAAGGACGATGAAGAAAAGTTTTTTCCTCAGA
GCGGGGTTCTCATCTTTGGGAAGCAAGGCTCAGAGAAAACAAATGTGGACATTGAAAAGGTCAT
GATTACAGACGAAGAGGAAATCAGGACAACCAATCCCGTGGCTACGGAGCAGTATGGTTCTGTA
TCTACCAACCTCCAGAGACAAGCAGCTACCGCAGATGTCAACACACAAGGCGTTCTTCCAGGCA
TGGTCTGGCAGGACAGAGATGTGTACCTTCAGGGGCCCATCTGGGCAAAGATTCCACACACGGA
CGGACATTTTCACCCCTCTCCCCTCATGGGTGGATTCGGACTTAAACACCCTCCTCCACAGATTCT
CATCAAGAACACCCCGGTACCTGCGAATCCTTCGACCACCTTCAGTGCGGCAAAGTTTGCTTCCT
TCATCACACAGTACTCCACGGGACAGGTCAGCGTGGAGATCGAGTGGGAGCTGCAGAAGGAAA
ACAGCAAACGCTGGAATCCCGAAATTCAGTACACTTCCAACTACAACAAGTCTGTTAATGTGGA
CTTTACTGTGGACACTAATGGCGTGTATTCAGAGCCTCGCCCCATTGGCACCAGATACCTGACTC
GTAATCTGTAA
SEQ ID NO: 27 (VP1 amino acid sequence of mutant 1)
MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQAKKRVL
EPLGLVEEPVKTAPGKKRPVEHSPVEPDSSSGTGKAGQQPARKRLNFGQTGDADSVPDPQPLGQPPA
APSGLGTNTMATGSGAPMADNNEGADGVGNSSGNWHCDSTWMGDRVITTSTRTWALPTYNNHLY
KQISSQSGASNDNHYFGYSTPWGYFDFNRFHCHFSPRDWQRLINNNWGFRPKRLNFKLFNIQVKEVT
QNDGTTTIANNLTSTVQVFTDSEYQLPYVLGSAHQGCLPPFPADVFMVPQYGYLTLNNGSQAVGRSS
FYCLEYFPSQMLRTGNNFTFSYTFEDVPFHSSYAHSQSLDRLMNPLIDQYLYYLSKTINGSGQNQQTL
KFSQAGASDIRDQSRNWLPGPCYRQQRVSTTVTQNNNSEYSWTGATKYHLNGRDSLVNPGPAMAS
HKDDEEKFFPQSGVLIFGKQGSEKTNVDIEKVMITDEEEIRTTNPVATEQYGSVSTNLQSNSRGDYNS
LRQAATADVNTQGVLPGMVWQDRDVYLQGPIWAKIPHTDGHFHPSPLMGGFGLKHPPPQILIKNTP
VPANPSTTFSAAKFASFITQYSTGQVSVEIEWELQKENSKRWNPEIQYTSNYNKSVNVDFTVDTNGV
YSEPRPIGTRYLTRNL*
SEQ ID NO: 28 (VP1 amino acid sequence of mutant 2)
MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQAKKRVL
EPLGLVEEPVKTAPGKKRPVEHSPVEPDSSSGTGKAGQQPARKRLNFGQTGDADSVPDPQPLGQPPA
APSGLGTNTMATGSGAPMADNNEGADGVGNSSGNWHCDSTWMGDRVITTSTRTWALPTYNNHLY
KQISSQSGASNDNHYFGYSTPWGYFDFNRFHCHFSPRDWQRLINNNWGFRPKRLNFKLFNIQVKEVT
QNDGTTTIANNLTSTVQVFTDSEYQLPYVLGSAHQGCLPPFPADVFMVPQYGYLTLNNGSQAVGRSS
FYCLEYFPSQMLRTGNNFTFSYTFEDVPFHSSYAHSQSLDRLMNPLIDQYLYYLSRTNTPSGTTTQSRL
QFSQAGASDIRDQSRNWLPGPCYRQQRVSKTSADNNNSEYSWTGATKYHLNGRDSLVNPGPAMAS
HKDDEEKFFPQSGVLIFGKQGSEKTNVDIEKVMITDEEEIRTTNPVATEQYGSVSTNLQSNSRGDYNS
LRQAATADVNTQGVLPGMVWQDRDVYLQGPIWAKIPHTDGHFHPSPLMGGFGLKHPPPQILIKNTP
VPANPSTTFSAAKFASFITQYSTGQVSVEIEWELQKENSKRWNPEIQYTSNYNKSVNVDFTVDTNGV
YSEPRPIGTRYLTRNL*
SEQ ID NO: 29 (VP1 amino acid sequence of mutant 3)
MAADGYLPDWLEDTLSEGIRQWWKLKPGPPPPKPAERHKDDSRGLVLPGYKYLGPFNGLDKGEPVN
EADAAALEHDKAYDRQLDSGDNPYLKYNHADAEFQERLKEDTSFGGNLGRAVFQAKKRVLEPLGL
VEEPVKTAPGKKRPVEHSPVEPDSSSGTGKAGQQPARKRLNFGQTGDADSVPDPQPLGQPPAAPSGL
GTNTMATGSGAPMADNNEGADGVGNSSGNWHCDSTWMGDRVITTSTRTWALPTYNNHLYKQISSQ
SGASNDNHYFGYSTPWGYFDFNRFHCHFSPRDWQRLINNNWGFRPKRLNFKLFNIQVKEVTQNDGT
TTIANNLTSTVQVFTDSEYQLPYVLGSAHQGCLPPFPADVFMVPQYGYLTLNNGSQAVGRSSFYCLE
YFPSQMLRTGNNFTFSYTFEDVPFHSSYAHSQSLDRLMNPLIDQYLYYLSKTINGSGQNQQTLKFSQA
GASDIRDQSRNWLPGPCYRQQRVSKTSADNNNSEYSWTGATKYHLNGRDSLVNPGPAMASHKDDE
EKFFPQSGVLIFGKQGSEKTNVDIEKVMITDEEEIRTTNPVATEQYGSVSTNLQSNSRGDYNSLRQAA
TADVNTQGVLPGMVWQDRDVYLQGPIWAKIPHTDGHFHPSPLMGGFGLKHPPPQILIKNTPVPANPS
TTFSAAKFASFITQYSTGQVSVEIEWELQKENSKRWNPEIQYTSNYNKSVNVDFTVDTNGVYSEPRPI
GTRYLTRNL*
SEQ ID NO: 30 (VP1 amino acid sequence of mutant 4)
MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQAKKRVL
EPLGLVEEPVKTAPGKKRPVEHSPVEPDSSSGTGKAGQQPARKRLNFGQTGDADSVPDPQPLGQPPA
APSGLGTNTMATGSGAPMADNNEGADGVGNSSGNWHCDSTWMGDRVITTSTRTWALPTYNNHLY
KQISSQSGASNDNHYFGYSTPWGYFDFNRFHCHFSPRDWQRLINNNWGFRPKRLNFKLFNIQVKEVT
QNDGTTTIANNLTSTVQVFTDSEYQLPYVLGSAHQGCLPPFPADVFMVPQYGYLTLNNGSQAVGRSS
FYCLEYFPSQMLRTGNNFTFSYTFEDVPFHSSYAHSQSLDRLMNPLIDQYLYYLSKTINGSGQNQQTL
KFSQAGASDIRDQSRNWLPGPCYRQQRVSTTVTQNNNSEYSWTGATKYHLNGRDSLVNPGPAMAS
HKDDEEKFFPQSGVLIFGKQGSEKTNVDIEKVMITDEEEIRTTNPVATEQYGSVSTNLQGPGRGDQTT
LRQAATADVNTQGVLPGMVWQDRDVYLQGPIWAKIPHTDGHFHPSPLMGGFGLKHPPPQILIKNTP
VPANPSTTFSAAKFASFITQYSTGQVSVEIEWELQKENSKRWNPEIQYTSNYNKSVNVDFTVDTNGV
YSEPRPIGTRYLTRNL*
SEQ ID NO: 31 (VP1 amino acid sequence of mutant 5)
MAADGYLPDWLEDTLSEGIRQWWKLKPGPPPPKPAERHKDDSRGLVLPGYKYLGPFNGLDK
GEPVNEADAAALEHDKAYDRQLDSGDNPYLKYNHADAEFQERLKEDTSFGGNLGRAVFQAKKRVL
EPLGLVEEPVKTAPGKKRPVEHSPVEPDSSSGTGKAGQQPARKRLNFGQTGDADSVPDPQPLGQPPA
APSGLGTNTMATGSGAPMADNNEGADGVGNSSGNWHCDSTWMGDRVITTSTRTWALPTYNNHLY
KQISSQSGASNDNHYFGYSTPWGYFDFNRFHCHFSPRDWQRLINNNWGFRPKRLNFKLFNIQVKEVT
QNDGTTTIANNLTSTVQVFTDSEYQLPYVLGSAHQGCLPPFPADVFMVPQYGYLTLNNGSQAVGRSS
FYCLEYFPSQMLRTGNNFTFSYTFEDVPFHSSYAHSQSLDRLMNPLIDQYLYYLSKTINGSGQNQQTL
KFSQAGASDIRDQSRNWLPGPCYRQQRVSTTVTQNNNSEYSWTGATKYHLNGRDSLVNPGPAMAS
HKDDEEKFFPQSGVLIFGKQGSEKTNVDIEKVMITDEEEIRTTNPVATEQYGSVSTNLQENRRGDFNN
TRQAATADVNTQGVLPGMVWQDRDVYLQGPIWAKIPHTDGHFHPSPLMGGFGLKHPPPQILIKNTP
VPANPSTTFSAAKFASFITQYSTGQVSVEIEWELQKENSKRWNPEIQYTSNYNKSVNVDFTVDTNGV
YSEPRPIGTRYLTRNL*
SEQ ID NO: 32 (VP1 nucleic acid sequence of mutant 1 (5′ → 3′))
ATGGCTGCCGATGGTTATCTTCCAGATTGGCTCGAGGACACTCTCTCTGAAGGAATAAGA
CAGTGGTGGAAGCTCAAACCTGGCCCACCACCACCAAAGCCCGCAGAGCGGCATAAGGACGAC
AGCAGGGGTCTTGTGCTTCCTGGGTACAAGTACCTCGGACCCTTCAACGGACTCGACAAGGGAG
AGCCGGTCAACGAGGCAGACGCCGCGGCCCTCGAGCACGACAAAGCCTACGACCGGCAGCTCG
ACAGCGGAGACAACCCGTACCTCAAGTACAACCACGCCGACGCGGAGTTTCAGGAGCGCCTTAA
AGAAGATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTAGAGC
ACTCTCCTGTGGAGCCAGACTCCTCCTCGGGAACCGGAAAGGCGGGCCAGCAGCCTGCAAGAAA
AAGATTGAATTTTGGTCAGACTGGAGACGCAGACTCAGTACCTGACCCCCAGCCTCTCGGACAG
CCACCAGCAGCCCCCTCTGGTCTGGGAACTAATACGATGGCTACAGGCAGTGGCGCACCAATGG
CAGACAATAACGAGGGCGCCGACGGAGTGGGTAATTCCTCGGGAAATTGGCATTGCGATTCCAC
ATGGATGGGCGACAGAGTCATCACCACCAGCACCCGAACCTGGGCCCTGCCCACCTACAACAAC
CACCTCTACAAACAAATTTCCAGCCAATCAGGAGCCTCGAACGACAATCACTACTTTGGCTACAG
CACCCCTTGGGGGTATTTTGACTTCAACAGATTCCACTGCCACTTTTCACCACGTGACTGGCAAA
GACTCATCAACAACAACTGGGGATTCCGACCCAAGAGACTCAACTTCAAGCTCTTTAACATTCAA
GTCAAAGAGGTCACGCAGAATGACGGTACGACGACGATTGCCAATAACCTTACCAGCACGGTTC
AGGTGTTTACTGACTCGGAGTACCAGCTCCCGTACGTCCTCGGCTCGGCGCATCAAGGATGCCTC
CCGCCGTTCCCAGCAGACGTCTTCATGGTGCCACAGTATGGATACCTCACCCTGAACAACGGGA
GTCAGGCAGTAGGACGCTCTTCATTTTACTGCCTGGAGTACTTTCCTTCTCAGATGCTGCGTACCG
GAAACAACTTTACCTTCAGCTACACTTTTGAGGACGTTCCTTTCCACAGCAGCTACGCTCACAGC
CAGAGTCTGGACCGTCTCATGAATCCTCTCATCGACCAGTACCTGTATTACTTGAGCAAGACTAT
TAACGGTTCTGGACAGAATCAACAAACGCTAAAATTTTCTCAGGCCGGAGCGAGTGACATTCGG
GACCAGTCTAGGAACTGGCTTCCTGGACCCTGTTACCGCCAGCAGCGAGTATCAACCACTGTGAC
TCAAAACAACAACAGTGAATACTCGTGGACTGGAGCTACCAAGTACCACCTCAATGGCAGAGAC
TCTCTGGTGAATCCGGGCCCGGCCATGGCAAGCCACAAGGACGATGAAGAAAAGTTTTTTCCTC
AGAGCGGGGTTCTCATCTTTGGGAAGCAAGGCTCAGAGAAAACAAATGTGGACATTGAAAAGGT
CATGATTACAGACGAAGAGGAAATCAGGACAACCAATCCCGTGGCTACGGAGCAGTATGGTTCT
GTATCTACCAACCTCCAGAGCAACAGCAGAGGAGACTACAACTCCCTGAGACAAGCAGCTACCG
CAGATGTCAACACACAAGGCGTTCTTCCAGGCATGGTCTGGCAGGACAGAGATGTGTACCTTCA
GGGGCCCATCTGGGCAAAGATTCCACACACGGACGGACATTTTCACCCCTCTCCCCTCATGGGTG
GATTCGGACTTAAACACCCTCCTCCACAGATTCTCATCAAGAACACCCCGGTACCTGCGAATCCT
TCGACCACCTTCAGTGCGGCAAAGTTTGCTTCCTTCATCACACAGTACTCCACGGGACAGGTCAG
CGTGGAGATCGAGTGGGAGCTGCAGAAGGAAAACAGCAAACGCTGGAATCCCGAAATTCAGTA
CACTTCCAACTACAACAAGTCTGTTAATGTGGACTTTACTGTGGACACTAATGGCGTGTATTCAG
AGCCTCGCCCCATTGGCACCAGATACCTGACTCGTAATCTGTAA
SEQ ID NO: 33 (VP1 nucleic acid sequence of mutant 2 (5′ → 3′))
ATGGCTGCCGATGGTTATCTTCCAGATTGGCTCGAGGACACTCTCTCTGAAGGAATAAGA
CAGTGGTGGAAGCTCAAACCTGGCCCACCACCACCAAAGCCCGCAGAGCGGCATAAGGACGAC
AGCAGGGGTCTTGTGCTTCCTGGGTACAAGTACCTCGGACCCTTCAACGGACTCGACAAGGGAG
AGCCGGTCAACGAGGCAGACGCCGCGGCCCTCGAGCACGACAAAGCCTACGACCGGCAGCTCG
ACAGCGGAGACAACCCGTACCTCAAGTACAACCACGCCGACGCGGAGTTTCAGGAGCGCCTTAA
AGAAGATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTAGAGC
ACTCTCCTGTGGAGCCAGACTCCTCCTCGGGAACCGGAAAGGCGGGCCAGCAGCCTGCAAGAAA
AAGATTGAATTTTGGTCAGACTGGAGACGCAGACTCAGTACCTGACCCCCAGCCTCTCGGACAG
CCACCAGCAGCCCCCTCTGGTCTGGGAACTAATACGATGGCTACAGGCAGTGGCGCACCAATGG
CAGACAATAACGAGGGCGCCGACGGAGTGGGTAATTCCTCGGGAAATTGGCATTGCGATTCCAC
ATGGATGGGCGACAGAGTCATCACCACCAGCACCCGAACCTGGGCCCTGCCCACCTACAACAAC
CACCTCTACAAACAAATTTCCAGCCAATCAGGAGCCTCGAACGACAATCACTACTTTGGCTACAG
CACCCCTTGGGGGTATTTTGACTTCAACAGATTCCACTGCCACTTTTCACCACGTGACTGGCAAA
GACTCATCAACAACAACTGGGGATTCCGACCCAAGAGACTCAACTTCAAGCTCTTTAACATTCAA
GTCAAAGAGGTCACGCAGAATGACGGTACGACGACGATTGCCAATAACCTTACCAGCACGGTTC
AGGTGTTTACTGACTCGGAGTACCAGCTCCCGTACGTCCTCGGCTCGGCGCATCAAGGATGCCTC
CCGCCGTTCCCAGCAGACGTCTTCATGGTGCCACAGTATGGATACCTCACCCTGAACAACGGGA
GTCAGGCAGTAGGACGCTCTTCATTTTACTGCCTGGAGTACTTTCCTTCTCAGATGCTGCGTACCG
GAAACAACTTTACCTTCAGCTACACTTTTGAGGACGTTCCTTTCCACAGCAGCTACGCTCACAGC
CAGAGTCTGGACCGTCTCATGAATCCTCTCATCGACCAGTACCTGTATTACTTGAGCAGAACAAA
CACTCCAAGTGGAACCACCACGCAGTCAAGGCTTCAGTTTTCTCAGGCCGGAGCGAGTGACATT
CGGGACCAGTCTAGGAACTGGCTTCCTGGACCCTGTTACCGCCAGCAGCGAGTATCAAAGACAT
CTGCGGATAACAACAACAGTGAATACTCGTGGACTGGAGCTACCAAGTACCACCTCAATGGCAG
AGACTCTCTGGTGAATCCGGGCCCGGCCATGGCAAGCCACAAGGACGATGAAGAAAAGTTTTTT
CCTCAGAGCGGGGTTCTCATCTTTGGGAAGCAAGGCTCAGAGAAAACAAATGTGGACATTGAAA
AGGTCATGATTACAGACGAAGAGGAAATCAGGACAACCAATCCCGTGGCTACGGAGCAGTATG
GTTCTGTATCTACCAACCTCCAGAGCAACAGCAGAGGAGACTACAACTCCCTGAGACAAGCAGC
TACCGCAGATGTCAACACACAAGGCGTTCTTCCAGGCATGGTCTGGCAGGACAGAGATGTGTAC
CTTCAGGGGCCCATCTGGGCAAAGATTCCACACACGGACGGACATTTTCACCCCTCTCCCCTCAT
GGGTGGATTCGGACTTAAACACCCTCCTCCACAGATTCTCATCAAGAACACCCCGGTACCTGCGA
ATCCTTCGACCACCTTCAGTGCGGCAAAGTTTGCTTCCTTCATCACACAGTACTCCACGGGACAG
GTCAGCGTGGAGATCGAGTGGGAGCTGCAGAAGGAAAACAGCAAACGCTGGAATCCCGAAATT
CAGTACACTTCCAACTACAACAAGTCTGTTAATGTGGACTTTACTGTGGACACTAATGGCGTGTA
TTCAGAGCCTCGCCCCATTGGCACCAGATACCTGACTCGTAATCTGTAA
SEQ ID NO: 34 (VP1 nucleic acid sequence of mutant 3 (5′ → 3′))
ATGGCTGCCGATGGTTATCTTCCAGATTGGCTCGAGGACACTCTCTCTGAAGGAATAAGA
CAGTGGTGGAAGCTCAAACCTGGCCCACCACCACCAAAGCCCGCAGAGCGGCATAAGGACGAC
AGCAGGGGTCTTGTGCTTCCTGGGTACAAGTACCTCGGACCCTTCAACGGACTCGACAAGGGAG
AGCCGGTCAACGAGGCAGACGCCGCGGCCCTCGAGCACGACAAAGCCTACGACCGGCAGCTCG
ACAGCGGAGACAACCCGTACCTCAAGTACAACCACGCCGACGCGGAGTTTCAGGAGCGCCTTAA
AGAAGATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTAGAGC
ACTCTCCTGTGGAGCCAGACTCCTCCTCGGGAACCGGAAAGGCGGGCCAGCAGCCTGCAAGAAA
AAGATTGAATTTTGGTCAGACTGGAGACGCAGACTCAGTACCTGACCCCCAGCCTCTCGGACAG
CCACCAGCAGCCCCCTCTGGTCTGGGAACTAATACGATGGCTACAGGCAGTGGCGCACCAATGG
CAGACAATAACGAGGGCGCCGACGGAGTGGGTAATTCCTCGGGAAATTGGCATTGCGATTCCAC
ATGGATGGGCGACAGAGTCATCACCACCAGCACCCGAACCTGGGCCCTGCCCACCTACAACAAC
CACCTCTACAAACAAATTTCCAGCCAATCAGGAGCCTCGAACGACAATCACTACTTTGGCTACAG
CACCCCTTGGGGGTATTTTGACTTCAACAGATTCCACTGCCACTTTTCACCACGTGACTGGCAAA
GACTCATCAACAACAACTGGGGATTCCGACCCAAGAGACTCAACTTCAAGCTCTTTAACATTCAA
GTCAAAGAGGTCACGCAGAATGACGGTACGACGACGATTGCCAATAACCTTACCAGCACGGTTC
AGGTGTTTACTGACTCGGAGTACCAGCTCCCGTACGTCCTCGGCTCGGCGCATCAAGGATGCCTC
CCGCCGTTCCCAGCAGACGTCTTCATGGTGCCACAGTATGGATACCTCACCCTGAACAACGGGA
GTCAGGCAGTAGGACGCTCTTCATTTTACTGCCTGGAGTACTTTCCTTCTCAGATGCTGCGTACCG
GAAACAACTTTACCTTCAGCTACACTTTTGAGGACGTTCCTTTCCACAGCAGCTACGCTCACAGC
CAGAGTCTGGACCGTCTCATGAATCCTCTCATCGACCAGTACCTGTATTACTTGAGCAAGACTAT
TAACGGTTCTGGACAGAATCAACAAACGCTAAAATTTTCTCAGGCCGGAGCGAGTGACATTCGG
GACCAGTCTAGGAACTGGCTTCCTGGACCCTGTTACCGCCAGCAGCGAGTATCAAAGACATCTG
CGGATAACAACAACAGTGAATACTCGTGGACTGGAGCTACCAAGTACCACCTCAATGGCAGAGA
CTCTCTGGTGAATCCGGGCCCGGCCATGGCAAGCCACAAGGACGATGAAGAAAAGTTTTTTCCT
CAGAGCGGGGTTCTCATCTTTGGGAAGCAAGGCTCAGAGAAAACAAATGTGGACATTGAAAAGG
TCATGATTACAGACGAAGAGGAAATCAGGACAACCAATCCCGTGGCTACGGAGCAGTATGGTTC
TGTATCTACCAACCTCCAGAGCAACAGCAGAGGAGACTACAACTCCCTGAGACAAGCAGCTACC
GCAGATGTCAACACACAAGGCGTTCTTCCAGGCATGGTCTGGCAGGACAGAGATGTGTACCTTC
AGGGGCCCATCTGGGCAAAGATTCCACACACGGACGGACATTTTCACCCCTCTCCCCTCATGGGT
GGATTCGGACTTAAACACCCTCCTCCACAGATTCTCATCAAGAACACCCCGGTACCTGCGAATCC
TTCGACCACCTTCAGTGCGGCAAAGTTTGCTTCCTTCATCACACAGTACTCCACGGGACAGGTCA
GCGTGGAGATCGAGTGGGAGCTGCAGAAGGAAAACAGCAAACGCTGGAATCCCGAAATTCAGT
ACACTTCCAACTACAACAAGTCTGTTAATGTGGACTTTACTGTGGACACTAATGGCGTGTATTCA
GAGCCTCGCCCCATTGGCACCAGATACCTGACTCGTAATCTGTAA
SEQ ID NO: 35 (VP1 nucleic acid sequence of mutant 4 (5′ → 3′))
ATGGCTGCCGATGGTTATCTTCCAGATTGGCTCGAGGACACTCTCTCTGAAGGAATAAGA
CAGTGGTGGAAGCTCAAACCTGGCCCACCACCACCAAAGCCCGCAGAGCGGCATAAGGACGAC
AGCAGGGGTCTTGTGCTTCCTGGGTACAAGTACCTCGGACCCTTCAACGGACTCGACAAGGGAG
AGCCGGTCAACGAGGCAGACGCCGCGGCCCTCGAGCACGACAAAGCCTACGACCGGCAGCTCG
ACAGCGGAGACAACCCGTACCTCAAGTACAACCACGCCGACGCGGAGTTTCAGGAGCGCCTTAA
AGAAGATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTAGAGC
ACTCTCCTGTGGAGCCAGACTCCTCCTCGGGAACCGGAAAGGCGGGCCAGCAGCCTGCAAGAAA
AAGATTGAATTTTGGTCAGACTGGAGACGCAGACTCAGTACCTGACCCCCAGCCTCTCGGACAG
CCACCAGCAGCCCCCTCTGGTCTGGGAACTAATACGATGGCTACAGGCAGTGGCGCACCAATGG
CAGACAATAACGAGGGCGCCGACGGAGTGGGTAATTCCTCGGGAAATTGGCATTGCGATTCCAC
ATGGATGGGCGACAGAGTCATCACCACCAGCACCCGAACCTGGGCCCTGCCCACCTACAACAAC
CACCTCTACAAACAAATTTCCAGCCAATCAGGAGCCTCGAACGACAATCACTACTTTGGCTACAG
CACCCCTTGGGGGTATTTTGACTTCAACAGATTCCACTGCCACTTTTCACCACGTGACTGGCAAA
GACTCATCAACAACAACTGGGGATTCCGACCCAAGAGACTCAACTTCAAGCTCTTTAACATTCAA
GTCAAAGAGGTCACGCAGAATGACGGTACGACGACGATTGCCAATAACCTTACCAGCACGGTTC
AGGTGTTTACTGACTCGGAGTACCAGCTCCCGTACGTCCTCGGCTCGGCGCATCAAGGATGCCTC
CCGCCGTTCCCAGCAGACGTCTTCATGGTGCCACAGTATGGATACCTCACCCTGAACAACGGGA
GTCAGGCAGTAGGACGCTCTTCATTTTACTGCCTGGAGTACTTTCCTTCTCAGATGCTGCGTACCG
GAAACAACTTTACCTTCAGCTACACTTTTGAGGACGTTCCTTTCCACAGCAGCTACGCTCACAGC
CAGAGTCTGGACCGTCTCATGAATCCTCTCATCGACCAGTACCTGTATTACTTGAGCAAGACTAT
TAACGGTTCTGGACAGAATCAACAAACGCTAAAATTTTCTCAGGCCGGAGCGAGTGACATTCGG
GACCAGTCTAGGAACTGGCTTCCTGGACCCTGTTACCGCCAGCAGCGAGTATCAACCACTGTGAC
TCAAAACAACAACAGTGAATACTCGTGGACTGGAGCTACCAAGTACCACCTCAATGGCAGAGAC
TCTCTGGTGAATCCGGGCCCGGCCATGGCAAGCCACAAGGACGATGAAGAAAAGTTTTTTCCTC
AGAGCGGGGTTCTCATCTTTGGGAAGCAAGGCTCAGAGAAAACAAATGTGGACATTGAAAAGGT
CATGATTACAGACGAAGAGGAAATCAGGACAACCAATCCCGTGGCTACGGAGCAGTATGGTTCT
GTATCTACCAACCTCCAGGGACCTGGAAGAGGAGACCAGACAACACTGAGACAAGCAGCTACC
GCAGATGTCAACACACAAGGCGTTCTTCCAGGCATGGTCTGGCAGGACAGAGATGTGTACCTTC
AGGGGCCCATCTGGGCAAAGATTCCACACACGGACGGACATTTTCACCCCTCTCCCCTCATGGGT
GGATTCGGACTTAAACACCCTCCTCCACAGATTCTCATCAAGAACACCCCGGTACCTGCGAATCC
TTCGACCACCTTCAGTGCGGCAAAGTTTGCTTCCTTCATCACACAGTACTCCACGGGACAGGTCA
GCGTGGAGATCGAGTGGGAGCTGCAGAAGGAAAACAGCAAACGCTGGAATCCCGAAATTCAGT
ACACTTCCAACTACAACAAGTCTGTTAATGTGGACTTTACTGTGGACACTAATGGCGTGTATTCA
GAGCCTCGCCCCATTGGCACCAGATACCTGACTCGTAATCTGTAA
SEQ ID NO: 36 (VP1 nucleic acid sequence of mutant 5 (5′ → 3′))
ATGGCTGCCGATGGTTATCTTCCAGATTGGCTCGAGGACACTCTCTCTGAAGGAATAAGA
CAGTGGTGGAAGCTCAAACCTGGCCCACCACCACCAAAGCCCGCAGAGCGGCATAAGGACGAC
AGCAGGGGTCTTGTGCTTCCTGGGTACAAGTACCTCGGACCCTTCAACGGACTCGACAAGGGAG
AGCCGGTCAACGAGGCAGACGCCGCGGCCCTCGAGCACGACAAAGCCTACGACCGGCAGCTCG
ACAGCGGAGACAACCCGTACCTCAAGTACAACCACGCCGACGCGGAGTTTCAGGAGCGCCTTAA
AGAAGATACGTCTTTTGGGGGCAACCTCGGACGAGCAGTCTTCCAGGCGAAAAAGAGGGTTCTT
GAACCTCTGGGCCTGGTTGAGGAACCTGTTAAGACGGCTCCGGGAAAAAAGAGGCCGGTAGAGC
ACTCTCCTGTGGAGCCAGACTCCTCCTCGGGAACCGGAAAGGCGGGCCAGCAGCCTGCAAGAAA
AAGATTGAATTTTGGTCAGACTGGAGACGCAGACTCAGTACCTGACCCCCAGCCTCTCGGACAG
CCACCAGCAGCCCCCTCTGGTCTGGGAACTAATACGATGGCTACAGGCAGTGGCGCACCAATGG
CAGACAATAACGAGGGCGCCGACGGAGTGGGTAATTCCTCGGGAAATTGGCATTGCGATTCCAC
ATGGATGGGCGACAGAGTCATCACCACCAGCACCCGAACCTGGGCCCTGCCCACCTACAACAAC
CACCTCTACAAACAAATTTCCAGCCAATCAGGAGCCTCGAACGACAATCACTACTTTGGCTACAG
CACCCCTTGGGGGTATTTTGACTTCAACAGATTCCACTGCCACTTTTCACCACGTGACTGGCAAA
GACTCATCAACAACAACTGGGGATTCCGACCCAAGAGACTCAACTTCAAGCTCTTTAACATTCAA
GTCAAAGAGGTCACGCAGAATGACGGTACGACGACGATTGCCAATAACCTTACCAGCACGGTTC
AGGTGTTTACTGACTCGGAGTACCAGCTCCCGTACGTCCTCGGCTCGGCGCATCAAGGATGCCTC
CCGCCGTTCCCAGCAGACGTCTTCATGGTGCCACAGTATGGATACCTCACCCTGAACAACGGGA
GTCAGGCAGTAGGACGCTCTTCATTTTACTGCCTGGAGTACTTTCCTTCTCAGATGCTGCGTACCG
GAAACAACTTTACCTTCAGCTACACTTTTGAGGACGTTCCTTTCCACAGCAGCTACGCTCACAGC
CAGAGTCTGGACCGTCTCATGAATCCTCTCATCGACCAGTACCTGTATTACTTGAGCAAGACTAT
TAACGGTTCTGGACAGAATCAACAAACGCTAAAATTTTCTCAGGCCGGAGCGAGTGACATTCGG
GACCAGTCTAGGAACTGGCTTCCTGGACCCTGTTACCGCCAGCAGCGAGTATCAACCACTGTGAC
TCAAAACAACAACAGTGAATACTCGTGGACTGGAGCTACCAAGTACCACCTCAATGGCAGAGAC
TCTCTGGTGAATCCGGGCCCGGCCATGGCAAGCCACAAGGACGATGAAGAAAAGTTTTTTCCTC
AGAGCGGGGTTCTCATCTTTGGGAAGCAAGGCTCAGAGAAAACAAATGTGGACATTGAAAAGGT
CATGATTACAGACGAAGAGGAAATCAGGACAACCAATCCCGTGGCTACGGAGCAGTATGGTTCT
GTATCTACCAACCTCCAGGAGAATAGACGAGGCGATTTCAACAATACCAGACAAGCAGCTACCG
CAGATGTCAACACACAAGGCGTTCTTCCAGGCATGGTCTGGCAGGACAGAGATGTGTACCTTCA
GGGGCCCATCTGGGCAAAGATTCCACACACGGACGGACATTTTCACCCCTCTCCCCTCATGGGTG
GATTCGGACTTAAACACCCTCCTCCACAGATTCTCATCAAGAACACCCCGGTACCTGCGAATCCT
TCGACCACCTTCAGTGCGGCAAAGTTTGCTTCCTTCATCACACAGTACTCCACGGGACAGGTCAG
CGTGGAGATCGAGTGGGAGCTGCAGAAGGAAAACAGCAAACGCTGGAATCCCGAAATTCAGTA
CACTTCCAACTACAACAAGTCTGTTAATGTGGACTTTACTGTGGACACTAATGGCGTGTATTCAG
AGCCTCGCCCCATTGGCACCAGATACCTGACTCGTAATCTGTAA

1. A mutant of an adeno-associated virus (AAV) capsid protein, compared with a VP1 capsid protein of wild-type AAV2, comprising any one or any combination of the following mutations:
(1) in variable region IV of the VP1 capsid protein, comprising an amino acid sequence KTINGSGQNQQTLK (SEQ ID NO: 2) or an amino acid sequence having 1, 2, 3, or 4 amino acid changes when compared to SEQ ID NO: 2;
(2) in variable region V of the VP1 capsid protein, comprising an amino acid sequence TTVTQ (SEQ ID NO: 3) or an amino acid sequence having 1 or 2 amino acid changes when compared to SEQ ID NO: 3; and
(3) replacement of amino acids at positions 585-587 with a short peptide comprising an RGD sequence,
wherein an amino acid sequence of the VP1 capsid protein of the wild-type AAV2 is set forth in SEQ ID NO: 1, and
wherein the positions of the amino acids correspond to positions in the amino acid sequence of the VP1 capsid protein of the wild-type AAV2.

2. The mutant of the AAV capsid protein according to claim 1, wherein a sequence of the short peptide is SNSRGDYNSL (SEQ ID NO: 37), GPGRGDQTTL (SEQ ID NO: 38), or ENRRGDFNNT (SEQ ID NO: 39).

3. The mutant of the AAV capsid protein according to claim 1, wherein amino acids at positions 447-461 are replaced with the amino acid sequence KTINGSGQNQQTLK (SEQ ID NO: 2) or the amino acid sequence having 1, 2, 3, or 4 amino acid changes when compared to SEQ ID NO: 2, and wherein the positions of the amino acids correspond to positions in the amino acid sequence of the VP1 capsid protein of the wild-type AAV2.

4. The mutant of the AAV capsid protein according to claim 1, wherein amino acids at positions 490-494 are replaced with the amino acid sequence TTVTQ (SEQ ID NO: 3) or the amino acid sequence having 1 or 2 amino acid changes when compared to SEQ ID NO: 3, and wherein the positions of the amino acids correspond to positions in the amino acid sequence of the VP1 capsid protein of the wild-type AAV2.

5. The mutant of the AAV capsid protein according to claim 1, comprising an amino acid sequence set forth in any one of SEQ ID NOs: 27-31 and 6-8, or an amino acid sequence having at least 80%, at least 90%, at least 95%, or at least 98% sequence identity to the amino acid sequence set forth in any one of SEQ ID NOs: 27-31 and 6-8.

6. The mutant of the AAV capsid protein according to claim 1, wherein the mutant is a mutant of VP1 and/or VP2 and/or VP3 capsid protein.

7. An isolated nucleic acid molecule encoding the mutant of the AAV capsid protein according to claim 1.

8. The isolated nucleic acid molecule according to claim 7, wherein the isolated nucleic acid molecule comprises an nucleotide sequence set forth in any one of SEQ ID NOs: 32-36 and 9-12.

9. An expression vector comprising the isolated nucleic acid molecule according to claim 7.

10. A host cell comprising the isolated nucleic acid molecule according to claim 7 or the expression vector comprising the isolated nucleic acid molecule according to claim 7.

11. A host cell expressing the mutant of the AAV capsid protein according to claim 1.

12. An adeno-associated virus (AAV) comprising the mutant of the AAV capsid protein according to claim 1.

13. A method for preparing a recombinant adeno-associated virus (rAAV), comprising:
introducing at least the following components into a host cell:
(1) the isolated nucleic acid molecule according to claim 7 or an expression vector comprising the isolated nucleic acid molecule according to claims 7; and
(2) a GOI (gene of interest) plasmid comprising a target gene.

14. The method according to claim 13, wherein an expression product of the target gene is protein or RNA.

15. A rAAV prepared by the method according to claim 13.

16. The rAAV according to claim 15, wherein the rAAV has lower targeting to liver than wild-type AAV2 or wild-type AAV9; and/or
wherein the rAAV has higher targeting to muscle or heart than the wild-type AAV2 or wild-type AAV9.

17. A pharmaceutical composition comprising the rAAV according to claim 15 and a pharmaceutically acceptable carrier.

18. A method for treating a disease, comprising: administering an isolated nucleic acid molecule encoding the mutant of the AAV capsid protein according to claim 1, an expression vector, or a rAAV to a patient in need,
wherein the expression vector comprises the isolated nucleic acid molecule encoding the mutant of the AAV capsid protein according to claim 1, and
wherein the rAAV comprises the mutant of the AAV capsid protein according to claim 1.

19. The method according to claim 18, wherein the disease is a muscle-related disease or a heart-related disease.

20. The method according to claim 18, wherein the muscle-related disease is selected from the group consisting of muscular dystrophy, myasthenia gravis, polymyositis, dermatomyositis, and rhabdomyolysis; and
wherein the heart-related disease is selected from the group consisting of myocardial infarction, myocardial ischemia injury, coronary heart disease, myocardial hypertrophy, and myocardial fibrosis.


SEQUENCE LISTING (45 sequences)

SEQ ID NO: 1 - protein, 735 aa, Adeno-associated dependoparvovirus A [sequence omitted]

SEQ ID NO: 2 - protein, 14 aa, synthetic construct
KTINGSGQNQQTLK

SEQ ID NO: 3 - protein, 5 aa, synthetic construct
TTVTQ

SEQ ID NO: 4 - protein, 15 aa, synthetic construct
RTNTPSGTTTQSRLQ

SEQ ID NO: 5 - protein, 5 aa, synthetic construct
KTSAD

SEQ ID NO: 6 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTINGSGQNQQTLKFSQAGASDIRDQSRNWLPGPCYRQQRVSTTVTQNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTINGSGQNQQTLKFSQAGASDIRDQSRNWLPGPCYRQQRVSTTVTQNNNSEYSWTGATKYHLNGRDSLVNPGPAMASHKDDEEKFFPQSGVLIFGKQGSEKTNVDIEKVMITDEEEIRTTNPVATEQYGSVSTNLQAGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - protein, 731 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTINGSGQNQQTLKFSQAGASDIRDQSRNWLPGPCYRQQRVSTTVTQNNNSEYSWTGATKYHLNGRDSLVNPGPAMASHKDDEEKFFPQSGVLIFGKQGSEKTNVDIEKVMITDEEEIRTTNPVATEQYGSVSTNLQRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - DNA, 2208 bp, Adeno-associated dependoparvovirus A
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 10 - DNA, 2205 bp, synthetic construct
  source 1..2205: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcaagactattaacggttctggacagaatcaacaaacgctaaaattttctcaggccggagcgagtgacattcgggaccagtctaggaactggcttcctggaccctgttaccgccagcagcgagtatcaaccactgtgactcaaaacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 11 - DNA, 2205 bp, synthetic construct
  source 1..2205: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcaagactattaacggttctggacagaatcaacaaacgctaaaattttctcaggccggagcgagtgacattcgggaccagtctaggaactggcttcctggaccctgttaccgccagcagcgagtatcaaccactgtgactcaaaacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggctggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 12 - DNA, 2196 bp, synthetic construct
  source 1..2196: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcaagactattaacggttctggacagaatcaacaaacgctaaaattttctcaggccggagcgagtgacattcgggaccagtctaggaactggcttcctggaccctgttaccgccagcagcgagtatcaaccactgtgactcaaaacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 13 - DNA, 35 bp, synthetic construct
  source 1..35: mol_type = other DNA
catctttgaacaataaatgatttaaatcaggtatg

SEQ ID NO: 14 - DNA, 25 bp, synthetic construct
  source 1..25: mol_type = other DNA
tcaactgaaacgaatcaaccggttt

SEQ ID NO: 15 - DNA, 59 bp, synthetic construct
  source 1..59: mol_type = other DNA
tgttgattctgtccagaaccgttaatagtcttgctcaagtaatacaggtactggtcgat

SEQ ID NO: 16 - DNA, 53 bp, synthetic construct
  source 1..53: mol_type = other DNA
acggttctggacagaatcaacaaacgctaaaattttctcaggccggagcgagt

SEQ ID NO: 17 - DNA, 46 bp, synthetic construct
  source 1..46: mol_type = other DNA
gttgttttgagtcacagtggttgatactcgctgctggcggtaacag

SEQ ID NO: 18 - DNA, 50 bp, synthetic construct
  source 1..50: mol_type = other DNA
atcaaccactgtgactcaaaacaacaacagtgaatactcgtggactggag

SEQ ID NO: 19 - DNA, 44 bp, synthetic construct
  source 1..44: mol_type = other DNA
ttgtctgttgccagcctggaggttggtagatacagaaccatact

SEQ ID NO: 20 - DNA, 37 bp, synthetic construct
  source 1..37: mol_type = other DNA
cctccaggctggcaacagacaagcagctaccgcagat

SEQ ID NO: 21 - DNA, 39 bp, synthetic construct
  source 1..39: mol_type = other DNA
ctgcttgtctctggaggttggtagatacagaaccatact

SEQ ID NO: 22 - DNA, 31 bp, synthetic construct
  source 1..31: mol_type = other DNA
caacctccagagacaagcagctaccgcagat

SEQ ID NO: 23 - DNA, 20 bp, synthetic construct
  source 1..20: mol_type = other DNA
aaccagcgccattctgatca

SEQ ID NO: 24 - DNA, 20 bp, synthetic construct
  source 1..20: mol_type = other DNA
tcggggttgttaacgtagcc

SEQ ID NO: 25 - DNA, 21 bp, synthetic construct
  source 1..21: mol_type = other DNA
caggagagtgtttcctcgtcc

SEQ ID NO: 26 - DNA, 20 bp, synthetic construct
  source 1..20: mol_type = other DNA
ttcccattctcggccttgac

SEQ ID NO: 27 - protein, 741 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTINGSGQNQQTLKFSQAGASDIRDQSRNWLPGPCYRQQRVSTTVTQNNNSEYSWTGATKYHLNGRDSLVNPGPAMASHKDDEEKFFPQSGVLIFGKQGSEKTNVDIEKVMITDEEEIRTTNPVATEQYGSVSTNLQSNSRGDYNSLRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 28 - protein, 742 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQSNSRGDYNSLRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 29 - protein, 741 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTINGSGQNQQTLKFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQSNSRGDYNSLRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 30 - protein, 741 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTINGSGQNQQTLKFSQAGASDIRDQSRNWLPGPCYRQQRVSTTVTQNNNSEYSWTGATKYHLNGRDSLVNPGPAMASHKDDEEKFFPQSGVLIFGKQGSEKTNVDIEKVMITDEEEIRTTNPVATEQYGSVSTNLQGPGRGDQTTLRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 31 - protein, 741 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTINGSGQNQQTLKFSQAGASDIRDQSRNWLPGPCYRQQRVSTTVTQNNNSEYSWTGATKYHLNGRDSLVNPGPAMASHKDDEEKFFPQSGVLIFGKQGSEKTNVDIEKVMITDEEEIRTTNPVATEQYGSVSTNLQENRRGDFNNTRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 32 - DNA, 2226 bp, synthetic construct
  source 1..2226: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcaagactattaacggttctggacagaatcaacaaacgctaaaattttctcaggccggagcgagtgacattcgggaccagtctaggaactggcttcctggaccctgttaccgccagcagcgagtatcaaccactgtgactcaaaacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagcaacagcagaggagactacaactccctgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 33 - DNA, 2229 bp, synthetic construct
  source 1..2229: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagcaacagcagaggagactacaactccctgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 34 - DNA, 2226 bp, synthetic construct
  source 1..2226: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcaagactattaacggttctggacagaatcaacaaacgctaaaattttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagcaacagcagaggagactacaactccctgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 35 - DNA, 2226 bp, synthetic construct
  source 1..2226: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcaagactattaacggttctggacagaatcaacaaacgctaaaattttctcaggccggagcgagtgacattcgggaccagtctaggaactggcttcctggaccctgttaccgccagcagcgagtatcaaccactgtgactcaaaacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagggacctggaagaggagaccagacaacactgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 36 - DNA, 2226 bp, synthetic construct
  source 1..2226: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcaagactattaacggttctggacagaatcaacaaacgctaaaattttctcaggccggagcgagtgacattcgggaccagtctaggaactggcttcctggaccctgttaccgccagcagcgagtatcaaccactgtgactcaaaacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggagaatagacgaggcgatttcaacaataccagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 37 - protein, 10 aa, synthetic construct
SNSRGDYNSL

SEQ ID NO: 38 - protein, 10 aa, synthetic construct
GPGRGDQTTL

SEQ ID NO: 39 - protein, 10 aa, synthetic construct
ENRRGDFNNT

SEQ ID NO: 40 - DNA, 55 bp, synthetic construct
  source 1..55: mol_type = other DNA
gagttgtagtctcctctgctgttgctctggaggttggtagatacagaaccatact

SEQ ID NO: 41 - DNA, 50 bp, synthetic construct
  source 1..50: mol_type = other DNA
gcaacagcagaggagactacaactccctgagacaagcagctaccgcagat

SEQ ID NO: 42 - DNA, 56 bp, synthetic construct
  source 1..56: mol_type = other DNA
tgttgtctggtctcctcttccaggtccctggaggttggtagatacagaaccatact

SEQ ID NO: 43 - DNA, 48 bp, synthetic construct
  source 1..48: mol_type = other DNA
cctggaagaggagaccagacaacactgagacaagcagctaccgcagat

SEQ ID NO: 44 - DNA, 56 bp, synthetic construct
  source 1..56: mol_type = other DNA
attgttgaaatcgcctcgtctattctcctggaggttggtagatacagaaccatact

SEQ ID NO: 45 - DNA, 48 bp, synthetic construct
  source 1..48: mol_type = other DNA
aatagacgaggcgatttcaacaataccagacaagcagctaccgcagat